SEQUENCE LISTING (36 sequences)

SEQ ID NO: 1 - protein, 15 aa, Artificial Sequence
  Synthetic linker
GGGGSGGGGSGGGGS

SEQ ID NO: 2 - protein, 10 aa, Artificial Sequence
  Synthetic linker
GGGGSGGGGS

SEQ ID NO: 3 - protein, 15 aa, Artificial Sequence
  Synthetic linker
ESGRSGGGGSGGGGS

SEQ ID NO: 4 - protein, 14 aa, Artificial Sequence
  Synthetic linker
EGKSSGSGSESKST

SEQ ID NO: 5 - protein, 15 aa, Artificial Sequence
  Synthetic linker
EGKSSGSGSESKSTQ

SEQ ID NO: 6 - protein, 14 aa, Artificial Sequence
  Synthetic linker
EGKSSGSGSESKVD

SEQ ID NO: 7 - protein, 14 aa, Artificial Sequence
  Synthetic linker
GSTSGSGKSSEGKG

SEQ ID NO: 8 - protein, 18 aa, Artificial Sequence
  Synthetic linker
KESGSVSSEQLAQFRSLD

SEQ ID NO: 9 - protein, 16 aa, Artificial Sequence
  Synthetic linker
ESGSVSSEELAFRSLD

SEQ ID NO: 10 - protein, 17 aa, Artificial Sequence
  Synthetic peptide
MWTLVSWVALTAGLVAG

SEQ ID NO: 11 - protein, 25 aa, Artificial Sequence
  Synthetic peptide
CPDGQFCPVACCLDPGGASYSCCRP

SEQ ID NO: 12 - protein, 52 aa, Artificial Sequence
  Synthetic peptide
CQVDAHCSAGHSCIFTVSGTSSCCPFPEAVACGDGHHCCPRGFHCSADGRSC

SEQ ID NO: 13 - protein, 53 aa, Artificial Sequence
  Synthetic peptide
CPDSQFECPDFSTCCVMVDGSWGCCPMPQASCCEDRVHCCPHGAFCDLVHTRC

SEQ ID NO: 14 - protein, 53 aa, Artificial Sequence
  Synthetic peptide
CPDARSRCPDGSTCCELPSGKYGCCPMPNATCCSDHLHCCPQDTVCDLIQSKC

SEQ ID NO: 15 - protein, 52 aa, Artificial Sequence
  Synthetic peptide
CDMEVSCPDGYTCCRLQSGAWGCCPFTQAVCCEDHIHCCPAGFTCDTQKGTC

SEQ ID NO: 16 - protein, 51 aa, Artificial Sequence
  Synthetic peptide
CDNVSSCPSSDTCCQLTSGEWGCCPIPEAVCCSDHQHCCPQGYTCVAEGQC

SEQ ID NO: 17 - protein, 52 aa, Artificial Sequence
  Synthetic peptide
CDQHTSCPVGQTCCPSLGGSWACCQLPHAVCCEDRQHCCPAGYTCNVKARSC

SEQ ID NO: 18 - protein, 52 aa, Artificial Sequence
  Synthetic peptide
CGEGHFCHDNQTCCRDNRQGWACCPYRQGVCCADRRHCCPAGFRCAARGTKC

SEQ ID NO: 19 - protein, 21 aa, Artificial Sequence
  Synthetic peptide
LRREAPRWDAPLRDPALRQLL

SEQ ID NO: 20 - DNA, 7018 bp, Homo sapiens
ggcgggcgcgccgggcggcaggtgtcggcgtcggcggcattcggcggcgatggagcggccctggggagctgcggacggcctctcgcgctggccccatggcctcggcctcctcctcctcctgcagctgctgccgccgtcgaccctcagccaggaccggctggacgcgccgccgccgcccgctgcgccgctgccgcgctggtctggccccatcggggtgagctgggggctgcgggcggccgcagccgggggcgcgtttccccgcggcggccgttggcgtcgcagcgcgccgggcgaggacgaggagtgcggccgggtccgggacttcgtcgccaagctggccaacaacacgcaccagcatgtgtttgatgatctcagaggctcagtatccttgtcctgggttggagatagcactggggtcattctagtcttgactaccttccatgtaccactggtaattatgacttttggacagtccaagctatatcgaagtgaggattatgggaagaactttaaggatattacagatctcatcaataacacctttattcggactgaatttggcatggctattggtcctgagaactctggaaaggtggtgttaacagcagaggtgtctggaggaagtcgtggaggaagaatcttcagatcatcagattttgcgaagaattttgtgcaaacagatctcccttttcatcctctcactcagatgatgtatagccctcagaattctgattatcttttagctctcagcactgaaaatggcctgtgggtgtccaagaattttgggggaaaatgggaagaaatccacaaagcagtatgtttggccaaatggggatcagacaacaccatcttctttacaacctatgcaaatggctcctgcaaagctgaccttggggctctggaattatggagaacttcagacttgggaaaaagcttcaaaactattggtgtgaaaatctactcatttggtcttgggggacgtttcctttttgcctctgtgatggctgataaggataccacaagaaggatccacgtttcaacagatcaaggggacacatggagcatggcccagctcccctccgtgggacaggaacagttctattctattctggcagcaaatgatgacatggtattcatgcatgtagatgaacctggagacactgggtttggcacaatctttacctcagatgatcgaggcattgtctattccaagtctttggaccgacatctctacactaccacaggcggagagacggactttaccaacgtgacctccctccgcggcgtctacataacaagcgtgctctccgaagataattctatccagaccatgatcacttttgaccaaggaggaaggtggacgcacctgaggaagcctgaaaacagtgaatgtgatgctacagcaaaaaacaagaatgagtgcagccttcatattcatgcttcctacagcatctcccagaaactgaatgttccaatggccccactctcagagccgaatgccgtaggcattgtcattgctcatggtagcgtgggggatgccatctcagtgatggttccagatgtgtacatctcagatgatgggggttactcctggacaaagatgctggaaggaccccactattacaccatcctggattctggaggcatcattgtggccattgagcacagcagccgtcctatcaatgtgattaagttctccacagacgaaggtcaatgctggcaaacctacacgttcaccagggaccccatctatttcactggcctagcttcagaacctggagctaggtccatgaatatcagcatttggggcttcacagaatctttcctgaccagccagtgggtctcctacaccattgattttaaagatatccttgaaaggaactgtgaagagaaggactataccatatggctggcacactccacagaccctgaagattatgaagatggctgcattttgggctacaaagaacagtttctgcggctacgcaagtcatccgtgtgtcagaatggtcgagactatgttgtgaccaagcagccctccatctgcctctgttccctggaggactttctctgtgattttggctactaccgtccagaaaatgactccaagtgtgtggaacagccagaactgaagggccacgacctggagttttgtctgtacggaagagaagaacacctaacaacaaatgggtaccggaaaattccaggggacaaatgccagggtggggtaaatccagttcgagaagtaaaagacttgaaaaagaaatgcacaagcaactttttgagtccggaaaaacagaattccaagtcaaattctgttccaattatcctggccatcgtgggattgatgctggtcacagtcgtagcaggagtgctcattgtgaagaaatatgtctgtgggggaaggttcctggtgcatcgatactctgtgctgcagcagcatgcagaggccaatggtgtggatggtgtggatgctttggacacagcctcccacactaataaaagtggttatcatgatgactcagatgaggacctcttggaatagctcttcagaggagctggacccagcatggatggtggaaccacagtacctcttacactccctgtggctccaacttcaggaaataaatttcccattgcgagggacccagctctgtttctgctgcttccatcaaagccaaaaggacctacactaaagaaatgcagggtgggggtggggaaccctgagcacttttttacaattggctctgagaaaaagggagacattttaaattctttaacttcttatttctcgtcctgtctctttgcaaagtatgggcttttttgtttttgttttttaagggaaacgaaatggaattcgaagggaccttttcactaaccccacttctgtgtgttctgcatggcgcctgccccagggcatctgccaactccagtatcagctctcacagtgtacttggtaccatccctgggctctgctggcgagacgaaacagctgtagagatgaaaacaggctgcagaggctggcacagcctggccggcttttctccatctggggacagtcctactccaagaacactgcacaccagctcctcacacagatcccacttactctttttttttttttcagagaccacagaccacagtgatttttcttttcccttgtttaattaggcaatacccttgttaattgccctttggcaactaacttaaccatgtgcttcccacacagtacatcaggaaaacttacagggcaatatttttaacttggggcaggaagaagggagcagcagagaattgactagatatagcacctattaaaagagaactcttgcttcttctgagatttttcaagctgtgctttgtgtgtgtgccagtagacttacgcaaggacagggtacaaacttagctggaagtctgcccaggctgaatgatctcttccctagagttgattgtcgggtacacagtgtgaacccccgaagacggaacctcacagtcttccatgttcccttcttaactgtcgtgtggctcgttgctaaatcatgacaatggctgcctatctgctgcttcttaggttgctgttgtacatggaaccaggactagagattttttcagatttatagacttaaaaaattagaattttattaccaggctttccttctcaccccttttttctgactttgccaagtaatttgttgacacgaaaattttggaggaaccaattgaaaacacacttccagtctagatgatgctttgtgtgatacattaagttcttattttggattaaaagaagttttccatttgatacttctctaaattaaataaattatagaatgtagttgggtggattttggggtggccatatagtaatggaaagctgcaataattagttttaatacagcttgaatatttgctatatagaaatatagtatggaaagtttttggtcttaatgtagctactgtgcgggtcacagtttctcccaatgattatgactgggacattctttggtagataccatttgctactagtttattttgtggctagaaagtcagttttgtgtgtttttttttttttttatttgaagtgccaaattaactttagtcagaatgtgagcagatggctaagttctctcctccccagaatggattaacagctgcgtggaaagtgggggagagagtggatggagacttttagagatgttaaaactgcagtagaatgaaatgagtcagggagcttcagttagaaaataaagttgaggcagtttttgtgaagataatatggttagggctggagtgcactagtctttttgcttattcattttgcatggttttaaaattaaaaataattccgaagatacaccagctcacaaatgaaaacgtcagcctctgccccaccctccctcctgcccaaagtgaatttggtactcagaaaagaactgtttataccactcacctttctcccagcatgtactcactgtgggcagatgcaccaatacatggtaatcctcttactcattttaagacgtaggaaactcaatattcttctctaaccatatacgatagggctcttcgcttttaatgatatctgggatttctgtggaacttggcaaattttcagagcaccttcactcacataatgtcatttgaacctcacaatgttcttgggatggagtcagttgttcagggtccccgtgtgtgtgataagcagtgctggctggctgtcttcagaactcttggaaatctttacacatgcgagtgctaaccactttgagcaaggctgccttcttgtagatgacttgctgttctttatgacagggatcagtggcatttgtttcctagcagtatttagcacctttttgccaccttggtgaacagaaaattgtattttcctgtctttcatggctgaaaacaaaagtaatgggaattttaaatacgtttgcagaaactgcccctcccctcattgagggtcactgctcaagagtgcaggagtggactctccactgatgggtctccctccccatcctggtttccaccccgggctggctagctctgttggtttgaagactgacagccagcctggctcattctcattattggctagttagctttctttatcaacctgctcactcacaaatgtgtgccctcagccagagagtaagaaagcccaaatctgttacagcttctaaaaaaatagatttctaatttgtcctactcatgttaggagcattatctttgaaggtaaaacatagtgtatcattgtgtaaactcccaggcttgatgtagcagaagagatcatttctggaggcttcagcaatggaatttagcattataagagagattggacaaaccagtccaaagtggtccgagttcttaaatccaggtagggaactcactcttctttcttctctggacctaattgggcattgggctttagtgagaccacagaccaggcccgtctctcctgtaggcttttaattcaatggcaactctatttcaaagaataaaagcctttggagagttgcggcagttctgggggcgggctcaggagagtccatagatcagccgtaactggaacgtagaatctacgtctgcctctgaatggacttcccacctcctctctcttgctctgatgcttgcctctgggcctctccatgcccaaggtggtctttcatccttgacaggctggtaatgtgctggccacctccagctcctgcatcgagtctgtaaaccagagctggttctcatggccttcgtcacgataccaggatacggaggggagcccagggccatccatacccaccccagggtaacggggctggcctggcattagtcattatttagtttccaggccaaccatccagatagagattccctctttcctttgagcagtgctctcaagagctccgtgcctgtccacaatgacctagagtgcatcctgctcattgtcagtgtagcccctcgcccctatattcatccaggatacttggaagtgctaaaataggaagggattcggctttcaactttgctaccatcttccctgaagcaggaaaatgaacatggacttaaatgttctttgaaaaaaccaaagttttaagatttgctgtgtgatgaagtgacagggagggccggagtcagcaggtgccagactttctgttctgtctgccatgggtttgtccagctcaggtagctctaggagcaccatcctgccctagcagagcccaggccttgccctcatgaagcatcattgaaatagcaggagcatgttgatttcttggttaggttgcattataataacaagagtcagaacattaattcgaaacaacttgcagtatgcatttcttcacaccagtacattcttaagtgtacttgtttataaggaataacataaactaatctgtacctttatatatatgtgtgtgtacatatatacatatataaactgtatagtgtacatggtaatgatttattgctatgccccagatccttaatgtagttctcatcctccgcatgccctcagccacaagcgggtgactgactgttccctgatgatttggcccacctcctgtgtttggacctctagggaggagggttttggtcatactctccttatcctcgtgcacagaaatgctcagggtccccatgtgcctgttgttcagccctctctcttgttccctttctgagcatgtggtccttccccaggctgtgggacagctgccttcccacgaaagtgtaaagcagtattaagatcattactgcatgtgccctaaaaacccaagttttctattcccttaggacagaaaattgcatgtgaggtgggataatcgagtttcagtgacccacgtcagttacacattaaagccagaccccatgataaaattccacaaaatggaaataaaactcaaatttctttagcattgtgtaaataaatctgaatgtgtttaactttgtactggtaattttctgtatatttggaatatttgggttaaaaataaaacagactggactttgttacctgacctac

SEQ ID NO: 21 - protein, 831 aa, Homo sapiens
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

SEQ ID NO: 22 - protein, 8 aa, Artificial Sequence
  Synthetic FLAG peptides
DYKDDDDK

SEQ ID NO: 23 - protein, 8 aa, Artificial Sequence
  Synthetic FLAG peptides
DYKDEDDK

SEQ ID NO: 24 - protein, 9 aa, Artificial Sequence
  Synthetic Strep epitope
AWRHPQFGG

SEQ ID NO: 25 - protein, 11 aa, Artificial Sequence
  Synthetic VSV-G epitope
YTDIEMNRLGK

SEQ ID NO: 26 - protein, 6 aa, Artificial Sequence
  Synthetic poly-His epitope
HHHHHH

SEQ ID NO: 27 - protein, 13 aa, Influenza virus
YPYDVPDYAIEGR

SEQ ID NO: 28 - protein, 11 aa, Homo sapiens
EQKLLSEEDLN

SEQ ID NO: 29 - DNA, 2323 bp, Homo sapiens
ggcgagaggaagcagggaggagagtgatttgagtagaaaagaaacacagcattccaggctggccccacctctatattgataagtagccaatgggagcgggtagccctgatccctggccaatggaaactgaggtaggcgggtcatcgcgctggggtctgtagtctgagcgctacccggttgctgctgcccaaggaccgcggagtcggacgcaggcagaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgagggacagtactgaagactctgcagccctcgggaccccactcggagggtgccctctgctcaggcctccctagcacctccccctaaccaaattctccctggaccccattctgagctccccatcaccatgggaggtggggcctcaatctaaggccttccctgtcagaagggggttgtggcaaaagccacattacaagctgccatcccctccccgtttcagtggaccctgtggccaggtgcttttccctatccacaggggtgtttgtgtgtgtgcgcgtgtgcgtttcaataaagtttgtacactttcaaaaaaaaaaaaaaaaaa

SEQ ID NO: 30 - protein, 593 aa, Homo sapiens
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 31 - DNA, 2348 bp, Mus musculus
ggggagaaagaggaaagggaggaggaatgtgaactgagtagaaaagaaacacagcattccaggagagtcccgcctctacgtagacaagtaaccaatggaagccctggatctccgagcaatggctaatggaaattgaggtgggcgggccatcgtgcggccaagccctagtcctggagctgaccgccagatgcctcccagggagcccggaccccgacgcaggcagaccatgtgggtcctgatgagctggctggccttcgcggcagggctggtagccggaacacagtgtccagatgggcagttctgccctgttgcctgctgccttgaccagggaggagccaactacagctgctgtaaccctcttctggacacatggcctagaataacgagccatcatctagatggctcctgccagacccatggccactgtcctgctggctattcttgtcttctcactgtgtctgggacttccagctgctgcccgttctctaagggtgtgtcttgtggtgatggctaccactgctgcccccagggcttccactgtagtgcagatgggaaatcctgcttccagatgtcagataaccccttgggtgctgtccagtgtcctgggagccagtttgaatgtcctgactctgccacctgctgcattatggttgatggttcgtggggatgttgtcccatgccccaggcctcttgctgtgaagacagagtgcattgctgtccccatggggcctcctgtgacctggttcacacacgatgcgtttcacccacgggcacccacaccctactaaagaagttccctgcacaaaagaccaacagggcagtgtctttgcctttttctgtcgtgtgccctgatgctaagacccagtgtcccgatgattctacctgctgtgagctacccactgggaagtatggctgctgtccaatgcccaatgccatctgctgttccgaccacctgcactgctgcccccaggacactgtatgtgacctgatccagagtaagtgcctatccaagaactacaccacggatctcctgaccaagctgcctggatacccagtgaaggaggtgaagtgcgacatggaggtgagctgccctgaaggatatacctgctgccgcctcaacactggggcctggggctgctgtccatttgccaaggccgtgtgttgtgaggatcacattcattgctgcccggcagggtttcagtgtcacacagagaaaggaacctgcgaaatgggtatcctccaagtaccctggatgaagaaggtcatagcccccctccgcctgccagacccacagatcttgaagagtgatacaccttgtgatgacttcactaggtgtcctacaaacaatacctgctgcaaactcaattctggggactggggctgctgtcccatcccagaggctgtctgctgctcagacaaccagcattgctgccctcagggcttcacatgtctggctcaggggtactgtcagaagggagacacaatggtggctggcctggagaagatacctgcccgccagacaaccccgctccaaattggagatatcggttgtgaccagcataccagctgcccagtagggcaaacctgctgcccaagcctcaagggaagttgggcctgctgccagctgccccatgctgtgtgctgtgaggaccggcagcactgttgcccggccgggtacacctgcaatgtgaaggcgaggacctgtgagaaggatgtcgattttatccagcctcccgtgctcctgaccctcggccctaaggttgggaatgtggagtgtggagaagggcatttctgccatgataaccagacctgttgtaaagacagtgcaggagtctgggcctgctgtccctacctaaagggtgtctgctgtagagatggacgtcactgttgccccggtggcttccactgttcagccaggggaaccaagtgtttgcgaaagaagattcctcgctgggacatgtttttgagggatccggtcccaagaccgctactgtaaggaagggctacagacttaaggaactccacagtcctgggaaccctgttccgagggtacccactactcaggcctccctagcgcctcctcccctaacgtctccccggcctactcatcctgagtcaccctatcaccatgggaggtggagcctcaaactaaaaccttcttttatggaaagaaggctgtggccaaaagccccgtatcaaactgccatttcttccggtttctgtggaccttgtggccaggtgctcttcccgagccacaggtgttctgtgagcttgcttgtgtgtgtgtgcgcgtgtgcgtgtgttgctccaataaagtttgtacactttctgaaaaaaaaaaaaaaaaa

SEQ ID NO: 32 - protein, 602 aa, Mus musculus
MPPREPGPRRRQTMWVLMSWLAFAAGLVAGTQCPDGQFCPVACCLDQGGANYSCCNPLLDTWPRITSHHLDGSCQTHGHCPAGYSCLLTVSGTSSCCPFSKGVSCGDGYHCCPQGFHCSADGKSCFQMSDNPLGAVQCPGSQFECPDSATCCIMVDGSWGCCPMPQASCCEDRVHCCPHGASCDLVHTRCVSPTGTHTLLKKFPAQKTNRAVSLPFSVVCPDAKTQCPDDSTCCELPTGKYGCCPMPNAICCSDHLHCCPQDTVCDLIQSKCLSKNYTTDLLTKLPGYPVKEVKCDMEVSCPEGYTCCRLNTGAWGCCPFAKAVCCEDHIHCCPAGFQCHTEKGTCEMGILQVPWMKKVIAPLRLPDPQILKSDTPCDDFTRCPTNNTCCKLNSGDWGCCPIPEAVCCSDNQHCCPQGFTCLAQGYCQKGDTMVAGLEKIPARQTTPLQIGDIGCDQHTSCPVGQTCCPSLKGSWACCQLPHAVCCEDRQHCCPAGYTCNVKARTCEKDVDFIQPPVLLTLGPKVGNVECGEGHFCHDNQTCCKDSAGVWACCPYLKGVCCRDGRHCCPGGFHCSARGTKCLRKKIPRWDMFLRDPVPRPLL

SEQ ID NO: 33 - DNA, 6804 bp, Mus musculus
tcggcagcgatggagcggccccggggagctgcggacggccttttgcgctggcccctcggcctcctcctgctccttcaactgctgcctcctgccgccgtcggccaggaccggctggacgcgccgccgccgcccgcgcctcctctgctgcgctgggccggtccggtcggggtgagctgggggctgcgcgccgccgcgcccgggggccccgtcccccgcgctggccgttggcgccgcggcgcgcccgccgaggaccaagactgcggccgcctcccggacttcatcgccaagctgaccaacaatacgcaccagcatgtctttgatgacctcagtggctcagtgtccttgtcctgggttggagacagcactggggttattctcgtcctgaccactttccaagtgcctctggtaattgtgagctttggacagtccaagttgtatcgaagtgaggattatggaaagaactttaaggatattacaaatctcatcaataacaccttcattcggacggaatttggcatggctattggtcctgagaactctggaaaggtgatactaacagcggaggtgtccgggggaagccgaggcggaagagtgttcaggtcatcagactttgccaagaactttgtgcaaacagatctcccctttcatcctctgacgcagatgatgtacagccctcagaattctgattacctgttagctctcagcaccgaaaatggcctgtgggtgtccaagaattttggggaaaaatgggaagaaatccacaaagcagtatgtttggccaaatggggaccaaacaacatcatcttctttaccacccatgtgaatggctcctgcaaagctgatcttggtgccctggaattatggagaacatccgacttgggaaaaaccttcaaaaccattggtgtgaaaatctactcctttggtcttgggggccgtttcctttttgcctctgtgatggctgataaggacacaacaagaaggatccatgtgtcaacagaccagggggacacatggagcatggcacaacttccttctgtgggacaggaacagttctactccatcctggcagccaatgaggacatggtcttcatgcatgtagatgaacctggagataccgggtttggcaccatctttacctctgatgatcgaggcattgtctactccaagtctctggacagacatctctataccaccacaggcggggagacggactttaccaacgtgacttccctccgtggggtctatataacaagcacgctctcagaagataactctattcagagcatgatcacttttgaccagggaggacggtgggagcacctgcggaagccggagaacagcaagtgcgacgctaccgcaaagaacaagaacgagtgcagccttcatatccatgcttcttatagcatctcccagaagctaaacgttccaatggccccactttccgagcccaatgctgtgggcatagtcatcgctcacggtagtgtgggagatgccatctcggtgatggtcccagatgtgtacatctcagatgatgggggttactcctgggcgaagatgctagaaggaccacattactataccatcctggactctggaggcatcattgtggccattgagcacagcaaccgtcctatcaatgtgattaagttctccacagatgaaggccagtgctggcagagctatgtgttcacacaggagcccatctacttcactgggcttgcttccgagcctggagccaggtccatgaacatcagcatctggggattcacagagtctttcattacccgccagtgggtctcctacacagtcgatttcaaagacatccttgagcggaattgtgaagaggatgactataccacgtggctggcacactccacagaccctggagattacaaagacggctgcattttgggctataaagaacagttcctacggctacggaagtcatccgtctgtcagaatggtcgagactatgttgtggccaagcagccatccgtctgtccgtgttccctggaggacttcctctgtgactttggctacttccgtccggagaacgcctcagagtgcgtggagcagcctgaactgaaggggcatgagttagagttctgtctgtacggcaaggaggagcacctgacaacaaatgggtaccggaaaatcccaggagacaaatgccaaggtgggatgaatcccgccagagaagtaaaagacttgaaaaagaaatgcacaagcaacttcttgaaccccacaaagcagaattccaagtcaaattctgtccctattatcctggccatcgtgggactgatgcttgtcacagtcgtagcaggagtcctcattgtgaagaaatatgtctgtggcggaaggttcctggtgcaccggtactcggtgctacagcagcacgcagaggctgacggcgtagaggctttggattcaacctcccacgctaaaagcggatatcacgacgactcagatgaggacctcctggaatagctcttcagaggagctgggactgcgtgtgcgtggagccacagtacctcctacactccctgaggctccgacttggggaaataaatttcccacctcgtcggccccagctctgtttctgccactcctgtccaagcccaaaggacctacactaaagaaagcaggatgggggtgggggaccccgactgacctcacagttggctctgaggagaggggggaaattttaaattctttaacatttttgtttcaatttctgtcttttgtaagactgtgggctttaggcttctcttggtttgttttgtgcattaagggacataacttgggactcaaagggattgtttcgctgaccccgctcttgtgtgttccctctggtgcccaccccagggcatctgccaactccagcatcaactcaactcccaccgcatacctggtgctgtccctgggctctgctggtcatgtgaggcagcctcggagaaggaagcagagggtgtaatgactggcacacctggacagcttttctccatccgcagacagccctgttcagagagtaccgagcaccagccctgtgcggcttcctcatccccacctcacccccaaccactactgtagggcacaatgacttttcttttcccgtttaattaggcaatacccttgttaattgcccgttggcaactaacttaatcatgtgcttccaacacactaaatagggaaaacttacagggcaatatttttaacttgtggcaagagggaggcagagaattgactagctctgacacctattaaagacaaaactggctgcttctgagctctttcaggctgtactttgtgctgtgccagtggctctgcccaggctccgccatccctttactggagttcattggtcagtgcacagtgcagccacaggactgaccgtcgtaggcttccatttttcccatcacccactgtaaaggctcagagttgaactaccactgtggctgcttattgcttcctcctaagttgctactgttcatagaactgggactgggaatgttttcaggttgatggactttttcaagctcaaagttatgttcccagactatcctcacccttcccctagttttgctgagttatttgttgtccagaacctgttggctaaccccactgaaggcacatttctagatgatgccgtgtgtggtcaatacattgctttcatgttttaaatttaaggaaaaattttatctgatatttttccaaattaaataagttagagaatgcaatcggcggatgtgggggcggccatctggtagtggaaagctgtagcgatctgagtacagcttagatgttagctggagaggaagatggtgtggcaggtctctggtctccctacccctgctgtgctgatcacagtccctcccagttaccactaggacattctctagtagatccattttgctgtcactttattctatggctagaaagtcagtggaaagtaaatgtgccaaactcacttgtcagaacacacgagcagatggcagagtcctcctccccagcaggaacaaacggcatcagggaaagagggggaggaagaggagggagaagaagaggagggagagccaaagagatgtcaactgcagctcagtgagtcatggaacgtcagttagaaaacgaagttagggtcgtttctgggaagagaatcaatacctgttagagccagaatgctccaagtcttttagcttgttcattttgtaatgattctaaaataattccaaagatatagcagctcataaatttatcaaaagttctgtccaccctcacctcctgccaaggggagagatctatttctactacacgctgtctctcccagtacatccccggattggtgtgggtagataggccaacgtatggtgatctcccctagtgtctggatggacttaatgctcatccctgaccagagactagggtgctctgcctctaatggtttctgggatttctgtagagcttggcagtttctggctcatttctcttgagcaccgcactattcttgccatagagagagcccaggagccaacgctgaatcaggtgcccggagtctttggagctctgaaactctcggctatgcaagaccccgggtccttgcagcagagcttctcatgggtggcttgctggggccgggccgaggactcctcagaggcgtttcctgcagagccattattttgacgcctttttgttttctttttgaaaaacaaaatattattcccctgcttcatggctgaaataaaaggtgatggtaactgggggcaccccctctccctgggatagccactgtcggggagcacaggaccagcctctggatcagtccctctctgccccttcacttctgtctctagctcactggctctgtgggcttgggctctgcccctacaacggctctcagaagcattcacacgcacagatgtgcaacctcagccagaagttgaagctctttaagccattttatttattttgttttttacagaatttgtgctttggtttgccctgcgcagccttggagcatcataagcctggctggtgggcatagttattctcagcattgtggtggcaaatgacgttggttctagagacttagagacttgggcaataggatttaacactgctaggtagctcagacatatcagaaaatccatagtggcctgagttgtaaatccaaaacatatttttgttggcttgggacgttaggggcttggggttttcctgaggcaagaggtcatggctgaatgggcctgtgccgtgaattgaatggaacactactttgaggcccttggagagtgacagcagctatagggtggactcaggagctgtcagtcttaacagctgagccttggaccccactgcctctgagtctacattctggcaggcagtcccatgctctgggccttacctctagtcccctgcctgctcacacctggtcccctagccttttctcttgggcagtgtagtagtatgggcgagccaccctcagctcttttattggccggggctataaaccatggctgggcataactattccgaactgtccccccatcaggatacaagatgctgagagggctgcagggccattccatcaagccccagggtgaaagggctggcatggcatcgctcattttattgctctgcaggtaaactgtataggtatagagtcctccagtgctctgaagagccctgcctctatgcacgcccccggcaaagggcttgtcagtaggtccccctcaactccatactcaacagggatgcacaggattgctaacagagggcggggcacacctcagttttctatcaccttcctaccagaggggaaagtaacctgaactttaatgtcttttggaacaaaaccaaagtttaagatttgccatatgacgacgtggtgaccgggaagtccagagtcggcaagcaggtgccagattttctatcccatcagccagtgcttcaccaggtgcaggctgctatgggcttgtggaaaccacaggtacaggtgggatttcatgggtccatgtgttacaaaaataagaattggagcattaacttgaaattgtcttcaaggacttgctatgtgcacttcttcacaccggtacattctctgcatctgtttatatgaaataacacaagctaatctgtacctttatatatatgtgtgtgtacatatatacatgtataaactgtatagtgtacatgttaatgatttatcagtatgccccgaattcctagtgcagttctcattctccgcatgccctcagctgtggtcaggtgacttcctgtcccctggcagctctgctgagtccctgtgtttgagcctccagggagaagggtttgggctgcattcttcttatccccatgcacagaaacgctcagggtccccacgtgcctgttgtcctcccctctagtcctttgttccctttctgagcatgtggcccctcccccggctgtgaggggcagccgccttcctaggaaagtgtaatgcaggatcaagaccaccactgcatgtgtactgaaaactgcagacttctgcccgcttgggacagaacattgcatgtgaggtgggattatcaagtttcaaagacctgtgtcaaccatacagcaaaggcagaccccataacgacgtcccacaaaatggaaataaaacgcaaaatctctttagcattgtgtaaataaatctggatgtgtttaactttgtactggtaattttctgtatatttgcaatatttgggttagaaataaacagcctggactttggtacctaaaaaaaaaaaaaaaaaaaaaaaaaaaaaaaaaaaaaaaaaaaaaaaaaaa

SEQ ID NO: 34 - protein, 825 aa, Mus musculus
MERPRGAADGLLRWPLGLLLLLQLLPPAAVGQDRLDAPPPPAPPLLRWAGPVGVSWGLRAAAPGGPVPRAGRWRRGAPAEDQDCGRLPDFIAKLTNNTHQHVFDDLSGSVSLSWVGDSTGVILVLTTFQVPLVIVSFGQSKLYRSEDYGKNFKDITNLINNTFIRTEFGMAIGPENSGKVILTAEVSGGSRGGRVFRSSDFAKNFVQTDLPFHPLTQMMYSPQNSDYLLALSTENGLWVSKNFGEKWEEIHKAVCLAKWGPNNIIFFTTHVNGSCKADLGALELWRTSDLGKTFKTIGVKIYSFGLGGRFLFASVMADKDTTRRIHVSTDQGDTWSMAQLPSVGQEQFYSILAANEDMVFMHVDEPGDTGFGTIFTSDDRGIVYSKSLDRHLYTTTGGETDFTNVTSLRGVYITSTLSEDNSIQSMITFDQGGRWEHLRKPENSKCDATAKNKNECSLHIHASYSISQKLNVPMAPLSEPNAVGIVIAHGSVGDAISVMVPDVYISDDGGYSWAKMLEGPHYYTILDSGGIIVAIEHSNRPINVIKFSTDEGQCWQSYVFTQEPIYFTGLASEPGARSMNISIWGFTESFITRQWVSYTVDFKDILERNCEEDDYTTWLAHSTDPGDYKDGCILGYKEQFLRLRKSSVCQNGRDYVVAKQPSVCPCSLEDFLCDFGYFRPENASECVEQPELKGHELEFCLYGKEEHLTTNGYRKIPGDKCQGGMNPAREVKDLKKKCTSNFLNPTKQNSKSNSVPIILAIVGLMLVTVVAGVLIVKKYVCGGRFLVHRYSVLQQHAEADGVEALDSTSHAKSGYHDDSDEDLLE

SEQ ID NO: 35 - DNA, 6690 bp, Rattus norvegicus
agcggcgtctgcattcggcggcgatggagcggccccggggagctgcggacggccttttgcgctggccccttggcctcctcctgctcctgcagctgctgccgccagccgccgtcggccaggaccggctggacgcgccgccgcctcccgcgcctcctctgctgcgctgggccggtccggtcggggtgagctgggggctgcgcgccgccgcgcccgggggccctgtcccccgcgctggccgttggcgccgcggcgcgcctgcagaggatcaggactgcggccgcctcccggacttcatcgccaagctgaccaacaatacgcaccagcatgtctttgatgacctcagtggctcagtgtccttgtcctgggttggagacagcactggggttattctcgtcctgactactttccaagtgccactggtaattgtgagttttggacagtccaagttatatcgcagtgaggattacggaaagaattttaaggacattacgaatctcatcaataacaccttcattcggacggagttcggcatggctattggtcccgagaactctggaaaggtgatattaacagcagaggtgtctgggggaagccgaggaggaagagtgttcaggtcatcagactttgccaagaactttgtacagacagacctcccctttcatcctctgactcagatgatgtacagccctcagaattctgattacctgttagctctcagcactgaaaatggcctgtgggtgtccaagaattttggggaaaaatgggaagaaatccacaaagcagtatgtttggccaaatggggaccaaacaacatcatcttctttaccacccatgtgaatggctcctgcaaagctgatcttggtgccctggaattatggagaacatccgacttggggaaaaccttcaaaaccattggtgtgaaaatctactcatttggtcttgggggccgtttcctcttcgcctctgtgatggctgataaggatacaacaagaaggatccacgtgtcaacagaccaaggggacacatggagcatggcacagcttccctctgtgggacaggagcaattctattctattctggcagccaacgatgacatggtgttcatgcatgtagatgaacctggagatactggatttggtacaatctttacctctgatgaccgaggcattgtctactctaagtctctggacagacatctctataccaccacaggcggggagaccgactttaccaacgtgacttccctccgtggagtctatataacaagcacactctcagaagataattccattcagagcatgatcacttttgaccagggaggacggtgggagcatctgcagaagccggagaacagcaagtgcgatgctaccgcaaagaacaagaatgagtgcagccttcatatccatgcttcttacagcatctcccagaagctaaatgttccaatggccccactctccgagcccaatgccgtgggcatagtcatcgctcacggtagtgtgggagacgccatctcggtgatggtcccagacgtgtacatctcagatgatgggggttactcttgggcaaagatgctagaaggaccgcattattataccatcctggactctggaggcatcatcgtggccattgaacacagcaaccgtcctatcaatgtgattaagttctccacagacgaaggccagtgctggcagagctatgtgttcagtcaggagcccgtctacttcaccgggctggcttccgagccgggagccaggtccatgaacatcagcatctggggattcacggagtctttccttacccgccagtgggtctcctacacaattgatttcaaagacatccttgagcgcaactgtgaagagaatgactataccacatggctggcacactccactgaccccggagattacaaagatggctgcattttgggctataaagagcagttcctacggctacggaagtcatccgtctgtcagaatggtcgagactatgttgtggccaagcagccatccatctgtccctgttccctggaggacttcctctgtgactttggctacttccgtccagagaatgcctcagagtgtgtggagcagccggaactgaagggtcatgagttagagttctgtctgtatggcaaagaggagcacctgaccacaaatgggtaccggaaaatcccaggagacagatgccaagggggaatgaatccggcccgagaagtaaaagacttgaagaagaaatgcacaagcaacttcttaaaccccaagaagcagaattccaagtcgagttctgtccctatcatcctggccatcgtgggactgatgcttgtcactgtcgtagcaggagtcctcattgtgaagaagtatgtctgtggcggaaggttcctggtgcaccggtactcggtgcttcagcagcacgcagaggctgacggggtggaggctttggatacggcctcccacgctaaaagcggttatcacgatgattcagacgaggacctcctggaatagctcttctgaggagctgggactgagtgtgcatggagccacagtacctcctacattccctgaggctctgacttggggaaataaatcccccactgcgtcggccccagctctctttctgccgcttccacccacgcccaaaggacctacactaaagaaagcagggtgggggtggggaccccaactgatctcagttggctctgaaaagaggggggcaattttaaattctttaacatttttgtttcaatttctgtcttttgtaaaagtgtgggctttaggcttctctcggtttgttttgtatgttaagggacatgacttgggattcaaagggattgcttcgctgcccccactcttgtgtgtcctgtctggtgcccgccccagggcatctgccaactccaccatcaactcaactcccacatcatacctggtgctgtccctgggctctgctggtcatgcgaagccacctcagagaaggaaacagagggtgcagtgactggcgccgcctggccagcttccctccatccgtggacagccctgttcaaagagtaccgtgcaccagtgcaccagccctgctgcggcttcctcacccccaccccacccccaaccacgactctaaggcacagtgacttttcttttcccgtttaattaggcaatacccttgttaattgccctttggcaactaacttaatcatgtgcttccaacacactaaatagggaaaacttacagggcaatattattcttgtggcaagaaggagggagggacagaattgactagatctaacacctattaaagacaaaactggctgcttccaagctctttcaagctgtgctttgtggtgccagtggcgctgtcaaggcttggctcgaagcctgcccaggctccgccatccctttcctggagttcgttgttcagggcacaatgcggccaccagactgacagccatagctgtccatttttaccatcacccactgtaaaggctcagtgtcgagctaccgccatggctgcttattgcttcctctgaggttgctcccgttcagagaactgggactaggagtgttttcaggttgatggactttttcagactcaaagttacgttaccagactatcctcccacttcccccagtttagctgagttggttgttatccagaaccctttggctaaacccactgaaggcacgtttctagatggtgctgtgtgtggttgatacattgctttcgtgttttaaatttaaggaaattttctatctgatatttttccaaattaagtaagttgtagaatgagattggcggatgtgggggcggccatctggtaggggaaaggtacagagattcatctgaatacagcttagacattggctggacaggaagatggggtgtaggtctctggtgtccctccccctgctgtgctgatcagtccctccaactaccactaggacattctttagtaggtccatcttgctgtcacttcattctgtgactagaaagtcagtggaaagtaaatgtgccaaactagcttgtcagaattcgtgagcagatggccgagtccgctcctccccagagggaacaaacagtgtcagggaaagaggggaggaagaggagggagagccagagagatgctgacttgcagctccatgagtcatggaacgtggtctagaaaatgaagttagggtcgtttctgggaagagaagaccagaggatgctctagtctttagcttgttcactttgtaatgattctaaaataattccaaagatatagcagctcataagttatcaaagtccagcctcaccccctgccaaggggagagatgtgtttccactacagtctctcccagcacatccccaggttggtgtgggtaggtaggccagtgcatggtgatcttgcctagtgtctggatggacttcacactcgtccccagccatcaaagactggggtgctctgccgctaatggtttctgggatttctataggacatggccgctttgggatcatttctcttttgagcaccgcactattcttgccgtagatagcactcaggaacaaaggctgagtcagccacccagagtcctcggagctctgaaacacaagttatgcaaggtcctggctcctggcagcagagcttctctagggcgactggctggggccgagggcgctgaggcgttccttgcagagccattattttgacacctttttgttttcttttggagaacaagtttatctccctgcttcatgactgagacgaaaagtgatggtgattgggggcaccctctctcccttggagagtcactgttggatgagtcctctgcccttttgcttctctctgtagctcactgacttgggtactgctcctacaacagcttccagaagcattcacatgcacaaatgtgcgaccccagccagcagtttaagctccttaagctgttttaaaaaaacaaaaaaaccttctgctttggtttgtcctgtgcagccctggggcggcatcggcatcactggtgaacttaccactattctcggcactgtggtggccgatgggctggttctggagacttgggcagaaggatttagcattgttaggcagctcagacatattggaaagtccattgtggcctgagttttaaattccaatttttttttcttggcttgggaccttaggggcttggagttttcctgagaccggtgccgtggctgaatgcgctgtgctgtgaattgaatggaacaacgctttgaggcccttagagagtgactgcagctgcagggtgggctcaggagccttgggccccactgcctctgaacctgcatgcgtgcagtcccatgcgcctggctttcccctgcaccaggtgcaggctgctggtgtagtcccctgcctgctcacagctggtcccctagcctttcctctctggcattgtagtatcatgggccaccctcggctcctttattcatagtcaggcctatgaaccatgctggactaagtatgctgaactgtcccttcatcaggatactcgaggggagaaaggccattccaacaagcgccagagtgagagggctggcatggcatcgctcacttactgctctgcaggtaaagcacatgggtagagagcctccagcgctcggaagagccctgccactgcacactggcaaagggctcgctcacgtatcagtacagccctcccaactccacactcagcagggatgcactggactgctgatgtcgggcagggcacatctcagttttctatccccttcccaccagagggaaaaggagcatgaactttaatgtcttttggaacaaaaccaaagtttaaggtttgccatatgatcagtgacagggaagcccagagccggccagcaggtgccagattttctgtcccactggccagtcctgcaccaggtgcaggctgctctgggcttgttgaaaccacagatgcaggtgggtttcgtggttccattcgttatatcattaagaattggagcattaacttgaaactgtcttcaaagacttgctgtgtgcactgtctcacactactacattctctgcatctgtttatataagataacacaagccaatctgtacctttatatatatatatgtgtgtgtgtacatatatacatgtataaactgtatagtgtacatgttaatgacttatcagtatgccccgaatttttagtgcagttctcatcctccgcatgccctcagctgtggtcaggtgacctcctgtcccctggtggctcagccgagtccctgtgtttgagcctccagggagaagggttggggctgcattctccttatccccatgcacagaaacgctcagggtccccacgtgcctgttgtcctcccctctggtcttctgttccctttctgagcatgtggcccttcccccggctgtgaggggcggctgccttcctgggagagcaggatcaagaccaccactgcatgtgtactgaaaactccggacttctgccctcttgggacagaacattgcatgtgaggtgggattatcaagtttcaaaaacctgtgtcaattatacagcaaagccagaccacataacgacatcccacaaaatggaaataaaacgcaaaatctctttagcattgtgtaaataaatctggacgtgtttaactttgtactggtaattttctgtatatttgcaatatttgggttagaaataaaacagcctggactttggtacctaaccta

SEQ ID NO: 36 - protein, 825 aa, Rattus norvegicus
MERPRGAADGLLRWPLGLLLLLQLLPPAAVGQDRLDAPPPPAPPLLRWAGPVGVSWGLRAAAPGGPVPRAGRWRRGAPAEDQDCGRLPDFIAKLTNNTHQHVFDDLSGSVSLSWVGDSTGVILVLTTFQVPLVIVSFGQSKLYRSEDYGKNFKDITNLINNTFIRTEFGMAIGPENSGKVILTAEVSGGSRGGRVFRSSDFAKNFVQTDLPFHPLTQMMYSPQNSDYLLALSTENGLWVSKNFGEKWEEIHKAVCLAKWGPNNIIFFTTHVNGSCKADLGALELWRTSDLGKTFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSTLSEDNSIQSMITFDQGGRWEHLQKPENSKCDATAKNKNECSLHIHASYSISQKLNVPMAPLSEPNAVGIVIAHGSVGDAISVMVPDVYISDDGGYSWAKMLEGPHYYTILDSGGIIVAIEHSNRPINVIKFSTDEGQCWQSYVFSQEPVYFTGLASEPGARSMNISIWGFTESFLTRQWVSYTIDFKDILERNCEENDYTTWLAHSTDPGDYKDGCILGYKEQFLRLRKSSVCQNGRDYVVAKQPSICPCSLEDFLCDFGYFRPENASECVEQPELKGHELEFCLYGKEEHLTTNGYRKIPGDRCQGGMNPAREVKDLKKKCTSNFLNPKKQNSKSSSVPIILAIVGLMLVTVVAGVLIVKKYVCGGRFLVHRYSVLQQHAEADGVEALDTASHAKSGYHDDSDEDLLE

(19) United States
(12) Patent Application Publication
(10) Pub. No.: US 2012/0039865 A1
(43) Pub. Date: Feb. 16, 2012

(54) IDENTIFICATION OF SORTILIN AS A NEURONAL RECEPTOR FOR THE FRONTOTEMPORAL DEMENTIA PROTEIN, PROGRANULIN

(75) Inventors: Stephen M. Strittmatter, Guilford, CT (US); Fenghua Hu, Ithaca, NY (US); Thihan Padukkavidana, New Haven, CT (US)

(73) Assignee: Yale University, New Haven, CT (US)

(21) Appl. No.: 13/059,849

(22) PCT Filed: Aug. 19, 2009
(86) PCT No.: PCT/US2009/054347

§ 371 Date: Oct. 21, 2011

Related U.S. Application Data
(60) Provisional application No. 61/189,480, filed on Aug. 19, 2008.

(51) Int. Cl. A61K 39/395; A61K 31/7088; A61K 38/00; A61K 38/12; C12N 5/02; G01N 33/566

(52) U.S. Cl. 424/130.1; 435/375; 436/501; 514/1.1; 514/21.1; 514/44 R; 514/44 A

(57) ABSTRACT

The present invention is related to methods for identifying compounds that modulate the interaction between progranulin and sortilin. The present invention is also related to methods for modulating the interaction between progranulin and sortilin, the activity of progranulin or sortilin, or the activity of progranulin and sortilin. The present invention also includes methods of treating or preventing a disease, disorder, or condition by modulating the interaction between progranulin and sortilin, the activity of progranulin or sortilin, or the activity of progranulin and sortilin.

### BACKGROUND OF THE INVENTION

1. Field of the Invention

This invention relates generally to the fields of neurobiology, neurology, and pharmacology. The invention is based on the surprising discovery that sortilin functions as a receptor for progranulin. Specifically, the present invention is related to methods for identifying compounds that modulate the interaction between progranulin and sortilin. The present invention is also related to methods for modulating the interaction between progranulin and sortilin, the activity of progranulin or sortilin, or the activity of progranulin and sortilin. The present invention also includes methods of treating or preventing a disease, disorder, or condition by modulating the interaction between progranulin and sortilin, the activity of progranulin or sortilin, or the activity of progranulin and sortilin.

2. Background

Dementia afflicts a large and rapidly growing number of individuals and their families. A majority of cases are caused by Alzheimer's Disease (AD), but substantial percentages are attributed to other causes. Frontotemporal dementia (FTD) is the third most common cause of neurodegenerative dementia, after AD and Diffuse Lewy Body Disease (Neary, D., Snowden, J. & Mann, D., Lancet Neurol. 4:771-780 (2005); Cruts, M. & Van Broeckhoven, C., Trends Genet. 24:186-194 (2008)). The clinical features of FTD include memory deficits, behavioral abnormalities, personality changes, and language impairments (Cruts, M. & Van Broeckhoven, C., Trends Genet. 24:186-194 (2008); Neary, D., et al., Neurology 51:1546-1554 (1998); Ratnavalli, E., Brayne, C., Dawson, K. & Hodges, J. R., Neurology 58:1615-1621 (2002)).

A substantial portion of FTD cases are inherited in an autosomal dominant fashion, but even in one family symptoms can span a spectrum from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain. Historically, the first descriptions of FTD recognized the presence of intraneuronal accumulations of hyperphosphorylated Tau protein in neurofibrillary tangles or Pick bodies. A causal role for the microtubule associated protein Tau was supported by the identification of mutations in the gene encoding the Tau protein in several families (Hutton, M., et al., Nature 393:702-705 (1998). However, the majority of FTD brains show no accumulation of hyperphosphorylated Tau but do exhibit immunoreactivity to ubiquitin (Ub) and TAR DNA binding protein (TDP43) (Neumann, M., et al., Arch. Neurol. 64:1388-1394 (2007)). A majority of those FTD cases with Ub inclusions (FTD-U) were shown to carry mutations in the Progranulin gene on chromosome 17 (Baker, M., et al., Nature 442:916-919 (2006); Cruts, M., et al., Nature 442:920-924 (2006)). PGRN mutations result in haploinsufficiency (Baker, M., et al., Nature 442:916-919 (2006); Cruts, M., et al., Nature 442:920-924 (2006)) and are known to be present in nearly 50% of familial FTD cases, making PGRN mutation a major genetic contributor to FTD (Cruts, M. & Van Broeckhoven, C., Trends Genet. 24:186-194 (2008); Le Ber, I., et al., Brain 129:3051-3065 (2006)). The loss-of-function heterozygous character of PGRN mutations implies that in healthy individuals, PGRN expression plays a dose-dependent, critical role in protecting healthy individuals from the development of FTD.

Pathological TDP43 aggregates occur in certain degenerative diseases other than FTD, most notably in sporadic ALS (amyotrophic lateral sclerosis) (Mackenzie, I. R., et al., Ann. Neurol. 61:427-434 (2007); Sreedharan, J., et al., Science 319:1668-1672 (2008); Kabashi, E., et al., Nat. Genet. 40:572-574 (2008)). Though genetic studies have not revealed PGRN mutation as a common etiology for ALS, the shared TDP43 pathology implies that further understanding of a PGRN-dependent pathway may have relevance for both ALS and FTD.

PGRN (proepithelin, granulin-epithelin precursor, PC-cell-derived growth factor, acrogranin) encodes a 68.5 kDa secreted glycoprotein that has 7.5 repeats of smaller granulin motifs, ranging from 6-25 kDa, which can be proteolytically cleaved from the precursor PGRN (He, Z. & Bateman, A., J. Mol. Med. 81:600-612 (2003)). In non-neuronal cells, PGRN has been associated with a variety of events, such as cell cycle regulation and cell motility (He, Z. & Bateman, A., J. Mol. Med. 81:600-612 (2003); Monami, G., et al., Cancer Res. 66:7103-7110 (2006)), wound repair, inflammation (Zhu, J., et al., Cell 111:867-878 (2002)), induction of growth factors such as vascular endothelial growth factor (VEGF) (Tangkeangsirisin, W. & Serrero, G, Carcinogenesis 25:1587-1592 (2004)), and tumorigenesis (He, Z. & Bateman, A., J. Mol. Med. 81:600-612 (2003); Monami, G., et al., Cancer Res. 66:7103-7110 (2006); Serrero, G., Biochem. Biophys. Res. Commun. 308:409-413 (2003); Lu, R. & Serrero, G., Proc. Natl. Acad. Sci. U.S.A. 98:142-147 (2001); Liau, L. M., et al., Cancer Res. 60:1353-1360 (2000)). Both the uncleaved PGRN protein and the derivative GRN peptides have activity in these assays (Zhu, J., et al., Cell 111:867-878 (2002), and in some cases their actions oppose one another. The cell surface receptor basis for PGRN and GRN action has not been described for any cell type.

Despite the strong association of FTD with PGRN mutations, essentially nothing is known about the mechanism of PGRN action in the brain, except that it is required to prevent FTD. Therefore, there is a need to identify targets for FTD treatment and prevention, as well as, a need to identify targets for ALS treatment and prevention. The present application describes the identification of one such target, which is the high-affinity receptor through which PGRN signals in the brain, sortilin. Thus, there is a need for molecules that could modulate the PGRN and sortilin pathway. The molecules would be useful for treating or preventing diseases such as, but not limited to, FTD and ALS.

### BRIEF SUMMARY OF THE INVENTION

The present invention is based on the surprising discovery that sortilin functions as a receptor for progranulin and is directed to new and useful methods for screening for compounds that modulate the interaction between sortilin and progranulin and to methods for using those compounds to modulate the sortilin and progranulin interaction. Additional methods are described herein.

In one embodiment, the present invention provides a method for identifying a compound that modulates the interaction of sortilin and progranulin comprising (a) mixing a compound with sortilin and progranulin; and (b) measuring the interaction of sortilin and progranulin in the presence of said compound as compared to the interaction of sortilin to progranulin in the absence of said compound. In a further embodiment, the method for identifying a compound that modulates the interaction of sortilin and progranulin comprises comparing the interaction of sortilin and progranulin in the presence of said compound to the interaction of sortilin and progranulin in the absence of said compound.

In another embodiment, the present invention provides a method of promoting cell survival by modulating the interaction between progranulin and sortilin comprising (a) contacting a cell with a compound; and (b) modulating the interaction between progranulin and sortilin. In a particular embodiment, the compound promotes or mimics the binding of progranulin to sortilin.

In one embodiment, the present invention provides a method for increasing levels of progranulin in a mammal comprising administering to said mammal a compound that modulates the interaction between progranulin and sortilin. In another embodiment, the present invention provides a method for increasing levels of progranulin in a mammal comprising administering to said mammal a compound that modulates the activity of sortilin.

In some embodiments, a method for modulating the interaction between progranulin and sortilin in a mammal is provided. In a particular embodiment, the present invention provides a method for modulating the interaction between progranulin and sortilin in a mammal comprising administering to said mammal a therapeutically effective amount of a compound capable of modulating said interaction. In another embodiment, the present invention provides a method for modulating the activity of sortilin in a mammal comprising administering to said mammal a therapeutically effective amount of a compound capable of modulating the interaction between sortilin and progranulin. In one embodiment, the present invention provides a method for modulating the activity of progranulin in a mammal comprising administering to said mammal a therapeutically effective amount of a compound capable of modulating the interaction between sortilin and progranulin. In a particular embodiment, the modulation of the interaction between sortilin and progranulin reduces signs or symptoms of a disease, disorder, or condition in a mammal in need thereof.

In some embodiments, the present invention provides a method of treating or preventing a disease, disorder, or condition comprising administering to a mammal in need thereof a therapeutically or prophylactically effective amount of a compound, wherein said compound is selected from the group consisting of (a) a compound capable of modulating the interaction between sortilin and a progranulin; (b) a compound capable of modulating the activity of sortilin, and a compound identified according to the methods of screening provided herein. In particular embodiments, the disease, disorder, or condition treated or prevented in the methods of the invention is selected from the group consisting of frontotemporal dementia (FTD) and amyotrophic lateral sclerosis (ALS). In a preferred embodiment, the disease, disorder, or condition is frontotemporal dementia (FTD).

In some embodiments of the methods of the invention, modulating the interaction between progranulin and sortilin, the activity of sortilin, or the activity of progranulin is inhibition of the interaction between progranulin and sortilin, the activity of sortilin, or the activity of progranulin. In other embodiments of the methods of the invention, modulating the interaction between progranulin and sortilin, the activity of sortilin, or the activity of progranulin is enhancement of the interaction between progranulin and sortilin, the activity of sortilin, or the activity of progranulin.

In particular embodiments, the mammal treated in the methods of the invention is a human.

In some embodiments, the compounds used in the methods of the invention are selected from the group consisting of: (a) an antibody, or antigen-binding fragment thereof; (b) a polypeptide; (c) a polynucleotide; and (d) a combination of one or more of these. In particular embodiments, the antibody, or antigen-binding fragment thereof, specifically binds a sortilin polypeptide. In other embodiments, the antibody, or antigen-binding fragment thereof, specifically binds a sortilin polypeptide that comprises an amino acid sequence selected from the group consisting of SEQ ID NO:21, SEQ ID NO:34, and SEQ ID NO:36. In some embodiments, the antibody, or antigen-binding fragment thereof, is selected from the group consisting of a polyclonal antibody, a monoclonal antibody, a Fab fragment, a Fab′ fragment, a F(ab′)2 fragment, an Fv fragment, an Fd fragment, a diabody, and a single-chain antibody.

In some embodiments, the polypeptides used in the methods of the invention comprise a polypeptide selected from the group consisting of: (a) a sortilin polypeptide; (b) a fragment of a sortilin polypeptide; (c) a polypeptide selected from the group consisting of SEQ ID NO:21, SEQ ID NO:34, and SEQ ID NO:36; (d) a progranulin polypeptide; (e) a fragment of a progranulin polypeptide; (f) a polypeptide selected from the group consisting of SEQ ID NO:30 and SEQ ID NO:32; (g) a progranulin polypeptide comprising amino acids 577-593 of SEQ ID NO:30 or amino acids 584-602 of SEQ ID NO:32; (h) a polypeptide that is 70%, 75%, 80%, 85%, 90%, 95%, or 100% identical to a reference amino acid sequence selected from the group consisting of: (i) SEQ ID NO: 10, (ii) SEQ ID NO: 11, (iii) SEQ ID NO:12, (iv) SEQ ID NO:13, (v) SEQ ID NO:14, (vi) SEQ ID NO:SEQ ID NO:15, (vii) SEQ ID NO:16, (viii) SEQ ID NO:17, (ix) SEQ ID NO:18, (x) SEQ ID NO:19, (xi) SEQ ID NO:21, (xii) SEQ ID NO:30, (xiii) SEQ ID NO:32, (xiv) SEQ ID NO:34, and (xv) SEQ ID NO:36; and (i) a variant, derivative, or analog of any one of these.

In particular embodiments, the polypeptide used in the methods of the invention is a fusion protein comprising a heterologous polypeptide or a polymer. In some embodiments, the heterologous polypeptide is selected from the group consisting of: (a) serum albumin; (b) an Fc region selected from the group consisting of an IgA Fc region, an IgD Fc region, an IgG Fc region, an IgE Fc region, and an IgM Fc region; (c) a signal peptide; (d) a polypeptide tag selected from the group consisting of a FLAG tag; a Strep tag; a poly-histidine tag; a VSV-G tag; an influenza virus hemagglutinin (HA) tag; and a c-Myc tag; and (e) a combination of one or more of these. In other embodiments, the polymer is selected from the group consisting of: (a) a polyalkylene glycol; (b) a sugar polymer; (c) a polypeptide; and (d) a combination one or more of these. In particular embodiments, the polypeptide used in the methods of the invention is conjugated to 1, 2, 3, or 4 polymers. In some embodiments, the total molecular weight of the polymers is from 5,000 Da to 100,000 Da.

In some embodiments, the polypeptide used in the methods of the invention is a cyclic polypeptide. In particular embodiments, the cyclic polypeptide further comprises a first molecule linked at the N-terminus and a second molecule linked at the C-terminus, wherein said first molecule and said second molecule are joined to each other to form said cyclic molecule. In other embodiments, the first and second molecules of the cyclic polypeptide are selected from the group consisting of a biotin molecule, a cysteine residue, and an acetylated cysteine residue.

In some embodiments, the polynucleotide used in the methods of the present invention comprises an isolated polynucleotide selected from the group consisting of: (a) an antisense polynucleotide; (b) a ribozyme; (c) a small interfering RNA (siRNA); (d) a small-hairpin RNA (shRNA); and (e) a combination of one or more of these. In particular embodiments, the polynucleotide used in the methods of the invention comprises a sortilin nucleic acid or a progranulin nucleic acid. In other embodiments, the polynucleotide used in the methods of the invention is an antisense polynucleotide comprising at least 10 bases complementary to the coding region of sortilin mRNA.

### BRIEF DESCRIPTION OF THE DRAWINGS/FIGURES

FIG. 1A depicts an α-AP immunoblot of the conditioned medium from HEK293T cells transfected with an AP or an AP-PGRN expression vector.

FIG. 1B depicts a Coomassie Blue stained gel (“Total Protein” lane) and an α-Flag immunoblot (“α-Flag Blot” lane) of Flag-tagged PGRN (Flag-PGRN) isolated from HEK293T cells transfected with a Flag-tagged PGRN expression vector.

FIG. 1C depicts rat cortical neurons incubated with 10 nM AP-PGRN (top panel), 10 nM AP-PGRN and 100 nM Flag-PGRN (middle panel), or 10 nM AP (bottom panel). Binding of AP-PGRN was detected as a dark reaction product produced by AP (alkaline phosphatase).

FIG. 1D is a graph depicting the saturation analysis of binding of AP (diamonds) or AP-PGRN (squares) to rat cortical neurons. The binding of AP or AP-PGRN, measured by the optical density (OD units) of AP ligand bound, was determined as a function of ligand concentration (nM).

FIG. 2A depicts the binding of AP-PGRN to COS-7 cells transfected with empty vector or mouse cDNAs. Binding of AP-PGRN was detected as a dark reaction product produced by AP.

FIG. 2B is a graph depicting the saturation analysis of binding of AP-PGRN to COS-7 cells transfected with the PRGN-R, sortilin. The binding of AP-PGRN, measured by the optical density (OD units) of AP ligand bound, was determined as a function of ligand concentration (nM).

FIG. 3A depicts the binding of 5 nM AP-PGRN to mouse cortical neurons (left panel) and the displacement of binding of 5 nM AP-PGRN to mouse cortical neurons by 100 nM unlabeled PGRN (right panel).

FIG. 3B depicts the binding of 2 nM AP-PGRN to mouse cortical neurons from sortilin −/− mice (left panel) and sortilin +/− mice (right panel).

FIG. 4A depicts the specificity of binding of 20 nM AP-PGRN to COS-7 cells transfected with a sortilin (Sort1) expression vector.

FIG. 4B depicts the specificity of binding of 120 nM AP-PGRN to COS-7 cells transfected with a SorLA expression vector.

FIG. 4C depicts the specificity of binding of 120 nM AP-PGRN to COS-7 cells transfected with a SorCS 1 expression vector.

FIG. 5A depicts an α-PGRN immunoblot of serum isolated from WT (+/+) or Sortilin −/− mice. Lanes 1, 3, and 5 represent serum isolated from three independent WT (+/+) animals, and lanes 2 and 4 represent serum isolated from two independent Sortilin (−/−) mice

FIG. 5B is a graph depicting the quantitation of serum PGRN levels in WT (+/+) mice compared to sortilin −/− mice. Data are plotted as mean±sem.

FIG. 6A depicts the protective effect of PGRN on cortical neurons as measured by an apoptotic indicator, cleaved caspase 3. Fixed cells were stained with anti-cleaved caspase 3 antibody to detect apoptotic cells (shown in white) and with DAPI to detect all nuclei (shown in gray).

FIG. 6B is a graph depicting the apoptosis of the cells shown in FIG. 6A. Apoptosis was measured as the percent of cleaved caspase 3 plus vehicle. The Apoptosis value=(number of cleaved caspase 3 positive cells/number of DAPI positive nuclei)*100. Data are plotted as mean±sem.

FIG. 6C depicts the effect of PGRN on cortical neurite outgrowth as measured by the neuron-specific marker βIII tubulin. Cortical neurons isolated from E18 rat embryos and incubated in vehicle (upper panel) showed no significant difference in βIII tubulin staining as compared to cortical neurons incubated in 100 nM PGRN (lower panel).

FIG. 6D is a graph depicting neurite outgrowth as function of PGRN concentration. Neurite outgrowth in cultures, as in FIG. 6C, was measured (μm/neuron) and plotted versus PGRN concentration (nM). Data are plotted as mean±sem.

### DETAILED DESCRIPTION OF THE INVENTION

### Definitions and General Techniques

Unless defined otherwise, all technical and scientific terms used herein have the same meaning as commonly understood by one of ordinary skill in the art to which this invention belongs. In case of conflict, the present application including the definitions will control. Also, unless otherwise required by context, singular terms shall include pluralities and plural terms shall include the singular. All publications, patents and other references mentioned herein are incorporated by reference in their entireties for all purposes.

Although methods and materials similar or equivalent to those described herein can be used in practice or testing of the present invention, suitable methods and materials are described below. The materials, methods and examples are illustrative only, and are not intended to be limiting. Other features and advantages of the invention will be apparent from the detailed description and from the claims.

In order to further define this invention, the following terms and definitions are herein provided.

Throughout this specification and claims, the word “comprise,” or variations such as “comprises” or “comprising,” will be understood to imply the inclusion of a stated integer or group of integers but not the exclusion of any other integer or group of integers.

It is to be noted that the term “a” or “an” entity, refers to one or more of that entity; for example, “an immunoglobulin molecule,” is understood to represent one or more immunoglobulin molecules. As such, the terms “a” (or “an”), “one or more,” and “at least one” can be used interchangeably herein.

As used herein, the term “consists of,” or variations such as “consist of or “consisting of,” as used throughout the specification and claims, indicate the inclusion of any recited integer or group of integers, but that no additional integer or group of integers may be added to the specified method, structure or composition.

As used herein, the term “consists essentially of,” or variations such as “consist essentially of or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, “progranulin” and “PGRN” each mean progranulin. As described below, progranulin comprises 7.5 repeats of smaller granulin (epithelin) motifs known as GRN A-G and paragranulin (Van Damme, P., et al., J. Cell Biol. 181:37-41 (2008)). In the methods of the invention, the term “progranulin” or “PGRN” includes granulins, e.g., GRN A-G and paragranulin, that bind to sortilin.

As used herein, “sortilin,” “sortilin 1,” “sort1” and “PGRN-R” each mean sortilin.

As used herein, “antibody” means an intact immunoglobulin, or an antigen-binding fragment thereof. Antibodies of this invention can be of any isotype or class (e.g., M, D, G, E and A) or any subclass (e.g., G1-4, A1-2) and can have either a kappa (κ) or lambda (λ) light chain.

As used herein, “Fc” means a portion of an immunoglobulin heavy chain that comprises one or more heavy chain constant region domains, CH1, CH2, and CH3. For example, a portion of the heavy chain constant region of an antibody that is obtainable by papain digestion.

As used herein, “humanized antibody” means an antibody in which at least a portion of the non-human sequences are replaced with human sequences. Examples of how to make humanized antibodies may be found in U.S. Pat. Nos. 6,054,297, 5,886,152, and 5,877,293.

As used herein, “chimeric antibody” means an antibody that contains one or more regions from a first antibody and one or more regions from at least one other antibody. The first antibody and the additional antibodies can be from the same or different species.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide for use in the methods of the invention may be of a size of about 3 or more, 4 or more, 5 or more, 6 or more, 7 or more, 8 or more, 9 or more, 10 or more, 15 or more, 20 or more, 25 or more, 30 or more, 40 or more, 50 or more, 75 or more, 100 or more, 200 or more, 300 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposes of the invention, as are native or recombinant polypeptides which have been separated, fractionated, or partially or substantially purified by any suitable technique.

A “polypeptide fragment” refers to a short amino acid sequence of a larger polypeptide. Protein fragments may be “free-standing,” or comprised within a larger polypeptide of which the fragment forms a part. Representative examples of polypeptide fragments of the invention, include, for example, fragments comprising about 3 amino acids, about 4 amino acids, about 5 amino acids, about 6 amino acids, about 7 amino acids, about 8 amino acids, about 9 amino acids, about 10 amino acids, about 15 amino acids, about 20 amino acids, about 30 amino acids, about 40 amino acids, about 50 amino acids, about 60 amino acids, about 70 amino acids, about 80 amino acids, about 90 amino acids, and about 100 amino acids or more in length.

The terms “fragment,” “variant,” “derivative” and “analog” when referring to a polypeptide for use in the methods of the present invention include any polypeptide which retains at least some biological activity. Polypeptides as described herein may include fragment, variant, or derivative molecules therein without limitation, so long as the polypeptide still serves its function. Polypeptides and polypeptide fragments may include proteolytic fragments, deletion fragments and in particular, fragments which more easily reach the site of action when delivered to an animal. Polypeptide fragments further include any portion of the polypeptide which comprises an antigenic or immunogenic epitope of the native polypeptide, including linear as well as three-dimensional epitopes. Polypeptides and polypeptide fragments may comprise variant regions, including fragments as described above, and also polypeptides with altered amino acid sequences due to amino acid substitutions, deletions, or insertions. Variants may occur naturally, such as an allelic variant. By an “allelic variant” is intended alternate forms of a gene occupying a given locus on a chromosome of an organism. Genes II, Lewin, B., ed., John Wiley & Sons, New York (1985). Non-naturally occurring variants may be produced using art-known mutagenesis techniques. Polypeptides and polypeptide fragments may comprise conservative or non-conservative amino acid substitutions, deletions or additions. Polypeptides and polypeptide fragments may also include derivative molecules. Variant polypeptides may also be referred to herein as “polypeptide analogs.” As used herein a “derivative” of a polypeptide or a polypeptide fragment refers to a subject polypeptide having one or more residues chemically derivatized by reaction of a functional side group. Also included as “derivatives” are those peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

As used herein the term “disulfide bond” includes the covalent bond formed between two sulfur atoms. The amino acid cysteine comprises a thiol group that can form a disulfide bond or bridge with a second thiol group.

As used herein, “fusion protein” means a protein comprising a first polypeptide linearly connected, via peptide bonds, to a second, polypeptide. The first polypeptide and the second polypeptide may be identical or different, and they may be directly connected, or connected via a peptide linker (see below).

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide can contain the nucleotide sequence of the full-length cDNA sequence, including the untranslated 5′ and 3′ sequences and the coding sequence(s). A polynucleotide may comprise a conventional phosphodiester bond or a non-conventional bond (e.g., an amide bond, such as found in peptide nucleic acids (PNA)). The polynucleotide can be composed of any polyribonucleotide or polydeoxyribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding a polypeptide or polypeptide fragment for use in the methods of the invention contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides, e.g., in the form of messenger RNA (mRNA). Isolated polynucleotides or nucleic acids further include such molecules produced synthetically. In addition, a polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid may encode heterologous coding regions, either fused or unfused to a nucleic acid encoding a polypeptide or polypeptide fragment for use in the methods of the present invention. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

As used herein the term “engineered” includes manipulation of nucleic acid or polypeptide molecules by synthetic means (e.g., by recombinant techniques, in vitro peptide synthesis, by enzymatic or chemical coupling of peptides or some combination of these techniques).

As used herein, the terms “linked,” “fused” and “fusion” are used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two ore more segments that correspond to polypeptides encoded by the original ORFs (which segments are not normally so joined in nature.) Although the reading frame is thus made continuous throughout the fused segments, the segments may be physically or spatially separated by, for example, in-frame linker sequence.

A “linker” sequence is a series of one or more amino acids separating two polypeptide coding regions in a fusion protein. A typical linker comprises at least 5 amino acids. Additional linkers comprise at least 10 or at least 15 amino acids. In certain embodiments, the amino acids of a peptide linker are selected so that the linker is hydrophilic. The linker (Gly-Gly-Gly-Gly-Ser)₃ (G₄S)₃ (SEQ ID NO:1) is a preferred linker that is widely applicable to many antibodies as it provides sufficient flexibility. Other linkers include (Gly-Gly-Gly-Gly-Ser)₂ (G4S)₂ (SEQ ID NO:2); Glu Ser Gly Arg Ser Gly Gly Gly Gly Ser Gly Gly Gly Gly Ser (SEQ ID NO:3); Glu Gly Lys Ser Ser Gly Ser Gly Ser Glu Ser Lys Ser Thr (SEQ ID NO:4); Glu Gly Lys Ser Ser Gly Ser Gly Ser Glu Ser Lys Ser Thr Gln (SEQ ID NO:5); Glu Gly Lys Ser Ser Gly Ser Gly Ser Glu Ser Lys Val Asp (SEQ ID NO:6); Gly Ser Thr Ser Gly Ser Gly Lys Ser Ser Glu Gly Lys Gly (SEQ ID NO:7); Lys Glu Ser Gly Ser Val Ser Ser Glu Gln Leu Ala Gln Phe Arg Ser Leu Asp (SEQ ID NO:8); and Glu Ser Gly Ser Val Ser Ser Glu Glu Leu Ala Phe Arg Ser Leu Asp (SEQ ID NO:9). Examples of shorter linkers include fragments of the above linkers, and examples of longer linkers include combinations of the linkers above, combinations of fragments of the linkers above, and combinations of the linkers above with fragments of the linkers above.

In the context of polypeptides, a “linear sequence” or a “sequence” is an order of amino acids in a polypeptide in an amino to carboxyl terminal direction in which residues that neighbor each other in the sequence are contiguous in the primary structure of the polypeptide.

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes, without limitation, transcription of the gene into messenger RNA (mRNA), transfer RNA (tRNA), small hairpin RNA (shRNA), small interfering RNA (siRNA) or any other RNA product, and the translation of such mRNA into polypeptide(s), as well as any processes which regulate either transcription or translation. If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, integrated into a chromosome or present in a transfection vector that is not integrated into the genome. The expression of the gene is either completely or partially inhibited. RNAi may also be considered to inhibit the function of a target RNA; the function of the target RNA may be complete or partial.

As used herein, the terms “treat” or “treatment” refer to both therapeutic treatment and prophylactic or preventative measures, wherein the object is to prevent or slow down (lessen) an undesired physiological change or disorder, such as the progression of FTD. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

As used herein, a “therapeutically effective amount” refers to an amount effective, at dosages and for periods of time necessary, to achieve the desired therapeutic result. A therapeutic result may be, e.g., lessening of symptoms, prolonged survival, improved mobility, and the like. A therapeutic result need not be a “cure”.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

As used herein, “modulate” means a change or changes in the activity of PGRN, sortilin, or the PGRN-sortilin interaction, so long as the change or changes in activity facilitate(s) the treatment or prevention of FTD or ALS. Thus, the modulation of PGRN, sortilin, or the PGRN-sortilin interaction may be a decrease or inhibition in the activity of PGRN, sortilin, or the PGRN-sortilin interaction, or the modulation of PGRN, sortilin, or the PGRN-sortilin interaction may be an increase or enhancement in the activity of PGRN, sortilin, or the PGRN-sortilin interaction.

As used herein, a “compound” means any type of compound that is capable of modulating a change or changes in the activity of PGRN, sortilin, or the PGRN-sortilin interaction. A compound includes any of the classes of compounds recited herein and includes, but is not limited to, proteins, polypeptides, peptides, antibodies or antigen-binding fragments thereof, polynucleotides, antisense polynucleotides, siRNA, or aptamers. A compound also includes a small molecule, e.g., an amino acid, a nucleotide, an organic or inorganic compound. Compounds can be naturally occurring, e.g., a natural product, synthetic, or can include both natural and synthetic components.

### Progranulins

Progranulin (PGRN) is variously referred to in the literature as proepithelin, granulin-epithelin precursor, PC (prostate cancer) cell-derived growth factor (PCDGF), and acrogranin. PGRN is a 593 amino acid protein that encodes a 68.5 kDa secreted glycoprotein that has 7.5 repeats of smaller granulin (epithelin) motifs, ranging from 6-25 kDa, which can be proteolytically cleaved from the precursor PGRN (He, Z. & Bateman, A., J. Mol. Med. 81:600-612 (2003)). The PGRN cleavage products are known as GRN A-G and paragranulin (Van Damme, P., et al., J. Cell Biol. 181:37-41 (2008)). PGRN is cysteine-rich and each granulin motif contains highly conserved tandem repeats of a rare 12-cysteine motif (Ahmed, Z., et al., J. Neuroinf 4:7 (2007); Bhandari V., Palfree R. G. & Bateman A., Proc. Natl. Acad. Sci. U.S.A. 89:1715-1719 (1992); Avrova, A. O., et al., Mol. Plant Microbe Interact. 12:1114-1119 (1999)). Domains of human PGRN are shown in Table 1, while nucleotide and protein sequences of human and mouse PGRN are provided in SEQ ID NOs: 29 and 30 and in SEQ ID NOs:31 and 32, respectively.

TABLE 1
Human PGRN Domains

| PGRN Domain | PGRN Sequence (SEQ ID NO: 30) |
|---|---|
| Signal sequence | 1-MWTLVSWVALTAGLVAG-17 (SEQ ID NO: 10) |
| Paragranulin (P) | 20-CPDGQFCPVACCLDPGGASYSCCRP-44 (SEQ ID NO: 11) |
| Granulin 7 (G) | 61-CQVDAHCSAGHSCIFTVSGTSSCCPFPEAVACGDGHHCCPRGF |
| | HCSADGRSC-112 (SEQ ID NO: 12) |
| Granulin 6 (F) | 126-CPDSQFECPDFSTCCVMVDGSWGCCPMPQASCCEDRVHCCP |
| | HGAFCDLVHTRC-178 (SEQ ID NO: 13) |
| Granulin 2 (B) | 208-CPDARSRCPDGSTCCELPSGKYGCCPMPNATCCSDHLHCC |
| | PQDTVCDLIQSKC-260 (SEQ ID NO: 14) |
| Granulin 1 (A) | 284-CDMEVSCPDGYTCCRLQSGAWGCCPFTQAVCCEDHIHCCP |
| | AGFTCDTQKGTC-335 (SEQ ID NO: 15) |
| Granulin 3 (C) | 366-CDNVSSCPSSDTCCQLTSGEWGCCPIPEAVCCSDHQHCCPQG |
| | YTCVAEGQC-416 (SEQ ID NO: 16) |
| Granulin 4 (D) | 444-CDQHTSCPVGQTCCPSLGGSWACCQLPHAVCCEDRQHCCPAG |
| | YTCNVKARSC-495 (SEQ ID NO: 17) |
| Granulin 5 (E) | 521-CGEGHFCHDNQTCCRDNRQGWACCPYRQGVCCADRRHCCPAG |
| | FRCAARGTKC-572 (SEQ ID NO: 18) |
| C-terminus | 573-LRREAPRWDAPLRDPALRQLL-593 (SEQ ID NO: 19) |

As one of skill in the art will appreciate, the beginning and ending residues of the domains listed above may vary depending upon the computer modeling program used or the method used for determining the domain.

PGRN is widely expressed and in non-neuronal cells, has been associated with a variety of events, such as cell cycle regulation and cell motility (He, Z. & Bateman, A., J. Mol. Med. 81:600-612 (2003); Monami, G., et al., Cancer Res. 66:7103-7110 (2006)), wound repair, inflammation (Zhu, J., et al., Cell 111:867-878 (2002)), induction of growth factors such as vascular endothelial growth factor (VEGF) (Tangkeangsirisin, W. & Serrero, G, Carcinogenesis 25:1587-1592 (2004)), and tumorigenesis (He, Z. & Bateman, A., J. Mol. Med. 81:600-612 (2003); Monami, G., et al., Cancer Res. 66:7103-7110 (2006); Serrero, G., Biochem. Biophys. Res. Commun. 308:409-413 (2003); Lu, R. & Serrero, G., Proc. Natl. Acad. Sci. U.S.A. 98:142-147 (2001); Liau, L. M., et al., Cancer Res. 60:1353-1360 (2000)). Both the uncleaved PGRN protein and the derivative GRN peptides have activity in these assays (Zhu, J., et al., Cell 111:867-878 (2002), and in some cases their actions oppose one another.

PGRN is widely expressed in early neural development but becomes restricted in later development to defined neuronal populations, such as cortical neurons, hippocampal pyramidal neurons, and Purkinje cells (Van Damme, P., et al., J. Cell Biol. 181:37-41 (2008); Daniel, R., et al., J. Histochem. Cytochem. 48:999-1009 (2000); Daniel, R., et al., Dev. Dyn. 227:593-599 (2003)). However, not much was known about the role of PGRN in neuronal cells until patients suffering from FTD were shown to carry mutations in the Progranulin gene on chromosome 17 (Baker, M., et al., Nature 442:916-919 (2006); Cruts, M., et al., Nature 442:920-924 (2006)). Subsequently, PGRN has been shown to promote neuronal survival and enhance neurite outgrowth in rat cortical and motor neurons (Van Damme, P., et al., J. Cell Biol. 181:37-41 (2008)). Thus, although PGRN is not a neurotrophin, or a member of the neurotrophin family (see Reichardt, L. F., Phil. Trans. R. Soc. B 361:1545-1564 (2006) for a review discussing the neurotrophin family), it has been referred to as a neurotrophic factor because of its ability to promote neuronal survival. However, until now, the cell surface receptor basis for PGRN and GRN action has not been described for neurons, let alone any cell type.

### Sortilin

Sortilin is variously referred to in the literature as sortilin 1, 100 kDa NT receptor, glycoprotein 95 (GP95), and neurotensin receptor 3 (NT-3 or NTR-3) (Petersen, C. M., et al., J. Biol. Chem. 272:3599-3605 (1997); Mazella, J., et al., J. Biol. Chem. 273:26273-26276 (1998)). Sortilin is an 831 amino acid protein that encodes a type I membrane receptor (Petersen, C. M., et al., J. Biol. Chem. 272:3599-3605 (1997)). Nucleotide and protein sequences of human, rat, and mouse sortilin are provided in SEQ ID NOs:20 and 21, SEQ ID NOs:35 and 36, and SEQ ID NOs:33 and 34, respectively, and the domains of human sortilin are shown in Table 2 (UniProtKB/Swiss-Prot entry Q99523, available from UniProtKB (www.uniprot.org); Petersen, C. M., et al., EMBO J. 18:595-604 (1999); Westergaard, U. B., et al., J. Biol. Chem. 279:50221-50229 (2004)). Sortilin is expressed at high levels in a number of tissues, including the brain, spinal cord, heart and skeletal muscle, thyroid, placenta, and testis (Petersen, C. M., et al., J. Biol. Chem. 272:3599-3605 (1997)).

TABLE 2
Human Sortilin Domains

| Sortilin Domain | Sortilin Sequence (SEQ ID NO: 21) |
|---|---|
| Signal sequence | 1-33 |
| Propeptide | 34-77 |
| Luminal | 78-755 |
| Vps10p | 133-741 |
| | (NCBI Conserved Domain Database |
| | ID: 128865) |
| 10 CC | 612-754 |
| Transmembrane | 756-778 |
| Cytoplasmic | 779-831 |

As one of skill in the art will appreciate, the beginning and ending residues of the domains listed above may vary depending upon the computer modeling program used or the method used for determining the domain.

Sortilin is a member of the VpslOp family of sorting receptors, which also includes SorLA and SorCS1-3 (Bronfman, F. C. & Fainzilber, M., EMBO reports 5:867-871 (2004); Jacobsen, L., et al., J. Biol. Chem. 271:31379-31383 (1996); Westergaard, U. B., et al., J. Biol. Chem. 279:50221-50229 (2004)). The lumenal region of sortilin aligns with each of the two lumenal domains in yeast Vps10p (Vps10p domains) (Petersen, C. M., et al., J. Biol. Chem. 272:3599-3605 (1997)). The hallmark of the Vps10p domain is an amino-terminal propeptide and a carboxy-terminal segment that contains 10 conserved cysteine (10CC) residues (Westergaard, U. B., et al., J. Biol. Chem. 279:50221-50229 (2004)). Other receptors of the VpslOp family share a Vps10p domain, which is situated at the amino-terminus, and contain additional ectodomains (Westergaard, U. B., et al., J. Biol. Chem. 279:50221-50229 (2004)).

The Vps10p family of sorting receptors have diverse functions both within the nervous system and elsewhere. The receptors have been shown to be multifunctional, binding several different ligands, including receptor-associated protein (RAP), neurotensin, lipoprotein lipase, apolipoproteins, and elements of the plasminogen activator system, and engaging in intracellular sorting, endocytosis, and signal transduction (Westergaard, U. B., et al., J. Biol. Chem. 279:50221-50229 (2004)). Sortilin has been shown to mediate rapid endocytosis of lipoprotein lipase, neurotensin, and the proform of nerve growth factor (Nykjær, A., et al., Nature 427:843-848 (2004); Nielsen, M. S., et al., J. Biol. Chem. 274:8832-8836 (1999); Navarro, V., et al., FEBS Lett. 495:100-105 (2001)) and to target proteins for transport from the Golgi to late endosomes (Nielsen, M. S., et al., EMBO J. 20:2180-2190 (2001); Lefrancois, S., et al., EMBO J. 22:6430-6437 (2003)). Further, sortilin has been shown to form a complex with p75^{NTR} on the cell membrane and be essential to pro-nerve growth factor (NGF)-induced neuronal death (Nykjær, A., et al., Nature 427:843-848 (2004)).

Similarly, U.S. Patent Pub. No. US2007/0264195 and International Application No. PCT/DK2007/000567 disclose an interaction between members of the neurotrophin family, which consists of NGF, brain derived neurotrophic factor, neurotrophin-3, and neurotrophin-4/5, or the prodomain form of a neurotrophin (pro-neurotrophin) and members of the Vps10p receptor family. U.S. Patent Pub. No. US2007/0264195 and International Application No. PCT/DK2007/000567 propose methods and compositions for treating various diseases or disorders, including neuronal disorders, by modulating the activity of one of the neurotrophin or pro-neurotrophin family members through the neurotrophin/pro-neurotrophin-Vps10p receptor interaction. However, neither of these published applications disclose or suggest the surprising discovery that sortilin functions as a receptor for PGRN, nor do they disclose or suggest methods for treating diseases or disorders, such as FTD or ALS, by, e.g., modulating the activity of sortilin or the interaction between PGRN and sortilin.

### Antibodies

The methods of the invention may be performed using an antibody or an antigen-binding fragment thereof that specifically binds a sortilin polypeptide. In some embodiments of the methods of the invention, the antibody or an antigen-binding fragment thereof can mimic the binding of PGRN to sortilin.

In some embodiments of the methods of the invention, the anti-sortilin antibody can function as an agonist of sortilin that promotes signaling through the sortilin receptor. In some embodiments of the methods of the invention, the anti-sortilin antibody can function as an antagonist of sortilin that inhibits signaling through the sortilin receptor.

The antibody or antigen-binding fragment for use in the methods of the present invention may be produced in vivo or in vitro. Production of the antibody or antigen-binding fragment is discussed below.

In some embodiments, the anti-sortilin antibody or antigen-binding fragment thereof is murine. In some embodiments, the anti-sortilin antibody or antigen-binding fragment thereof is from mice. In some embodiments, the anti-sortilin antibody or antigen-binding fragment thereof is from rat. In other embodiments, the anti-sortilin antibody or antigen-binding fragment thereof is human. In some embodiments the anti-sortilin antibody or antigen-binding fragment thereof is recombinant, engineered, humanized and/or chimeric.

Exemplary antigen-binding fragments are, Fab, Fab′, F(ab′)2, Fv, Fd, dAb, and fragments containing complementarity determining region (CDR) fragments, single chain antibodies (scFv), chimeric antibodies, diabodies and polypeptides that contain at least a portion of an immunoglobulin that is sufficient to confer specific antigen-binding to the polypeptide (e.g., immunoadhesins).

As used herein, Fd means a fragment that consists of the VH and CH1 domains; Fv means a fragment that consists of the VL and VH domains of a single arm of an antibody; and dAb means a fragment that consists of a VH domain (Ward et al., Nature 341:544-546 (1989)). As used herein, single-chain antibody (scFv) means an antibody in which a VL region and a VH region are paired to form a monovalent molecules via a synthetic linker that enables them to be made as a single protein chain (Bird et al., Science 242:423-426 (1988); Huston et al., Proc. Natl. Acad. Sci. USA 85:5879-5883 (1988)). As used herein, diabody means a bispecific antibody in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger, P., et al., Proc. Natl. Acad. Sci. USA 90:6444-6448 (1993); Poljak, R. J., et al., Structure 2:1121-1123 (1994)). As used herein, immunoadhesin that specifically binds an antigen of interest, means a molecule in which one or more CDRs may be incorporated, either covalently or noncovalently.

In some embodiments, a subunit polypeptide of a sortilin antibody is for use in the methods of the invention, wherein the subunit polypeptide is selected from the group consisting of: (a) a heavy chain or a variable region thereof; and (b) a light chain or a variable region thereof. In some embodiments, the invention provides a nucleic acid encoding the heavy chain or the variable region thereof, or the light chain and the variable region thereof of a subunit polypeptide of a sortilin antibody for use in the methods of the invention. In some embodiments, the invention provides a hypervariable region (CDR) of a sortilin antibody for use in the methods of the invention or a nucleic acid encoding a CDR.

Immunization

Antibodies for use in the methods of the invention can be generated by immunization of a suitable host (e.g., vertebrates, including humans, mice, rats, sheep, goats, pigs, cattle, horses, reptiles, fishes, amphibians, and in eggs of birds, reptiles and fish). Such antibodies may be polyclonal or monoclonal.

In some embodiments, the host is immunized with an immunogenic sortilin. In other embodiments, the host is immunized with sortilin associated with a cell membrane of an intact or disrupted cell and antibodies for use in the methods of the invention are identified by binding to sortilin.

In some embodiments, the sortilin antigen is administered with an adjuvant to stimulate the immune response. Adjuvants often need to be administered in addition to antigen in order to elicit an immune response to the antigen. These adjuvants are usually insoluble or undegradable substances that promote nonspecific inflammation, with recruitment of mononuclear phagocytes at the site of immunization. Examples of adjuvants include, but are not limited to, Freund's adjuvant, RIBI (muramyl dipeptides), ISCOM (immunostimulating complexes) or fragments thereof.

For a review of methods for making antibodies, see, e.g., Harlow and Lane, Antibodies, A Laboratory Manual (1988); Yelton, D. E. et al., Ann. Rev. of Biochem. 50:657-80. (1981); and Ausubel et al., Current Protocols in Molecular Biology (New York: John Wiley & Sons) (1989). Determination of immunoreactivity with an immunogenic sortilin polypeptide may be made by any of several methods well known in the art, including, e.g., immunoblot assay and ELISA.

Production of Antibodies and Antibody Producing Cell Lines

Monoclonal antibodies for use in the methods of the invention can made by standard procedures as described, e.g., in Harlow and Lane, Antibodies, A Laboratory Manual (1988), supra.

Briefly, at an appropriate period of time the animal is sacrificed and lymph node and/or splenic B-cells are immortalized by any one of several techniques that are well-known in the art, including but not limited to transformation, such as with EBV or fusion with an immortalized cell line, such as myeloma cells. Thereafter, the cells are clonally separated and the supernatants of each clone tested for production of an antibody specific for an immunogenic sortilin polypeptide. Methods of selecting, cloning and expanding hybridomas are well known in the art. Similarly, methods for identifying the nucleotide and amino acid sequence of the immunoglobulin genes are known in the art.

Other suitable techniques for producing an antibody for use in the methods of the invention involve in vitro exposure of lymphocytes to sortilin or to an immunogenic polypeptide thereof; or alternatively, selection of libraries of antibodies in phage or similar vectors. See Huse et al., Science 246:1275-81 (1989). Antibodies useful in the methods of the invention may be employed with or without modification.

Antigens and antibodies can be labeled by joining, either covalently or non-covalently, a substance that provides for a detectable signal. Various labels and conjugation techniques are known in the art and can be employed in practicing the invention. Suitable labels include, but are not limited to, radionucleotides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins may be produced (see U.S. Pat. No. 4,816,567).

In some embodiments, an antibody for use in the methods of the invention has multiple binding specificities, such as a bifunctional antibody prepared by any one of a number of techniques known to those of skill in the art including the production of hybrid hybridomas, disulfide exchange, chemical cross-linking, addition of peptide linkers between two monoclonal antibodies, the introduction of two sets of immunoglobulin heavy and light chains into a particular cell line, and so forth (see below for more detailed discussion).

The antibodies of this invention may also be human monoclonal antibodies, for example those produced by immortalized human cells, by SCID-hu mice, or other non-human animals capable of producing “human” antibodies.

Phage Display Libraries

Anti-sortilin antibodies for use in the methods of this invention can be isolated by screening a recombinant combinatorial antibody library. Exemplary combinatorial libraries are for binding to an immunogenic sortilin polypeptide of the invention, such as an scFv phage display library, prepared using VL and VH cDNAs prepared from mRNA derived from an animal immunized with an immunogenic sortilin polypeptide. Methodologies for preparing and screening such libraries are known in the art. There are commercially available methods and materials for generating phage display libraries (e.g., the Pharmacia Recombinant Phage Antibody System, catalog no. 27 9400 01; the Stratagene SURFZAP™ phage display kit, catalog no. 240612; and others from MorphoSys). There are also other methods and reagents that can be used in generating and screening antibody display libraries (see, e.g., U.S. Pat. No. 5,223,409; International Publication No. WO 92/18619; International Publication No. WO 91/17271; International Publication No. WO 92/20791; International Publication No. WO 92/15679; International Publication No. WO 93/01288; International Publication No. WO 92/01047; International Publication No. WO 92/09690; Fuchs et al., Bio/Technology 9:1370-1372 (1991); Hay et al., Hum. Antibod. Hybridomas 3:81-85; (1992) Huse et al., Science 246:1275-1281 (1989); McCafferty et al., Nature 348:552-554 (1990); Griffiths et al., EMBO J. 12:725-734 (1993); Hawkins et al., J. Mol. Biol. 226:889-896 (1992); Clackson et al., Nature 352:624-628 (1991); Gram et al., Proc. Natl. Acad. Sci. USA 89:3576-3580 (1992); Garrad et al., Bio/Technology 9:1373-1377 (1991); Hoogenboom et al., Nucl. Acids Res. 19:4133-4137 (1991); and Barbas et al., Proc. Natl. Acad. Sci. USA 88:7978-7982 (1991).

Following screening and isolation for an anti-sortilin antibody for use in the methods of the invention from a recombinant immunoglobulin display library, the nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention, as described below. To express an antibody isolated by screening a combinatorial library, DNA encoding the antibody heavy chain and light chain or the variable regions thereof is cloned into a recombinant expression vector and introduced into a mammalian host cell, as described above.

Class Switching

Anti-sortilin antibodies for use in the methods of the invention can be of any isotype. An antibody of any desired isotype can be produced by class switching. For class switching, nucleic acids encoding VL or VH, that do not include any nucleotide sequences encoding CL or CH, are isolated using methods well known in the art. The nucleic acids encoding VL or VH are then operatively linked to a nucleotide sequence encoding a CL or CH from a desired class of immunoglobulin molecule. This may be achieved using a vector or nucleic acid that comprises a CL or CH chain, as described above. For example, an anti-sortilin antibody for use in the methods of the invention that was originally IgM may be class switched to an IgG. Further, the class switching may be used to convert one IgG subclass to another, e.g., from IgG1 to IgG2.

Mutated Antibodies

In other embodiments, antibodies or antigen-binding fragments for use in the methods of the invention may be mutated in the variable domains of the heavy and/or light chains to alter a binding property of the antibody. For example, a mutation may be made in one or more of the CDR regions to increase or decrease the Kd of the antibody for sortilin, to increase or decrease K_{off}, or to alter the binding specificity of the antibody. Techniques in site-directed mutagenesis are well known in the art. See, e.g., Sambrook et al., Molecular Cloning—A Laboratory Manual, Cold Spring Harbor Laboratory Press (1989) and Ausubel et al., Current Protocols in Molecular Biology (New York: John Wiley & Sons) (1989). In a preferred embodiment, mutations are made at one or more amino acid residues in a variable region of an anti-sortilin antibody for use in the methods of the invention. In another embodiment, a nucleic acid encoding an antibody heavy chain or light chain variable region is mutated in one or more of the framework regions. A mutation may be made in a framework region or constant domain to increase the half-life. A mutation in a framework region or constant domain also may be made to alter the immunogenicity of the antibody, to provide a site for covalent or non-covalent binding to another molecule, or to alter such properties as complement fixation. Mutations may be made in each of the framework regions, the constant domain and the variable regions in a single mutated antibody. Alternatively, mutations may be made in only one of the framework regions, the variable regions or the constant domain in a single mutated antibody.

Fusion Antibodies and Immunoadhesins

In another embodiment, a fusion antibody or immunoadhesin may be made which comprises all or a portion of an anti-sortilin antibody for use in the methods of the invention linked to another polypeptide. In some embodiments, only the variable region of the anti-sortilin antibody is linked to the polypeptide. In other embodiments, the VH domain of an anti-sortilin antibody is linked to a first polypeptide, while the VL domain of the antibody is linked to a second polypeptide that associates with the first polypeptide in a manner that permits the VH and VL domains to interact with one another to form an antibody binding site. In other embodiments, the VH domain is separated from the VL domain by a linker that permits the VH and VL domains to interact with one another (see below under Single Chain Antibodies). The VH-linker-VL antibody is then linked to a polypeptide of interest. The fusion antibody is useful to directing a polypeptide to a cell or tissue that expresses PGRN. The polypeptide of interest may be a therapeutic agent, such as a toxin, or may be a diagnostic agent, such as an enzyme that may be easily visualized, such as horseradish peroxidase. In addition, fusion antibodies can be created in which two (or more) single-chain antibodies are linked to one another. This is useful if one wants to create a divalent or polyvalent antibody on a single polypeptide chain, or if one wants to create a bispecific antibody.

Single Chain Antibodies

The present invention includes a single chain antibody (scFv) that binds sortilin. To produce the ScFv, VH and VL encoding DNA is operatively linked to DNA encoding a flexible linker, e.g., encoding the amino acid sequence (Gly₄Ser)₃ (SEQ ID NO:1), such that the VH and VL sequences can be expressed as a contiguous single chain protein, with the VL and VH regions joined by the flexible linker (see, e.g., Bird et al., Science 242:423-426 (1988); Huston et al., Proc. Natl. Acad. Sci. USA 85:5879-5883 (1988); McCafferty et al., Nature 348:552-554 (1990)). The single chain antibody may be monovalent, if only a single VH and VL are used, bivalent, if two VH and VL are used, or polyvalent, if more than two VH and VL are used.

Chimeric Antibodies

The present invention further includes a bispecific antibody or antigen-binding fragment thereof in which one specificity is for sortilin. In one embodiment, a chimeric antibody can be generated that specifically binds to sortilin through one binding domain and to a second molecule through a second binding domain. The chimeric antibody can be produced through recombinant molecular biological techniques, or may be physically conjugated together. In addition, a single chain antibody containing more than one VH and VL may be generated that binds specifically to sortilin and to another molecule that is associated with FTD or ALS. Such bispecific antibodies can be generated using techniques that are well known for example, Fanger et al., Immunol. Methods 4 72-81 (1994) and Wright and Harris, supra.

In some embodiments, the chimeric antibodies are prepared using one or more of the variable regions from an antibody for use in the methods of the invention. In another embodiment, the chimeric antibody is prepared using one or more CDR regions from said antibody.

Derivatized and Labeled Antibodies

An antibody or an antigen-binding fragment of the invention can be derivatized or linked to another molecule (e.g., another peptide or protein). In general, the antibody or antigen-binding fragment is derivatized such that binding to the antigen is not affected adversely by the derivatization or labeling. For example, an antibody or antibody portion for use in the methods of the invention can be functionally linked (by chemical coupling, genetic fusion, noncovalent association or otherwise) to one or more other molecular entities, such as another antibody (e.g., a bispecific antibody or a diabody), a detection agent, a cytotoxic agent, a pharmaceutical agent, and/or a protein or peptide that can mediate association of the antibody or antigen-binding fragment with another molecule (such as a streptavidin core region or a polyhistidine tag).

In some embodiments, a derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl N-hydroxysuccinimide ester) or homobifunctional (e.g., disuccinimidyl suberate). Such linkers are available from Pierce Chemical Company, Rockford, Ill.

In some embodiments, the derivatized antibody is a labeled antibody. Exemplary, detection agents with which an antibody or antibody portion for use in the methods of the invention may be derivatized are fluorescent compounds, including fluorescein, fluorescein isothiocyanate, rhodamine, 5-dimethylamine-1-napthalenesulfonyl chloride, phycoerythrin, lanthanide phosphors and the like. An antibody also may be labeled with enzymes that are useful for detection, such as horseradish peroxidase, β-galactosidase, luciferase, alkaline phosphatase, glucose oxidase and the like. In embodiments that are labeled with a detectable enzyme, the antibody is detected by adding additional reagents that the enzyme uses to produce a detectable reaction product. For example, horseradish peroxidase with hydrogen peroxide and diaminobenzidine. An antibody also may be labeled with biotin, and detected through indirect measurement of avidin or streptavidin binding. An antibody may also be labeled with a predetermined polypeptide epitope recognized by a secondary reporter (e.g., leucine zipper pair sequences, binding sites for secondary antibodies, metal binding domains, epitope tags).

An anti-sortilin antibody or an antigen-fragment thereof also may be labeled with a radiolabeled amino acid. The radiolabel may be used for both diagnostic and therapeutic purposes. The radiolabeled anti-sortilin antibody may be used diagnostically, for example, for determining sortilin levels in a subject. Further, the radiolabeled anti-sortilin antibody may be used therapeutically for treating spinal cord injury. Examples of labels for polypeptides include, but are not limited to, the following radioisotopes or radionucleotides—³H, ¹⁴C, ¹⁵N, ³⁵S, ⁹⁰Y, ⁹⁹Tc, ¹¹¹In, ¹²⁵I, ¹³¹I.

An anti-sortilin antibody or an antigen-fragment thereof may also be derivatized with a chemical group such as polyethylene glycol (PEG), a methyl or ethyl group, or a carbohydrate group. These groups may be useful to improve the biological characteristics of the antibody, e.g., to increase serum half-life or to increase tissue binding.

Characterization of Anti-Sortilin Antibodies

Class and Subclass of Anti-Sortilin Antibodies

The class and subclass of anti-sortilin antibodies may be determined by any method known in the art. In general, the class and subclass of an antibody may be determined using antibodies that are specific for a particular class and subclass of antibody. Such antibodies are available commercially. The class and subclass can be determined by ELISA, Western Blot, as well as other techniques. Alternatively, the class and subclass may be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies.

Binding Affinity of Anti-Sortilin Antibody to Sortilin

The binding affinity and dissociation rate of an anti-sortilin antibody for use in the methods of the invention to a sortilin polypeptide may be determined by any method known in the art. For example, the binding affinity can be measured by competitive ELISAs, RIAs, BIACORE™, or KINEXA™ technology. The dissociation rate also can be measured by BIACORE™ or KINEXA™ technology. The binding affinity and dissociation rate are measured by surface plasmon resonance using, e.g., a BIACORE™.

Modulation of Sortilin Activity by Anti-Sortilin Antibody

In some embodiments, an anti-sortilin antibody or an antigen-binding fragment for use in the methods of the invention thereof modulates the binding of PGRN to sortilin. In some embodiments, the modulation is enhancement of the binding of PGRN to sortilin. In some embodiments, the modulation is inhibition of the binding of PGRN to sortilin. The IC₅₀ of such inhibition can be measured by any method known in the art, e.g., by ELISA, RIA, or Functional Antagonism. In some embodiments, the IC₅₀ is between 0.1 and 500 nM. In some embodiments, the IC₅₀ is between 10 and 400 nM. In yet other embodiments, the antibody or portion thereof has an IC₅₀ of between 60 nM and 400 nM.

In some embodiments, the methods of the present invention include sortilin-specific antibodies or antigen-binding fragments, variants, or derivatives which are agonists of sortilin activity. In some embodiments, the methods of the present invention also include sortilin-specific antibodies or antigen-binding fragments, variants, or derivatives which are antagonists of sortilin activity.

In other embodiments, the methods of the present invention include an antibody, or antigen-binding fragment, variant, or derivative thereof which specifically or preferentially binds to at least one epitope of sortilin, where the epitope comprises, consists essentially of, or consists of at least about four to five amino acids of SEQ ID NOs:21, 34, or 36, at least seven, at least nine, or between at least about 15 to about 30 amino acids of SEQ ID NOs:21, 34, or 36. The amino acids of a given epitope of SEQ ID NOs:21, 34, or 36 as described may be, but need not be contiguous or linear. In certain embodiments, the at least one epitope of sortilin comprises, consists essentially of, or consists of a non-linear epitope formed by the extracellular domain of sortilin as expressed on the surface of a cell or as a soluble ectodomain fragment, e.g., fused to an IgG Fc region. Thus, in certain embodiments the at least one epitope of sortilin comprises, consists essentially of, or consists of at least 4, at least 5, at least 6, at least 7, at least 8, at least 9, at least 10, at least 15, at least 20, at least 25, between about 15 to about 30, or at least 10, 15, 20, 25, 30, 35, 40, 45, 50, 55, 60, 65, 70, 75, 80, 85, 90, 95, or 100 contiguous or non-contiguous amino acids of SEQ ID NOs:21, 34, or 36, where non-contiguous amino acids form an epitope through protein folding.

In other embodiments, the methods of the present invention include an antibody, or antigen-binding fragment, variant, or derivative thereof which specifically or preferentially binds to at least one epitope of sortilin, where the epitope comprises, consists essentially of, or consists of, in addition to one, two, three, four, five, six or more contiguous or non-contiguous amino acids of SEQ ID NOs:21, 34, or 36 as described above, and an additional moiety which modifies the protein, e.g., a carbohydrate moiety may be included such that the sortilin antibody binds with higher affinity to modified target protein than it does to an unmodified version of the protein. Alternatively, the sortilin antibody does not bind the unmodified version of the target protein at all.

In certain embodiments, an antibody, or antigen-binding fragment, variant, or derivative thereof for use in the methods of the invention binds specifically to at least one epitope of sortilin or a fragment or variant described above, i.e., binds to such an epitope more readily than it would bind to an unrelated, or random epitope; binds preferentially to at least one epitope of sortilin or fragment or variant described above, i.e., binds to such an epitope more readily than it would bind to a related, similar, homologous, or analogous epitope; competitively inhibits binding of a reference antibody which itself binds specifically or preferentially to a certain epitope of sortilin or fragment or variant described above; or binds to at least one epitope of sortilin or fragment or variant described above with an affinity characterized by a dissociation constant Kd of less than about 5×10⁻² M, about 10⁻² M, about 5×10⁻³ M, about 10⁻³ M, about 5×10⁻⁴ M, about 10⁻⁴ M, about 5×10⁻⁵ M, about 10⁻⁵ M, about 5×10⁻⁶ M, about 10⁻⁶ M, about 5×10⁻⁷ M, about 10⁻⁷ M, about 5×10⁻⁸ M, about 10⁻⁸ M, about 5×10⁻⁹ M,about 10⁻⁹ M, about 5×10⁻¹⁰ M, about 10⁻¹⁰ M, about 5×10⁻¹¹ M, about 10⁻¹¹ M, about 5×10⁻¹² M, about 10⁻¹² M, about 5×10⁻¹³ M, about 10⁻¹³ M, about 5×10⁻¹⁴ M, about 10⁻¹⁴ M, about 5×10⁻¹⁵ M, or about 10⁻¹⁵ M. In a particular aspect, the antibody or fragment thereof preferentially binds to a human sortilin polypeptide or fragment thereof, relative to a murine sortilin polypeptide or fragment thereof.

As used in the context of antibody binding dissociation constants, the term “about” allows for the degree of variation inherent in the methods utilized for measuring antibody affinity. For example, depending on the level of precision of the instrumentation used, standard error based on the number of samples measured, and rounding error, the term “about 10⁻² M” might include, for example, from 0.05 M to 0.005 M.

In specific embodiments, an antibody, or antigen-binding fragment, variant, or derivative thereof for use in the methods of the invention binds sortilin polypeptides or fragments or variants thereof with an off rate (k(off)) of less than or equal to 5×10⁻² sec-1, 10⁻² sec-1, 5×10⁻³ sec-1 or 10⁻³ sec-1. Alternatively, an antibody, or antigen-binding fragment, variant, or derivative thereof of the invention binds sortilin polypeptides or fragments or variants thereof with an off rate (k(off)) of less than or equal to 5×10⁻⁴ sec-1, 10⁻⁴ sec-1, 5×10⁻⁵ sec-1, or 10⁻⁵ sec-1 5×10⁻⁶ sec-1, 10⁻⁶ sec-1, 5×10⁻⁷ sec-1 or 10⁻⁷ sec-1.

In other embodiments, an antibody, or antigen-binding fragment, variant, or derivative thereof for use in the methods of the invention binds sortilin polypeptides or fragments or variants thereof with an on rate (k(on)) of greater than or equal to 10³ M-1 sec-1, 5×10³ M-1 sec-1, 10⁴ M-1 sec-1, or 5×10⁴ M-1 sec-1. Alternatively, an antibody, or antigen-binding fragment, variant, or derivative thereof for use in the methods of the invention binds sortilin polypeptides or fragments or variants thereof with an on rate (k(on)) greater than or equal to 10⁵ M-1 sec-1, 5×10⁵ M-1 sec-1, 10⁶ M-1 sec-1, or 5×10⁶ M-1 sec-1 or 10⁷ M-1 sec-1.

Unless it is specifically noted, as used herein a “fragment thereof” in reference to an antibody refers to an immunospecific fragment, i.e., an antigen-specific fragment. In one embodiment, an antibody of the invention is a bispecific binding molecule, binding polypeptide, or antibody, e.g., a bispecific antibody, minibody, domain deleted antibody, or fusion protein having binding specificity for more than one epitope, e.g., more than one antigen or more than one epitope on the same antigen. In one embodiment, a bispecific antibody has at least one binding domain specific for at least one epitope on sortilin. A bispecific antibody may be a tetravalent antibody that has two target binding domains specific for an epitope of sortilin and two target binding domains specific for a second target. Thus, a tetravalent bispecific antibody may be bivalent for each specificity.

In certain embodiments of the methods of the present invention, the methods comprise administration of an anti-sortilin antibody, or immunospecific fragment thereof, in which at least a fraction of one or more of the constant region domains has been deleted or otherwise altered so as to provide desired biochemical characteristics such as reduced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies for use in the treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted.

In certain sortilin antibodies or immunospecific fragments thereof for use in the therapeutic methods described herein, the Fc portion may be mutated to alter, e.g., increase, decrease, or modulate effector function using techniques known in the art. For example, the deletion or inactivation (through point mutations or other means) of a constant region domain may reduce or alter Fc receptor binding of the circulating modified antibody thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or antibody flexibility. The resulting physiological profile, bioavailability and other biochemical effects of the modifications, such as tumor localization, biodistribution and serum half-life, may easily be measured and quantified using well know immunological techniques without undue experimentation.

Modified forms of antibodies or immunospecific fragments thereof for use in the diagnostic and therapeutic methods disclosed herein can be made from whole precursor or parent antibodies using techniques known in the art. Exemplary techniques are discussed in more detail herein.

In certain embodiments both the variable and constant regions of sortilin antibodies or immunospecific fragments thereof for use in the treatment methods disclosed herein are fully human. Fully human antibodies can be made using techniques that are known in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140. Other techniques are known in the art. Fully human antibodies can likewise be produced by various display technologies, e.g., phage display or other viral display systems, as described in more detail elsewhere herein.

Anti-sortilin antibodies or immunospecific fragments thereof for use in the treatment methods disclosed herein include derivatives that are modified, e.g., by the covalent attachment of any type of molecule to the antibody such that covalent attachment does not prevent the antibody from specifically binding to its cognate epitope. For example, but not by way of limitation, the antibody derivatives include antibodies that have been modified, e.g., by glycosylation, acetylation, pegylation, phosphorylation, amidation, or derivatization by known protecting/blocking groups, proteolytic cleavage, linkage to a cellular ligand or other protein. Any of numerous chemical modifications may be carried out by known techniques, including, but not limited to, specific chemical cleavage, acetylation, formylation, metabolic synthesis of tunicamycin. Additionally, the derivative may contain one or more non-classical amino acids.

In preferred embodiments, an anti-sortilin antibody or immunospecific fragment thereof for use in the methods disclosed herein will not elicit a deleterious immune response in the mammal to be treated, e.g., in a human. In one embodiment, the anti-sortilin antibodies or immunospecific fragments thereof for use in the methods disclosed herein may be modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. USA 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089; 5,693,761; 5,693,762; and 6,190,370, all of which are hereby incorporated by reference in their entirety.

De-immunization can also be used to decrease the immunogenicity of an antibody. As used herein, the term “de-immunization” includes alteration of an antibody to modify T cell epitopes (see, e.g., WO9852976A1, WO0034317A2). For example, VH and VL sequences from the starting antibody are analyzed and a human T cell epitope “map” from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are designed comprising combinations of amino acid substitutions and these sequences are subsequently incorporated into a range of binding polypeptides, e.g., anti-sortilin antibodies or immunospecific fragments thereof for use in the methods disclosed herein, which are then tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

Anti-sortilin antibodies or fragments thereof for use in the methods of the present invention may be generated by any suitable method known in the art. Polyclonal antibodies can be produced by various procedures well known in the art. For example, a sortilin immunospecific fragment can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, Cold Spring Harbor Laboratory Press, 2nd ed. (1988); Hammerling et al., in: Monoclonal Antibodies and T-Cell Hybridomas Elsevier, N.Y., 563-681 (1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Thus, the term “monoclonal antibody” is not limited to antibodies produced through hybridoma technology. Monoclonal antibodies can be prepared using a wide variety of techniques known in the art including the use of hybridoma and recombinant and phage display technology.

Using art recognized protocols, in one example, antibodies are raised in mammals by multiple subcutaneous or intraperitoneal injections of the relevant antigen (e.g., purified sortilin antigens or cells or cellular extracts comprising such antigens) and an adjuvant. This immunization typically elicits an immune response that comprises production of antigen-reactive antibodies from activated splenocytes or lymphocytes. While the resulting antibodies may be harvested from the serum of the animal to provide polyclonal preparations, it is often desirable to isolate individual lymphocytes from the spleen, lymph nodes or peripheral blood to provide homogenous preparations of monoclonal antibodies. Preferably, the lymphocytes are obtained from the spleen.

In this well known process (Kohler et al., Nature 256:495-497 (1975)) the relatively short-lived, or mortal, lymphocytes from a mammal which has been injected with antigen are fused with an immortal tumor cell line (e.g., a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal.”

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by in vitro assays such as immunoprecipitation, radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, Academic Press, pp 59-103 (1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

Antibody fragments that recognize specific epitopes may be generated by known techniques. For example, Fab and F(ab′)2 fragments may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain.

Those skilled in the art will also appreciate that DNA encoding antibodies or antibody fragments (e.g., antigen binding sites) may also be derived from antibody phage libraries. In a particular embodiment, such phage can be utilized to display antigen-binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Exemplary methods are set forth, for example, in EP 368 684 B1; U.S. Pat. No. 5,969,108; Hoogenboom, H. R. and Chames, Immunol. Today 21:371-378 (2000); Nagy et al., Nature Med. 8:801-807 (2002); Huie et al., Proc. Natl. Acad. Sci. USA 98:2682-2687 (2001); Lui et al., J. Mol. Biol. 315:1063-1072 (2002), each of which is incorporated herein by reference. Several publications (e.g., Marks et al., Bio/Technology 10:779-783 (1992)) have described the production of high affinity human antibodies by chain shuffling, as well as combinatorial infection and in vivo recombination as a strategy for constructing large phage libraries. In another embodiment, Ribosomal display can be used to replace bacteriophage as the display platform (see, e.g., Hanes et al., Nature Biotechnol. 18:1287-1292 (2000); Wilson et al., Proc. Natl. Acad. Sci. USA 98:3750-3755 (2001); Irving et al., J. Immunol. Methods 248:31-45 (2001)). In yet another embodiment, cell surface libraries can be screened for antibodies (Boder et al., Proc. Natl. Acad. Sci. USA 97:10701-10705 (2000); Daugherty et al., J. Immunol. Methods 243:211-227 (2000)). Such procedures provide alternatives to traditional hybridoma techniques for the isolation and subsequent cloning of monoclonal antibodies.

In phage display methods, functional antibody domains are displayed on the surface of phage particles which carry the polynucleotide sequences encoding them. In particular, DNA sequences encoding VH and VL regions are amplified from animal cDNA libraries (e.g., human or murine cDNA libraries of lymphoid tissues) or synthetic cDNA libraries. In certain embodiments, the DNA encoding the VH and VL regions are joined together by an scFv linker by PCR and cloned into a phagemid vector (e.g., pCANTAB 6 or pComb 3 HSS). The vector is electroporated in E. coli and the E. coli is infected with helper phage. Phage used in these methods are typically filamentous phage including fd and M13 and the VH or VL regions are usually recombinantly fused to either the phage gene III or gene VIII. Phage expressing an antigen binding domain that binds to an antigen of interest (i.e., a sortilin polypeptide or a fragment thereof) can be selected or identified with antigen, e.g., using labeled antigen or antigen bound or captured to a solid surface or bead.

Additional examples of phage display methods that can be used to make the antibodies include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Adv. in Immunol. 57:191-280 (1994); PCT Application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria. For example, techniques to recombinantly produce Fab, Fab′ and F(ab′)2 fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., Proc. Natl. Acad. Sci. USA 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are known in the art. See, e.g., Morrison, Science 229:1202 (1985); Oi et al., BioTechniques 4:214 (1986); Gillies et al., J. Immunol. Methods 125:191-202 (1989); U.S. Pat. Nos. 5,807,715; 4,816,567; and 4,816,397, which are incorporated herein by reference in their entireties. Humanized antibodies are antibody molecules from non-human species antibody that binds the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; Riechmann et al., Nature 332:323-327 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., Proc. Natl. Acad. Sci. USA 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a desired target polypeptide. Monoclonal antibodies directed against the antigen can be obtained from the immunized, transgenic mice using conventional hybridoma technology. The human immunoglobulin transgenes harbored by the transgenic mice rearrange during B-cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/Technology 12:899-903 (1988)). See also, U.S. Pat. No. 5,565,332.

In another embodiment, DNA encoding desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be synthetic as described herein) may be used to clone constant and variable region sequences for the manufacture antibodies as described in Newman et al., U.S. Pat. No. 5,658,570, filed Jan. 25, 1995, which is incorporated by reference herein. Essentially, this entails extraction of RNA from the selected cells, conversion to cDNA, and amplification by PCR using Ig specific primers. Suitable primers for this purpose are also described in U.S. Pat. No. 5,658,570. As will be discussed in more detail below, transformed cells expressing the desired antibody may be grown up in relatively large quantities to provide clinical and commercial supplies of the immunoglobulin.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds to at least one epitope of a desired polypeptide, e.g., sortilin. Preferably, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. USA 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As used herein, a chimeric antibody is a molecule in which different portions are derived from different animal species, such as those having a variable region derived from a murine monoclonal antibody and a human immunoglobulin constant region, e.g., humanized antibodies.

Alternatively, techniques described for the production of single chain antibodies (U.S. Pat. No. 4,694,778; Bird, Science 242:423-442 (1988); Huston et al., Proc. Natl. Acad. Sci. USA 85:5879-5883 (1988); and Ward et al., Nature 334:544-554 (1989)) can be adapted to produce single chain antibodies. Single chain antibodies are formed by linking the heavy and light chain fragments of the Fv region via an amino acid bridge, resulting in a single chain antibody. Techniques for the assembly of functional Fv fragments in E coli may also be used (Skerra et al., Science 242:1038-1041 (1988)).

Anti-sortilin antibodies may also be human or substantially human antibodies generated in transgenic animals (e.g., mice) that are incapable of endogenous immunoglobulin production (see, e.g., U.S. Pat. Nos. 6,075,181; 5,939,598; 5,591,669; and 5,589,369, each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524, which is incorporated herein by reference. It will be appreciated that the genetic material associated with these human antibodies may also be isolated and manipulated as described herein.

Yet another highly efficient means for generating recombinant antibodies is disclosed by Newman, Biotechnology 10:1455-1460 (1992). Specifically, this technique results in the generation of primatized antibodies that contain monkey variable domains and human constant sequences. This reference is incorporated by reference in its entirety herein.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

Alternatively, antibody-producing cell lines may be selected and cultured using techniques well known to the skilled artisan. Such techniques are described in a variety of laboratory manuals and primary publications. In this respect, techniques suitable for use in the invention as described below are described in Current Protocols in Immunology, Coligan et al., Eds., Green Publishing Associates and Wiley-Interscience, John Wiley and Sons, New York (1991) which is herein incorporated by reference in its entirety, including supplements.

Antibodies for use in the methods disclosed herein can be produced by any method known in the art for the synthesis of antibodies, in particular, by chemical synthesis or preferably, by recombinant expression techniques as described herein.

It will further be appreciated that the scope of the methods of this invention further encompasses all alleles, variants and mutations of antigen binding DNA sequences.

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligo dT cellulose. Suitable techniques are familiar in the art.

In one embodiment, cDNAs that encode the light and the heavy chains of the antibody may be made, either simultaneously or separately, using reverse transcriptase and DNA polymerase in accordance with well known methods. PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells using techniques known in the art, restriction mapped and sequenced in accordance with standard, well known techniques set forth in detail, e.g., in the foregoing references relating to recombinant DNA techniques. Of course, the DNA may be synthetic according to the present invention at any point during the isolation process or subsequent analysis.

Recombinant expression of an antibody, or fragment, derivative or analog thereof, e.g., a heavy or light chain of an antibody which is an anti-sortilin antibody, requires construction of an expression vector containing a polynucleotide that encodes the antibody. Once a polynucleotide encoding an antibody molecule or a heavy or light chain of an antibody, or portion thereof (preferably containing the heavy or light chain variable domain), has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody for use in the methods described herein. Thus, the invention includes host cells containing a polynucleotide encoding an antibody for use in the methods of the invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express antibody molecules for use in the methods described herein. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as E. coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101-105 (1986); Cockett et al., Bio/Technology 8:662-667 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791-1794 (1983)), in which the antibody coding sequence may be ligated individually into the vector in frame with the lacZ coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is typically used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements or transcription terminators. (Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which stably express the antibody molecule.

A number of selection systems may be used, including but not limited to the herpes simplex virus thymidine kinase (Wigler et al., Cell 11:223-232 (1977)), hypoxanthine-guanine phosphoribosyltransferase (Szybalska & Szybalski, Proc. Natl. Acad. Sci. USA 48:2026-2034 (1962)), and adenine phosphoribosyltransferase (Lowy et al., Cell 22:817-823 (1980)) genes can be employed in tk-, hgprt- or aprt-cells, respectively. Also, antimetabolite resistance can be used as the basis of selection for the following genes: dhfr, which confers resistance to methotrexate (Wigler et al., Proc. Natl. Acad. Sci. USA 77:357-361 (1980); O'Hare et al., Proc. Natl. Acad. Sci. USA 78:1527-1531 (1981)); gpt, which confers resistance to mycophenolic acid (Mulligan & Berg, Proc. Natl. Acad. Sci. USA 78:2072-2076 (1981)); neo, which confers resistance to the aminoglycoside G-418 (Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993)); and hygro, which confers resistance to hygromycin (Santerre et al., Gene 30:147-156 (1984). Methods commonly known in the art of recombinant DNA technology which can be used are described in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Academic Press, New York, Vol. 3. (1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257-266 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes both heavy and light chain polypeptides. In such situations, the light chain is advantageously placed before the heavy chain to avoid an excess of toxic free heavy chain (Proudfoot, Nature 322:52 (1986); Kohler, Proc. Natl. Acad. Sci. USA 77:2197-2199 (1980)). The coding sequences for the heavy and light chains may comprise cDNA or genomic DNA.

Once an antibody molecule of the invention has been recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Alternatively, a preferred method for increasing the affinity of antibodies of the invention is disclosed in US 2002/0123057 A1.

In one embodiment, a binding molecule or antigen binding molecule for use in the methods of the invention comprises a synthetic constant region wherein one or more domains are partially or entirely deleted (“domain-deleted antibodies”). In certain embodiments compatible modified antibodies will comprise domain deleted constructs or variants wherein the entire CH2 domain has been removed (ACH2 constructs). For other embodiments a short connecting peptide may be substituted for the deleted domain to provide flexibility and freedom of movement for the variable region. Those skilled in the art will appreciate that such constructs are particularly preferred due to the regulatory properties of the CH2 domain on the catabolic rate of the antibody.

In certain embodiments, modified antibodies for use in the methods disclosed herein are minibodies. Minibodies can be made using methods described in the art (see, e.g., U.S. Pat. No. 5,837,821 or WO 94/09817A1).

The present invention also provides the use of antibodies that comprise, consist essentially of, or consist of, variants (including derivatives) of antibody molecules (e.g., the VH regions and/or VL regions) described herein, which antibodies or fragments thereof immunospecifically bind to a sortilin polypeptide. Standard techniques known to those of skill in the art can be used to introduce mutations in the nucleotide sequence encoding a binding molecule, including, but not limited to, site-directed mutagenesis and PCR-mediated mutagenesis which result in amino acid substitutions. Preferably, the variants (including derivatives) encode less than 50 amino acid substitutions, less than 40 amino acid substitutions, less than 30 amino acid substitutions, less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the reference VH region, VHCDR1, VHCDR2, VHCDR3, VL region, VLCDR1, VLCDR2, or VLCDR3. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity.

For example, it is possible to introduce mutations only in framework regions or only in CDR regions of an antibody molecule. Introduced mutations may be silent or neutral missense mutations, i.e., have no, or little, effect on an antibody's ability to bind antigen. These types of mutations may be useful to optimize codon usage, or improve a hybridoma's antibody production. Alternatively, non-neutral missense mutations may alter an antibody's ability to bind antigen. The location of most silent and neutral missense mutations is likely to be in the framework regions, while the location of most non-neutral missense mutations is likely to be in CDR, though this is not an absolute requirement. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein may routinely be expressed and the functional and/or biological activity of the encoded protein can be determined using techniques described herein or by routinely modifying techniques known in the art.

In sum, one of skill in the art, provided with the teachings of this invention, has available a variety of methods which may be used to alter the biological properties of the antibodies of this invention including methods which would increase or decrease the stability or half-life, immunogenicity, toxicity, affinity or yield of a given antibody molecule, or to alter it in any other way that may render it more suitable for a particular application.

### PGRN or Sortilin Polypeptides

Full-length PGRN consists of a signal sequence, 7.5 repeats of smaller granulin motifs referred to as granulin A-G and paragranulin, and a C-terminus (see Table 1). Some embodiments of the invention provide a full-length PGRN for use in the methods of the invention. In some embodiments, smaller granulin motifs are provided for use in the methods of the invention, such as granulin A-G or paragranulin. In some embodiments, a C-terminal polypeptide is provided for use in the methods of the invention that comprises the carboxy-terminal 17 amino acids.

Full-length sortilin consists of a signal sequence, a propeptide, a luminal domain, which comprises a Vps10p domain, a transmembrane domain, and a cytoplasmic domain (see Table 2). The Vps10p domain comprises a 10CC domain. Some embodiments of the invention provide a full-length sortilin for use in the methods of the invention. In some embodiments, a soluble sortilin polypeptide is provided for use in the methods of the invention that lacks a transmembrane domain. In some embodiments, a soluble sortilin polypeptide is provided for use in the methods of the invention that comprises a luminal domain and lacks a transmembrane domain. In some embodiments, a soluble sortilin polypeptide is provided for use in the methods of the invention that comprises a Vps10p domain.

In some embodiments of the invention, the PGRN polypeptides are used to modulate the interaction between PGRN and sortilin. The use of PGRN polypeptides can modulate the interaction between PGRN and sortilin by increasing the activity of the interaction or by decreasing the activity of the interaction. In some embodiments of the invention, the PGRN polypeptides are used to modulate the activity of sortilin. The use of PGRN polypeptides can modulate the activity of sortilin by increasing the activity of sortilin or by decreasing the activity of sortilin.

In some embodiments of the invention, the sortilin polypeptides are used to modulate the interaction between PGRN and sortilin. The use of sortilin polypeptides can modulate the interaction between PGRN and sortilin by increasing the activity of the interaction or by decreasing the activity of the interaction. In some embodiments of the invention, the sortilin polypeptides are used to modulate the activity of sortilin. The use of sortilin polypeptides can modulate the activity of sortilin by increasing the activity of sortilin or by decreasing the activity of sortilin. In some embodiments of the invention, the sortilin polypeptides are used to modulate the activity of PGRN. The use of sortilin polypeptides can modulate the activity of PGRN by increasing the activity of PGRN or by decreasing the activity of PGRN.

In another embodiment, the methods of the present invention provide an isolated polypeptide fragment of 200 residues or less, 150 residues or less, 100 residues or less, 90 residues or less, 80 residues or less, 70 residues or less, 60 residues or less, 50 residues or less, 40 residues or less, 30 residues or less, 20 residues or less, or 15 residues or less, comprising an amino acid sequence identical to a reference amino acid sequence, except for up to one, two, three, four, five, ten, fifteen, or twenty individual amino acid substitutions, depending on the length of the fragment, for use in the methods of the invention.

In some embodiments of the invention, the progranulin or sortilin polypeptides for use in the methods of the invention are 70%, 75%, 80%, 85%, 90%, 95%, or 100% identical to a reference amino acid sequence selected from the group consisting of: (a) SEQ ID NO: 10, (b) SEQ ID NO: 11, (c) SEQ ID NO:12, (d) SEQ ID NO:13, (e) SEQ ID NO:14, (f) SEQ ID NO:SEQ ID NO:15, (g) SEQ ID NO:16, (h) SEQ ID NO:17, (i) SEQ ID NO:18, (j) SEQ ID NO:19, (k) SEQ ID NO:21, (l) SEQ ID NO:30, (m) SEQ ID NO:32, (n) SEQ ID NO:34, (o) SEQ ID NO:36, (p) amino acids 577-593 of SEQ ID NO:30, (q) amino acids 584-602 of SEQ ID NO:32 (r) variants or derivatives of any of said reference amino acid sequences; and (s) a combination of one or more of said reference amino acid sequences or variants or derivatives thereof.

By “a reference amino acid sequence” is meant the specified sequence, e.g., a PGRN or a sortilin amino acid sequence, without the introduction of any amino acid substitutions. As one of ordinary skill in the art would understand, if there are no substitutions, an isolated polypeptide for use in the methods of the invention comprises an amino acid sequence which is identical to the reference amino acid sequence.

Amino acid substitutions for the amino acids of the polypeptides for use in the methods of the invention and the reference amino acid sequence can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Typical amino acids to substitute for cysteines in the reference amino acid include alanine, serine, threonine, in particular, alanine. Making such substitutions through engineering of a polynucleotide encoding the polypeptide fragment is well within the routine expertise of one of ordinary skill in the art. Which different amino acid is used depends on a number of criteria, for example, the effect of the substitution on the conformation of the polypeptide fragment, the charge of the polypeptide fragment, or the hydrophilicity of the polypeptide fragment.

In the methods of the present invention, a polypeptide for use in the methods of the invention can be composed of amino acids joined to each other by peptide bonds or modified peptide bonds, i.e., peptide isosteres, and may contain amino acids other than the 20 gene-encoded amino acids (e.g., non-naturally occurring amino acids). The polypeptides for use in the methods of the present invention may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from natural processes or may be made by synthetic methods. Modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, 2nd Ed., T. E. Creighton, W.H. Freeman and Company, New York (1993); Posttranslational Covalent Modification of Proteins, B.C. Johnson, Ed., Academic Press, New York, pgs. 1-12 (1983); Seifter et al., Meth. Enzymol. 182:626-646 (1990); Rattan et al., Ann. NY Acad. Sci. 663:48-62 (1992).).

Polypeptides for use in the methods of the invention may be cyclic. Cyclization of the polypeptides reduces the conformational freedom of linear peptides and results in a more structurally constrained molecule. Many methods of peptide cyclization are known in the art. For example, “backbone to backbone” cyclization by the formation of an amide bond between the N-terminal and the C-terminal amino acid residues of the peptide. The “backbone to backbone” cyclization method includes the formation of disulfide bridges between two α-thio amino acid residues (e.g., cysteine, homocysteine). Certain peptides of the present invention include modifications on the N- and C-terminus of the peptide to form a cyclic polypeptide. Such modifications include, but are not limited, to cysteine residues, acetylated cysteine residues, cysteine residues with a NH₂ moiety and biotin. Other methods of peptide cyclization are described in Li & Roller, Curr. Top. Med. Chem. 3:325-341 (2002) and U.S Patent Publication No. U.S. 2005-0260626 A1, which are incorporated by reference herein in their entirety.

Fusion Proteins and Conjugated Polypeptides

Some embodiments of the invention involve the use of a polypeptide in the methods of the invention that is not the full-length PGRN or sortilin protein, fused to a heterologous polypeptide to form a fusion protein. Such fusion proteins can be used to accomplish various objectives, e.g., increased serum half-life, improved bioavailability, in vivo targeting to a specific organ or tissue type, improved recombinant expression efficiency, improved host cell secretion, ease of purification, and higher avidity. Depending on the objective(s) to be achieved, the heterologous polypeptide can be inert or biologically active. Also, it can be chosen to be stably fused to the polypeptide for use in the methods of the invention or to be cleavable, in vitro or in vivo. Heterologous polypeptides to accomplish these other objectives are known in the art.

In some embodiments, the polypeptides for use in the methods of the invention are a component of a fusion protein that further comprises a heterologous polypeptide. In some embodiments, the heterologous polypeptide is an immunoglobulin constant domain. In some embodiments, the immunoglobulin constant domain is a heavy chain constant domain. In some embodiments, the heterologous polypeptide is an Fc fragment. In some embodiments the Fc is joined to the C-terminal end of the polypeptides for use in the methods of the invention. In some embodiments the fusion protein is a dimer. The invention further encompasses variants, analogs, or derivatives of polypeptide fragments as described above.

In one embodiment, the invention provides an isolated polypeptide fragment fused to a heterologous polypeptide to form a fusion protein, wherein said polypeptide fragment comprises an amino acid sequence identical to a reference amino acid sequence, except for up to one, two, three, four, five, ten, fifteen, or twenty individual amino acid substitutions. In another embodiment, at least one additional amino acid is added to the C-terminus of the polypeptide fragment. Exemplary amino acids that can be added to the polypeptide include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

As an alternative to expression of a fusion protein, a chosen heterologous polypeptide can be preformed and chemically conjugated to the polypeptides for use in the methods of the invention. In most cases, a chosen heterologous polypeptide will function similarly, whether fused or conjugated to the polypeptides for use in the methods of the invention. Therefore, in the following discussion of heterologous amino acid sequences, unless otherwise noted, it is to be understood that the heterologous sequence can be joined to the polypeptides for use in the methods of the invention in the form of a fusion protein or as a chemical conjugate.

Aptamers and antibodies and fragments thereof for use in the methods disclosed herein may also be recombinantly fused to a heterologous polypeptide at the N- or C-terminus or chemically conjugated (including covalent and non-covalent conjugations) to heterologous polypeptides. For example, sortilin aptamers and antibodies and fragments thereof may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

Polypeptides, aptamers, and antibodies and fragments thereof for use in the methods disclosed herein include derivatives that are modified, i.e., by the covalent attachment of any type of molecule such that covalent attachment does not prevent the polypeptide, aptamer, or antibody from inhibiting the biological function of sortilin or PGRN. For example, but not by way of limitation, the polypeptides, aptamers and antibodies and fragments thereof of the present invention may be modified, e.g., by glycosylation, acetylation, pegylation, phosphylation, phosphorylation, amidation, derivatization by known protecting/blocking groups, proteolytic cleavage, or linkage to a cellular ligand or other protein. Any of numerous chemical modifications may be carried out by known techniques, including, but not limited to specific chemical cleavage, acetylation, formylation, or metabolic synthesis of tunicamycin. Additionally, the derivative may contain one or more non-classical amino acids.

Polypeptides, aptamers and antibodies and fragments thereof for use in the methods disclosed herein can be composed of amino acids joined to each other by peptide bonds or modified peptide bonds, i.e., peptide isosteres, and may contain amino acids other than the 20 gene-encoded amino acids. Polypeptides, aptamers and antibodies and fragments thereof may be modified by natural processes, such as posttranslational processing, or by chemical modification techniques which are well known in the art. Such modifications are well described in basic texts and in more detailed monographs, as well as in a voluminous research literature. Modifications can occur anywhere in the polypeptide, aptamer or antibody or fragments thereof, including the peptide backbone, the amino acid side-chains and the amino or carboxyl termini, or on moieties such as carbohydrates. It will be appreciated that the same type of modification may be present in the same or varying degrees at several sites in a given polypeptide, aptamer or antibody or fragments thereof. Also, a given polypeptide, aptamer or antibody or fragments thereof may contain many types of modifications. Polypeptides, aptamers or antibodies or fragments thereof may be branched, for example, as a result of ubiquitination, and they may be cyclic, with or without branching. Cyclic, branched, and branched cyclic polypeptides, aptamers and antibodies or fragments thereof may result from posttranslational natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, Proteins—Structure And Molecular Properties, T. E. Creighton, W. H. Freeman and Company, New York 2nd Ed., (1993); Posttranslational Covalent Modification Of Proteins, B. C. Johnson, Ed., Academic Press, New York, pgs. 1-12 (1983); Seifter et al., Meth. Enzymol. 182:626-646 (1990); Rattan et al., Ann. NY Acad. Sci. 663:48-62 (1992)).

Pharmacologically active polypeptides such as the polypeptides, aptamers or antibodies or fragments thereof for use in the methods of the invention may exhibit rapid in vivo clearance, necessitating large doses to achieve therapeutically effective concentrations in the body. In addition, polypeptides smaller than about 60 kDa potentially undergo glomerular filtration, which sometimes leads to nephrotoxicity. Fusion or conjugation of relatively small polypeptides can be employed to reduce or avoid the risk of such nephrotoxicity. Various heterologous amino acid sequences, i.e., “carriers,” for increasing the in vivo stability, i.e., serum half-life, of therapeutic polypeptides are known. Examples include serum albumins such as, e.g., bovine serum albumin (BSA) or human serum albumin (HSA).

Due to its long half-life, wide in vivo distribution, and lack of enzymatic or immunological function, essentially full-length human serum albumin (HSA), or an HSA fragment, is commonly used as a heterologous polypeptide. Through application of methods and materials such as those taught in Yeh et al., Proc. Natl. Acad. Sci. USA, 89:1904-08 (1992) and Syed et al., Blood 89:3243-52 (1997), HSA can be used to form a fusion protein or polypeptide conjugate that displays pharmacological activity by virtue of the polypeptides for use in the methods of the invention while displaying significantly increased in vivo stability, e.g., 10-fold to 100-fold higher. The C-terminus of the HSA can be fused to the N-terminus of the polypeptides for use in the methods of the invention. Since HSA is a naturally secreted protein, the HSA signal sequence can be exploited to obtain secretion of the fusion protein into the cell culture medium when the fusion protein is produced in a eukaryotic, e.g., mammalian, expression system.

In certain embodiments, the polypeptides, aptamers, antibodies and antibody fragments thereof for use in the methods of the present invention further comprise a targeting moiety. Targeting moieties include a protein or a peptide which directs localization to a certain part of the body, for example, to the brain or compartments therein. In certain embodiments, the polypeptides, aptamers, antibodies or antibody fragments thereof for use in the methods of the present invention are attached or fused to a brain targeting moiety. The brain targeting moieties are attached covalently (e.g., direct, translational fusion, or by chemical linkage either directly or through a spacer molecule, which can be optionally cleavable) or non-covalently attached (e.g., through reversible interactions such as avidin:biotin, protein A:IgG, etc.). In other embodiments, the polypeptides, aptamers, antibodies or antibody fragments thereof for use in the methods of the present invention thereof are attached to one more brain targeting moieties. In additional embodiments, the brain targeting moiety is attached to a plurality of polypeptides, aptamers, antibodies or antibody fragments thereof for use in the methods of the present invention.

A brain targeting moiety associated with a polypeptide, aptamer, antibody or antibody fragment thereof for use in the methods of the present invention enhances brain delivery of such a polypeptide, antibody or antibody fragment thereof. A number of polypeptides have been described which, when fused to a protein or therapeutic agent, delivers the protein or therapeutic agent through the blood brain barrier (BBB). Non-limiting examples include the single domain antibody FC5 (Abulrob et al., J. Neurochem. 95:1201-1214 (2005)); mAB 83-14, a monoclonal antibody to the human insulin receptor (Pardridge et al., Pharmacol. Res. 12:807-816 (1995)); the B2, B6 and B8 peptides binding to the human transferrin receptor (hTfR) (Xia et al., J. Virol. 74:11359-11366 (2000)); the OX26 monoclonal antibody to the transferrin receptor (Pardridge et al., J. Pharmacol. Exp. Ther. 259:66-70 (1991)); diptheria toxin conjugates (see, e.g., Gaillard et al., International Congress Series 1277:185-198 (2005); and SEQ ID NOs: 1-18 of U.S. Pat. No. 6,306,365. The contents of the above references are incorporated herein by reference in their entirety.

Enhanced brain delivery of a polypeptide, aptamer, antibody or antibody fragment thereof for use in the methods of the present invention is determined by a number of means well established in the art. For example, administering to an animal a radioactively labeled polypeptide, aptamer, antibody or antibody fragment thereof linked to a brain targeting moiety; determining brain localization; and comparing localization with an equivalent radioactively labelled polypeptide, aptamer, antibody or antibody fragment thereof that is not associated with a brain targeting moiety. Other means of determining enhanced targeting are described in the above references.

Some embodiments of the invention employ a polypeptide fused to a hinge and Fc region, i.e., the C-terminal portion of an Ig heavy chain constant region, for use in the methods of the invention. In some embodiments, amino acids in the hinge region may be substituted with different amino acids. Exemplary amino acid substitutions for the hinge region according to these embodiments include substitutions of individual cysteine residues in the hinge region with different amino acids. Any different amino acid may be substituted for a cysteine in the hinge region. Amino acid substitutions for the amino acids of the polypeptides of the invention and the reference amino acid sequence can include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Typical amino acids to substitute for cysteines in the reference amino acid include alanine, serine, threonine, in particular, serine and alanine. Making such substitutions through engineering of a polynucleotide encoding the polypeptide fragment is well within the routine expertise of one of ordinary skill in the art.

Potential advantages of a PGRN polypeptide-Fc fusion or a sortilin polypeptide-Fc fusion include solubility, in vivo stability, and multivalency, e.g., dimerization. The Fc region used can be an IgA, IgD, or IgG Fc region (hinge-CH2-CH3). Alternatively, it can be an IgE or IgM Fc region (hinge-CH2-CH3-CH4). An IgG Fc region is generally used, e.g., an IgG1 Fc region or IgG4 Fc region. Materials and methods for constructing and expressing DNA encoding Fc fusions are known in the art and can be applied to obtain fusions without undue experimentation. Some embodiments of the invention employ a fusion protein such as those described in Capon et al., U.S. Pat. Nos. 5,428,130 and 5,565,335.

A signal sequence is a polynucleotide that encodes an amino acid sequence that can, in one example, initiate transport of a protein across the membrane of the endoplasmic reticulum. Signal sequences useful for constructing an immunofusin include antibody light chain signal sequences, e.g., antibody 14.18 (Gillies et al., J. Immunol. Meth. 125:191-202 (1989)), antibody heavy chain signal sequences, e.g., the MOPC141 antibody heavy chain signal sequence (Sakano et al., Nature 286:676-683 (1980)). Alternatively, other signal sequences can be used. See, e.g., Watson, Nucl. Acids Res. 12:5145-5164 (1984). A signal peptide can be cleaved in the lumen of the endoplasmic reticulum by signal peptidases. This results in the secretion of an immunofusin protein containing the Fc region and the polypeptide for use in the methods of the invention.

In some embodiments, the DNA sequence may encode a proteolytic cleavage site between the secretion cassette and the polypeptides for use in the methods of the invention. Such a cleavage site may provide, e.g., for the proteolytic cleavage of the encoded fusion protein, thus separating the Fc domain from the target protein. Useful proteolytic cleavage sites include amino acid sequences recognized by proteolytic enzymes such as trypsin, plasmin, thrombin, factor Xa, or enterokinase K.

The secretion cassette can be incorporated into a replicable expression vector. Useful vectors include linear nucleic acids, plasmids, phagemids, cosmids and the like. An exemplary expression vector is pdC, in which the transcription of the immunofusin DNA is placed under the control of the enhancer and promoter of the human cytomegalovirus. See, e.g., Lo et al., Biochim. Biophys. Acta 1088:217-224 (1991); and Lo et al., Protein Engineering 11:495-500 (1998). An appropriate host cell can be transformed or transfected with a DNA that encodes a polypeptide or polypeptide fragment for use in the methods of the invention and used for the expression and secretion of the polypeptide. Host cells that are typically used include immortal hybridoma cells, myeloma cells, 293 cells, Chinese hamster ovary (CHO) cells, Hela cells, and COS cells.

Fully intact, wild-type Fc regions display effector functions that normally are unnecessary and undesired in an Fc fusion protein used in the methods of the present invention. Therefore, certain binding sites typically are deleted from the Fc region during the construction of the secretion cassette. For example, since coexpression with the light chain is unnecessary, the binding site for the heavy chain binding protein, Bip (Hendershot et al., Immunol. Today 8:111-14 (1987)), is deleted from the CH2 domain of the Fc region of IgE, such that this site does not interfere with the efficient secretion of the immunofusin. Transmembrane domain sequences, such as those present in IgM, also are generally deleted.

The IgG1 Fc region is most often used. Alternatively, the Fc region of the other subclasses of immunoglobulin gamma (gamma-2, gamma-3 and gamma-4) can be used in the secretion cassette. The IgG1 Fc region of immunoglobulin gamma-1 is generally used in the secretion cassette and includes at least part of the hinge region, the CH2 region, and the CH3 region. In some embodiments, the Fc region of immunoglobulin gamma-1 is a CH2-deleted-Fc, which includes part of the hinge region and the CH3 region, but not the CH2 region. A CH2-deleted-Fc has been described by Gillies et al., Hum. Antibod. Hybridomas 1:47-54 (1990). In some embodiments, the Fc region of one of IgA, IgD, IgE, or IgM, is used.

PGRN polypeptide-Fc or sortilin polypeptide-Fc fusion proteins can be constructed in several different configurations. In one configuration the C-terminus of the PGRN or sortilin polypeptide is fused directly to the N-terminus of the Fc hinge moiety. In a slightly different configuration, a short polypeptide, e.g., 2-10 amino acids, is incorporated into the fusion between the N-terminus of the PGRN or sortilin polypeptide and the C-terminus of the Fc moiety. In the alternative configuration, the short polypeptide is incorporated into the fusion between the C-terminus of the PGRN or sortilin polypeptide and the N-terminus of the Fc moiety. If a sufficient portion of the hinge region is retained in the Fc moiety, the PGRN polypeptide-Fc or sortilin polypeptide-Fc fusion will dimerize, thus forming a divalent molecule. A homogeneous population of monomeric Fc fusions will yield monospecific, bivalent dimers. A mixture of two monomeric Fc fusions each having a different specificity will yield bispecific, bivalent dimers.

Any of a number of cross-linkers that contain a corresponding amino-reactive group and thiol-reactive group can be used to link a polypeptide or polypeptide fragment for use in the methods of the invention to serum albumin. Examples of suitable linkers include amine reactive cross-linkers that insert a thiol-reactive maleimide, e.g., SMCC, AMAS, BMPS, MBS, EMCS, SMPB, SMPH, KMUS, and GMBS. Other suitable linkers insert a thiol-reactive haloacetate group, e.g., SBAP, SIA, SIAB. Linkers that provide a protected or non-protected thiol for reaction with sulfhydryl groups to produce a reducible linkage include SPDP, SMPT, SATA, and SATP. Such reagents are commercially available (e.g., Pierce Chemical Company, Rockford, Ill.).

Polypeptides for use in the methods of the invention can be fused to a polypeptide tag. The term “polypeptide tag,” as used herein, is intended to mean any sequence of amino acids that can be attached to, connected to, or linked to a polypeptide for use in the methods of the invention and that can be used to identify, purify, concentrate or isolate the PGRN or sortilin polypeptide. The attachment of the polypeptide tag to the PGRN or sortilin polypeptide may occur, e.g., by constructing a nucleic acid molecule that comprises: (a) a nucleic acid sequence that encodes the polypeptide tag, and (b) a nucleic acid sequence that encodes a PGRN or a sortilin polypeptide. Exemplary polypeptide tags include, e.g., amino acid sequences that are capable of being post-translationally modified, e.g., amino acid sequences that are biotinylated. Other exemplary polypeptide tags include, e.g., amino acid sequences that are capable of being recognized and/or bound by an antibody (or fragment thereof) or other specific binding reagent. Polypeptide tags that are capable of being recognized by an antibody (or fragment thereof) or other specific binding reagent include, e.g., those that are known in the art as “epitope tags.” An epitope tag may be a natural or an artificial epitope tag. Natural and artificial epitope tags are known in the art, including, e.g., artificial epitopes such as FLAG, Strep, or poly-histidine peptides. FLAG peptides include the sequence Asp-Tyr-Lys-Asp-Asp-Asp-Asp-Lys (SEQ ID NO:22) or Asp-Tyr-Lys-Asp-Glu-Asp-Asp-Lys (SEQ ID NO:23) (Einhauer, A. and Jungbauer, A., J. Biochem. Biophys. Methods 49:1-3:455-465 (2001)). The Strep epitope has the sequence Ala-Trp-Arg-His-Pro-Gln-Phe-Gly-Gly (SEQ ID NO:24). The VSV-G epitope can also be used and has the sequence Tyr-Thr-Asp-Ile-Glu-Met-Asn-Arg-Leu-Gly-Lys (SEQ ID NO:25). Another artificial epitope is a poly-His sequence having six histidine residues (His-His-His-His-His-His (SEQ ID NO:26). Naturally-occurring epitopes include the influenza virus hemagglutinin (HA) sequence Tyr-Pro-Tyr-Asp-Val-Pro-Asp-Tyr-Ala-Ile-Glu-Gly-Arg (SEQ ID NO:27) recognized by the monoclonal antibody 12CA5 (Murray et al., Anal. Biochem. 229:170-179 (1995)) and the eleven amino acid sequence from human c-myc (Myc) recognized by the monoclonal antibody 9E10 (Glu-Gln-Lys-Leu-Leu-Ser-Glu-Glu-Asp-Leu-Asn (SEQ ID NO:28) (Manstein et al., Gene 162:129-134 (1995)). Another useful epitope is the tripeptide Glu-Glu-Phe which is recognized by the monoclonal antibody YL 1/2 (Stammers et al., FEBS Lett. 283:298-302(1991)).

In certain embodiments, the PGRN or sortilin polypeptide and the polypeptide tag may be connected via a linking amino acid sequence. As used herein, a “linking amino acid sequence” may be an amino acid sequence that is capable of being recognized and/or cleaved by one or more proteases. Amino acid sequences that can be recognized and/or cleaved by one or more proteases are known in the art. Exemplary amino acid sequences are those that are recognized by the following proteases: factor VIIa, factor IXa, factor Xa, APC, t-PA, u-PA, trypsin, chymotrypsin, enterokinase, pepsin, cathepsin B,H,L,S,D, cathepsin G, renin, angiotensin converting enzyme, matrix metalloproteases (collagenases, stromelysins, gelatinases), macrophage elastase, Cir, and Cis. The amino acid sequences that are recognized by the aforementioned proteases are known in the art. Exemplary sequences recognized by certain proteases can be found, e.g., in U.S. Pat. No. 5,811,252.

Polypeptide tags can facilitate purification using commercially available chromatography media.

In some embodiments of the invention, a polypeptide fusion construct is used to enhance the production of a PGRN or sortilin polypeptide in bacteria. In such constructs a bacterial protein normally expressed and/or secreted at a high level is employed as the N-terminal fusion partner of a polypeptide or polypeptide fragment for use in the methods of the invention. See, e.g., Smith et al., Gene 67:31-40 (1988); Hopp et al., Biotechnology 6:1204-1210 (1988); La Vallie et al., Biotechnology 11:187-193 (1993).

By fusing a polypeptide at the amino and carboxy termini of a suitable fusion partner, bivalent or tetravalent forms of a polypeptide or polypeptide fragment for use in the methods of the invention can be obtained. For example, a PGRN or a sortilin polypeptide can be fused to the amino and carboxy termini of an Ig moiety to produce a bivalent monomeric polypeptide containing two PGRN or two sortilin polypeptides. Upon dimerization of two of these monomers, by virtue of the Ig moiety, a tetravalent form of a PGRN or a sortilin polypeptide is obtained. Such multivalent forms can be used to achieve increased binding affinity for the target. Multivalent forms of a PGRN or a sortilin polypeptide or polypeptide fragment of the invention also can be obtained by placing PGRN or sortilin polypeptides in tandem to form concatamers, which can be employed alone or fused to a fusion partner such as Ig or HSA.

Conjugated Polymers (Other than Polypeptides)

Some embodiments of the invention involve a polypeptide or polypeptide fragment for use in the methods of the invention wherein one or more polymers are conjugated (covalently linked) to the polypeptide. Examples of polymers suitable for such conjugation include polypeptides (discussed above), sugar polymers, and polyalkylene glycol chains. Typically, but not necessarily, a polymer is conjugated to the polypeptide or polypeptide fragment for use in the methods of the invention for the purpose of improving one or more of the following: solubility, stability, or bioavailability.

The class of polymer generally used for conjugation to a polypeptide or polypeptide fragment for use in the methods of the invention is a polyalkylene glycol. Polyethylene glycol (PEG) is most frequently used. PEG moieties, e.g., 1, 2, 3, 4 or 5 PEG polymers, can be conjugated to each PGRN or sortilin polypeptide to increase serum half life, as compared to the PGRN or sortilin polypeptide alone. PEG moieties are non-antigenic and essentially biologically inert. PEG moieties used in the practice of the invention may be branched or unbranched.

The number of PEG moieties attached to the PGRN or sortilin polypeptide and the molecular weight of the individual PEG chains can vary. In general, the higher the molecular weight of the polymer, the fewer polymer chains attached to the polypeptide. Usually, the total polymer mass attached to a PGRN or a sortilin polypeptide or polypeptide fragment is from 20 kDa to 40 kDa. Thus, if one polymer chain is attached, the molecular weight of the chain is generally 20-40 kDa. If two chains are attached, the molecular weight of each chain is generally 10-20 kDa. If three chains are attached, the molecular weight is generally 7-14 kDa.

The polymer, e.g., PEG, can be linked to a PGRN or sortilin polypeptide through any suitable, exposed reactive group on the polypeptide. The exposed reactive group(s) can be, e.g., an N-terminal amino group or the epsilon amino group of an internal lysine residue, or both. An activated polymer can react and covalently link at any free amino group on the PGRN or sortilin polypeptide. Free carboxylic groups, suitably activated carbonyl groups, hydroxyl, guanidyl, imidazole, oxidized carbohydrate moieties and mercapto groups of the PGRN or sortilin polypeptide (if available) also can be used as reactive groups for polymer attachment.

In a conjugation reaction, from about 1.0 to about 10 moles of activated polymer per mole of polypeptide, depending on polypeptide concentration, is typically employed. Usually, the ratio chosen represents a balance between maximizing the reaction while minimizing side reactions (often non-specific) that can impair the desired pharmacological activity of the PGRN or sortilin polypeptide moiety. Preferably, at least 50% of the biological activity (as demonstrated, e.g., in any of the assays described herein or known in the art) of the PGRN or sortilin polypeptide is retained, and most preferably nearly 100% is retained.

The polymer can be conjugated to the PGRN or sortilin polypeptide using conventional chemistry. For example, a polyalkylene glycol moiety can be coupled to a lysine epsilon amino group of the PGRN or sortilin polypeptide. Linkage to the lysine side chain can be performed with an N-hydroxylsuccinimide (NHS) active ester such as PEG succinimidyl succinate (SS-PEG) and succinimidyl propionate (SPA-PEG). Suitable polyalkylene glycol moieties include, e.g., carboxymethyl-NHS and norleucine-NHS, SC. These reagents are commercially available. Additional amine-reactive PEG linkers can be substituted for the succinimidyl moiety. These include, e.g., isothiocyanates, nitrophenylcarbonates (PNP), epoxides, benzotriazole carbonates, SC-PEG, tresylate, aldehyde, epoxide, carbonylimidazole and PNP carbonate. Conditions are usually optimized to maximize the selectivity and extent of reaction. Such optimization of reaction conditions is within ordinary skill in the art.

PEGylation can be carried out by any of the PEGylation reactions known in the art. See, e.g., Focus on Growth Factors 3: 4-10 (1992) and European patent applications EP 0 154 316 and EP 0 401 384. PEGylation may be carried out using an acylation reaction or an alkylation reaction with a reactive polyethylene glycol molecule (or an analogous reactive water-soluble polymer).

PEGylation by acylation generally involves reacting an active ester derivative of polyethylene glycol. Any reactive PEG molecule can be employed in the PEGylation. PEG esterified to N-hydroxysuccinimide (NHS) is a frequently used activated PEG ester. As used herein, “acylation” includes without limitation the following types of linkages between the therapeutic protein and a water-soluble polymer such as PEG: amide, carbamate, urethane, and the like. See, e.g., Bioconjugate Chem. 5:133-140 (1994). Reaction parameters are generally selected to avoid temperature, solvent, and pH conditions that would damage or inactivate the PORN or sortilin polypeptide.

Generally, the connecting linkage is an amide and typically at least 95% of the resulting product is mono-, di- or tri-PEGylated. However, some species with higher degrees of PEGylation may be formed in amounts depending on the specific reaction conditions used. Optionally, purified PEGylated species are separated from the mixture, particularly unreacted species, by conventional purification methods, including, e.g., dialysis, salting-out, ultrafiltration, ion-exchange chromatography, gel filtration chromatography, hydrophobic exchange chromatography, and electrophoresis.

PEGylation by alkylation generally involves reacting a terminal aldehyde derivative of PEG with a polypeptide or polypeptide fragment for use in the methods of the invention in the presence of a reducing agent. In addition, one can manipulate the reaction conditions to favor PEGylation substantially only at the N-terminal amino group of the PGRN or sortilin polypeptide, i.e., a mono-PEGylated protein. In either case of mono-PEGylation or poly-PEGylation, the PEG groups are typically attached to the protein via a —CH₂—NH— group. With particular reference to the —CH₂— group, this type of linkage is known as an “alkyl” linkage.

Derivatization via reductive alkylation to produce an N-terminally targeted mono-PEGylated product exploits differential reactivity of different types of primary amino groups (lysine versus the N-terminal) available for derivatization. The reaction is performed at a pH that allows one to take advantage of the pKa differences between the epsilon-amino groups of the lysine residues and that of the N-terminal amino group of the protein. By such selective derivatization, attachment of a water-soluble polymer that contains a reactive group, such as an aldehyde, to a protein is controlled: the conjugation with the polymer takes place predominantly at the N-terminus of the protein and no significant modification of other reactive groups, such as the lysine side chain amino groups, occurs.

The polymer molecules used in both the acylation and alkylation approaches are selected from among water-soluble polymers. The polymer selected is typically modified to have a single reactive group, such as an active ester for acylation or an aldehyde for alkylation, so that the degree of polymerization may be controlled as provided for in the present methods. An exemplary reactive PEG aldehyde is polyethylene glycol propionaldehyde, which is water stable, or mono C₁-C₁₀ alkoxy or aryloxy derivatives thereof (see, e.g., U.S. Pat. No. 5,252,714). The polymer may be branched or unbranched. For the acylation reactions, the polymer(s) selected typically have a single reactive ester group. For reductive alkylation, the polymer(s) selected typically have a single reactive aldehyde group. Generally, the water-soluble polymer will not be selected from naturally occurring glycosyl residues, because these are usually made more conveniently by mammalian recombinant expression systems.

Methods for preparing a PEGylated polypeptide for use in the methods of the invention generally includes the steps of (a) reacting a polypeptide or polypeptide fragment for use in the methods of the invention with polyethylene glycol (such as a reactive ester or aldehyde derivative of PEG) under conditions whereby the molecule becomes attached to one or more PEG groups, and (b) obtaining the reaction product(s). In general, the optimal reaction conditions for the acylation reactions will be determined case-by-case based on known parameters and the desired result. For example, a larger the ratio of PEG to protein, generally leads to a greater the percentage of poly-PEGylated product.

Reductive alkylation to produce a substantially homogeneous population of mono-polymer/PGRN polypeptide or sortilin polypeptide generally includes the steps of: (a) reacting a PGRN or a sortilin polypeptide or polypeptide fragment for use in the methods of the invention with a reactive PEG molecule under reductive alkylation conditions, at a pH suitable to permit selective modification of the N-terminal amino group of PGRN or sortilin; and (b) obtaining the reaction product(s).

For a substantially homogeneous population of mono-polymer/PGRN polypeptide or sortilin polypeptide, the reductive alkylation reaction conditions are those that permit the selective attachment of the water-soluble polymer moiety to the N-terminus of a polypeptide or polypeptide fragment for use in the methods of the invention. Such reaction conditions generally provide for pKa differences between the lysine side chain amino groups and the N-terminal amino group. For purposes of the present invention, the pH is generally in the range of 3-9, typically 3-6.

Polypeptides for use in the methods of the invention can include a tag, e.g., a moiety that can be subsequently released by proteolysis. Thus, the lysine moiety can be selectively modified by first reacting a His-tag modified with a low-molecular-weight linker such as Traut's reagent (Pierce Chemical Company, Rockford, Ill.) which will react with both the lysine and N-terminus, and then releasing the His tag. The polypeptide will then contain a free SH group that can be selectively modified with a PEG containing a thiol-reactive head group such as a maleimide group, a vinylsulfone group, a haloacetate group, or a free or protected SH.

Traut's reagent can be replaced with any linker that will set up a specific site for PEG attachment. For example, Traut's reagent can be replaced with SPDP, SMPT, SATA, or SATP (Pierce Chemical Company, Rockford, Ill.). Similarly one could react the protein with an amine-reactive linker that inserts a maleimide (for example SMCC, AMAS, BMPS, MBS, EMCS, SMPB, SMPH, KMUS, or GMBS), a haloacetate group (SBAP, SIA, SIAB), or a vinylsulfone group and react the resulting product with a PEG that contains a free SH.

In some embodiments, the polyalkylene glycol moiety is coupled to a cysteine group of a polypeptide for use in the methods of the invention. Coupling can be effected using, e.g., a maleimide group, a vinylsulfone group, a haloacetate group, or a thiol group.

Optionally, the polypeptides for use in the methods of the invention are conjugated to the polyethylene-glycol moiety through a labile bond. The labile bond can be cleaved in, e.g., biochemical hydrolysis, proteolysis, or sulfhydryl cleavage. For example, the bond can be cleaved under in vivo (physiological) conditions.

The reactions may take place by any suitable method used for reacting biologically active materials with inert polymers, generally at about pH 5-8, e.g., pH 5, 6, 7, or 8, if the reactive groups are on the alpha amino group at the N-terminus. Generally the process involves preparing an activated polymer and thereafter reacting the protein with the activated polymer to produce the soluble protein suitable for formulation.

### Nucleic Acid Molecules

The methods of the present invention provide a nucleic acid that encodes a polypeptide for use in the methods of the invention, including the polypeptides of any one of SEQ ID NOs:21, 30, 32, 34, or 36. As used herein, “nucleic acid” means genomic DNA, cDNA, mRNA and antisense molecules, as well as nucleic acids based on alternative backbones or including alternative bases whether derived from natural sources or synthesized. In some embodiments, the nucleic acid further comprises a transcriptional promoter and optionally a signal sequence each of which is operably linked to the nucleotide sequence encoding the polypeptides for use in the methods of the invention.

In some embodiments, the invention provides a nucleic acid encoding a fusion protein for use in the methods of the invention. In some embodiments, the nucleic acid encoding a fusion protein further comprises a transcriptional promoter and optionally a signal sequence. In some embodiments, the nucleotide sequence further encodes an immunoglobulin constant region. In some embodiments, the immunoglobulin constant region is a heavy chain constant region. In some embodiments, the nucleotide sequence further encodes an immunoglobulin heavy chain constant region joined to a hinge region. In some embodiments the nucleic acid further encodes Fc. In some embodiments the fusion proteins comprise an Fc fragment.

The nucleic acids that encode proteins for use in the methods of the present invention may further be modified so as to contain a detectable label for diagnostic and probe purposes. A variety of such labels are known in the art and can readily be employed with the encoding molecules herein described. Suitable labels include, but are not limited to, biotin, radiolabeled nucleotides and the like. A skilled artisan can employ any of the art known labels to obtain a labeled encoding nucleic acid molecule.

In some embodiments, polynucleotides are included that hybridize under moderately stringent or high stringency conditions to the noncoding strand, or complement, of a polynucleotide that encodes any one of the polypeptides for use in the methods of the invention. Stringent conditions are known to those skilled in the art and can be found in CURRENT PROTOCOLS IN MOLECULAR BIOLOGY, John Wiley & Sons, N.Y. (1989), 6.3.1-6.3.6.

Polynucleotide Antagonists

Specific embodiments comprise polynucleotide antagonists which modulate the activity of PGRN, the activity of sortilin, or the PGRN-sortilin interaction. Polynucleotide antagonists include, but are not limited to antisense molecules, ribozymes, siRNA, shRNA and RNAi. Typically, such binding molecules are separately administered to the animal (see, e.g., O'Connor, J. Neurochem. 56:560 (1991)), but such binding molecules may also be expressed in vivo from polynucleotides taken up by a host cell and expressed in vivo. See also, Oligodeoxynucleotides as Antisense Inhibitors of Gene Expression, CRC Press, Boca Raton, Fla. (1988).

Expression of the PGRN or sortilin gene can, in some embodiments, be inhibited using RNA interference (“RNAi”). RNAi refers to the expression of an RNA which interferes with the expression of the targeted mRNA. RNAi is a phenomenon in which the introduction of double-stranded RNA (dsRNA) into a cell causes degradation of the homologous mRNA. An “RNAi nucleic acid” as used herein is a nucleic acid sequence generally shorter than 50 nucleotides in length, that causes gene silencing at the mRNA level.

For example, in mammalian cells, introduction of long dsRNA (>30 nucleotides) can initiate a potent antiviral response, exemplified by nonspecific inhibition of protein synthesis and RNA degradation. RNA interference provides a mechanism of gene silencing at the mRNA level. In recent years, RNAi has become an endogenous and potent gene-specific silencing technique that uses double-stranded RNAs (dsRNA) to mark a particular transcript for degradation in vivo. It also offers an efficient and broadly applicable approach for gene knock-out. In addition, RNAi technology can be used for therapeutic purposes. For example, RNAi targeting Fas-mediated apoptosis has been shown to protect mice from fulminant hepatitis. RNAi technology has been disclosed in numerous publications, such as U.S. Pat. Nos. 5,919,619, 6,506,559 and PCT Publication Nos. WO99/14346, WO01/70949, WO01/36646, WO00/63364, WO00/44895, WO01/75164, WO01/92513, WO01/68836 and WO01/29058.

Specifically, the RNAi silences a targeted gene via interacting with the specific mRNA (e.g., PGRN or sortilin) through a siRNA (short interfering RNA). The ds RNA complex is then targeted for degradation by the cell. Additional RNAi molecules include Short hairpin RNA (shRNA); also short interfering hairpin. The shRNA molecule contains sense and antisense sequences from a target gene connected by a loop. The shRNA is transported from the nucleus into the cytoplasm, it is degraded along with the mRNA. Pol III or U6 promoters can be used to express RNAs for RNAi. A sequence capable of inhibiting gene expression by RNA interference can have any length. For instance, the sequence can have at least 10, 15, 20, 25, 30, 35, 40, 45, 50, 100 or more consecutive nucleotides. The sequence can be dsRNA or any other type of polynucleotide, provided that the sequence can form a functional silencing complex to degrade the target mRNA transcript.

RNAi is mediated by double stranded RNA (dsRNA) molecules that have sequence-specific homology to their “target” mRNAs (Caplen et al., Proc Natl Acad Sci USA 98:9742-9747, 2001). Biochemical studies in Drosophila cell-free lysates indicates that the mediators of RNA-dependent gene silencing are 18-25 nucleotide “small interfering” RNA duplexes (siRNAs). Accordingly, siRNA molecules are advantageously used in the methods of the present invention. siRNAs can be produced endogenously by degradation of longer dsRNA molecules by an RNase III-related nuclease called Dicer. (Bernstein et al., Nature 409:363-366, 2001). siRNAs can also be introduced into a cell exogenously, or by transcription of an expression construct. Once formed, the siRNAs assemble with protein components into endoribonuclease-containing complexes known as RNA-induced silencing complexes (RISCs). An ATP-generated unwinding of the siRNA activates the RISCs, which in turn target the complementary mRNA transcript by Watson-Crick base-pairing. Without wishing to be bound by any particular theory, it is believed that a RISC is guided to a target mRNA, where the siRNA duplex interacts sequence-specifically to mediate cleavage in a catalytic fashion (Bernstein et al., Nature 409:363-366, 2001; Boutla et al., Curr Biol 11:1776-1780, 2001). Cleavage of the mRNA takes place near the middle of the region bound by the siRNA strand. This sequence specific mRNA degradation results in gene silencing.

RNAi has been used to analyze gene function and to identify essential genes in mammalian cells (Elbashir et al., Methods 26:199-213, 2002; Harborth et al., J Cell Sci 114:4557-4565, 2001), including by way of non-limiting example neurons (Krichevsky et al., Proc Natl Acad Sci USA 99:11926-11929, 2002). RNAi is also being evaluated for therapeutic modalities, such as inhibiting or blocking the infection, replication and/or growth of viruses, including without limitation poliovirus (Gitlin et al., Nature 418:379-380, 2002) and HIV (Capodici et al., J Immunol 169:5196-5201, 2002), and reducing expression of oncogenes (e.g., the bcr-abl gene; Scherr et al., Blood 101:1566-1569 (2003)). RNAi has been used to modulate gene expression in mammalian (mouse) and amphibian (Xenopus) embryos (respectively, Calegari et al., Proc Natl Acad Sci USA 99:14236-14240, 2002; and Zhou, et al., Nucleic Acids Res 30:1664-1669, 2002), and in postnatal mice (Lewis et al., Nat Genet 32:107-108, 2002), and to reduce transgene expression in adult transgenic mice (McCaffrey et al., Nature 418:38-39, 2002). Methods have been described for determining the efficacy and specificity of siRNAs in cell culture and in vivo (see, e.g., Bertrand et al., Biochem Biophys Res Commun 296:1000-1004, 2002; Lassus et al., Sci STKE 2002(147):PL13, 2002; and Leirdal et al., Biochem Biophys Res Commun 295:744-748, 2002).

Molecules that mediate RNAi, including without limitation siRNA, can be produced in vitro by chemical synthesis (Hohjoh, FEBS Lett 521:195-199 (2002)), hydrolysis of dsRNA (Yang et al., Proc Natl Acad Sci USA 99:9942-9947 (2002)), by in vitro transcription with T7 RNA polymerase (Donzeet et al., Nucleic Acids Res 30:e46, 2002; Yu et al., Proc Natl Acad Sci USA 99:6047-6052, 2002), and by hydrolysis of double-stranded RNA using a nuclease such as E. coli RNase III (Yang et al., Proc Natl Acad Sci USA 99:9942-9947 (2002)).

siRNA molecules may also be formed by annealing two oligonucleotides to each other, typically have the following general structure, which includes both double-stranded and single-stranded portions:

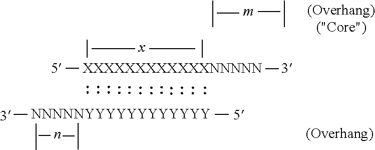

Wherein N, X and Y are nucleotides; X hydrogen bonds to Y; “:” signifies a hydrogen bond between two bases; x is a natural integer having a value between 1 and about 100; and m and n are whole integers having, independently, values between 0 and about 100. In some embodiments, N, X and Y are independently A, G, C and T or U. Non-naturally occurring bases and nucleotides can be present, particularly in the case of synthetic siRNA (i.e., the product of annealing two oligonucleotides). The double-stranded central section is called the “core” and has base pairs (bp) as units of measurement; the single-stranded portions are overhangs, having nucleotides (nt) as units of measurement. The overhangs shown are 3′ overhangs, but molecules with 5′ overhangs are also within the scope of the invention. Also within the scope of the invention are siRNA molecules with no overhangs (i.e., m=0 and n=0), and those having an overhang on one side of the core but not the other (e.g., m=0 and n>1, or vice-versa).

Paddison et al. (Genes & Dev. 16:948-958 (2002)) have used small RNA molecules folded into hairpins as a means to effect RNAi. Accordingly, such short hairpin RNA (shRNA) molecules are also advantageously used in the methods of the invention. The length of the stem and loop of functional shRNAs varies; stem lengths can range anywhere from about 25 to about 30 nt, and loop size can range between 4 to about 25 nt without affecting silencing activity. While not wishing to be bound by any particular theory, it is believed that these shRNAs resemble the dsRNA products of the DICER RNase and, in any event, have the same capacity for inhibiting expression of a specific gene.

Chemically synthesizing nucleic acid molecules with modifications (base, sugar and/or phosphate) can prevent their degradation by serum ribonucleases, which can increase their potency (see, e.g., International Publication No. WO 92/07065; Perrault, et al., Nature 344:565 (1990); Pieken et al., Science 253:314 (1991); Usman and Cedergren, TIBS 17:334-339 (1992); International Publication No. WO 93/15187; and International Publication No. WO 91/03162; U.S. Pat. No. 5,334,711; U.S. Pat. No. 6,300,074; all of which are incorporated by reference herein). All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-O-allyl, 2′-H, nucleotide base modifications (see generally, Usman and Cedergren, TIBS 17:334-339 (1992); Usman et al., Nucleic Acids Symp. Ser. 31:163 (1994); Burgin et al., Biochemistry 35:14090 (1996)). Sugar modification of nucleic acid molecules have been extensively described in the art (see International Publication PCT No. WO 92/07065; Perrault et al., Nature 344:565-568 (1990); Pieken et al., Science 253:314-317 (1991); Usman and Cedergren, supra; International Publication PCT No. WO 93/15187; U.S. Pat. No. 5,334,711 and Beigelman et al., J. Biol. Chem. 270:25702 (1995); International PCT publication No. WO 97/26270; U.S. Pat. No. 5,716,824; U.S. Pat. No. 5,627,053; International PCT Publication No. WO 98/13526; Karpeisky et al., Tetrahedron Lett. 39:1131 (1998); Earnshaw and Gait, Biopolymers (Nucleic Acid Sciences) 48:39-55 (1998); Verma and Eckstein, Annu. Rev. Biochem. 67:99-134 (1998); and Burlina et al., Bioorg. Med. Chem. 5:1999-2010 (1997); all of the references are hereby incorporated in their totality by reference herein). Such publications describe general methods and strategies to determine the location of incorporation of sugar, base and/or phosphate modifications and the like into nucleic acid molecules without modulating catalysis. In view of such teachings, similar modifications can be used as described herein to modify the siRNA nucleic acid molecules of the instant invention so long as the ability of siRNA to promote RNAi in cells is not significantly inhibited.

The invention features modified siRNA molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, Nucleic Acid Analogues: Synthesis and Properties, in Modern Synthetic Methods, VCH, 331-417 (1995), and Mesmaeker et al., Novel Backbone Replacements for Oligonucleotides, in Carbohydrate Modifications in Antisense Research, ACS, 24-39 (1994).

While chemical modification of oligonucleotide internucleotide linkages with phosphorothioate and/or 5′-methylphosphonate linkages improves stability, excessive modifications can cause some toxicity or decreased activity. Therefore, when designing nucleic acid molecules, the amount of these internucleotide linkages should be minimized. The reduction in the concentration of these linkages should lower toxicity, resulting in increased efficacy and higher specificity of these molecules.

siRNA molecules having chemical modifications that maintain or enhance activity are provided. Such a nucleic acid is also generally more resistant to nucleases than an unmodified nucleic acid. Accordingly, the in vitro and/or in vivo activity should not be significantly lowered. In cases in which modulation is the goal, therapeutic nucleic acid molecules delivered exogenously should optimally be stable within cells until translation of the target RNA has been modulated long enough to reduce the levels of the undesirable protein. This period of time varies between hours to days depending upon the disease state. Improvements in the chemical synthesis of RNA and DNA (Wincott et al., Nucleic Acids Res. 23:2677 (1995); Caruthers et al., Methods in Enzymology 211:3-19 (1992) (incorporated by reference herein)) have expanded the ability to modify nucleic acid molecules by introducing nucleotide modifications to enhance their nuclease stability, as described above.

Polynucleotides for use in the methods of the present invention can include one or more (e.g., about 1, 2, 3, 4, 5, 6, 7, 8, 9, 10, or more) G-clamp nucleotides. A G-clamp nucleotide is a modified cytosine analog wherein the modifications confer the ability to hydrogen bond both Watson-Crick and Hoogsteen faces of a complementary guanine within a duplex, see, e.g., Lin and Matteucci, J. Am. Chem. Soc. 120:8531-8532 (1998). A single G-clamp analog substitution within an oligonucleotide can result in substantially enhanced helical thermal stability and mismatch discrimination when hybridized to complementary oligonucleotides. The inclusion of such nucleotides in polynucleotides of the invention results in both enhanced affinity and specificity to nucleic acid targets, complementary sequences, or template strands. Polynucleotides for use in the methods of the present invention can also include one or more (e.g., about 1, 2, 3, 4, 5, 6, 7, 8, 9, 10, or more) LNA “locked nucleic acid” nucleotides such as a 2′,4′-C methylene bicyclo nucleotide (see, e.g., International PCT Publication Nos. WO 00/66604 and WO 99/14226).

The present invention also features conjugates and/or complexes of siRNA molecules for use in the methods of the invention. Such conjugates and/or complexes can be used to facilitate delivery of siRNA molecules into a biological system, such as a cell. The conjugates and complexes provided by the instant invention can impart therapeutic activity by transferring therapeutic compounds across cellular membranes, altering the pharmacokinetics, and/or modulating the localization of nucleic acid molecules of the invention. The methods of the present invention encompasses the design and synthesis of novel conjugates and complexes for the delivery of molecules, including, but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery and/or localization of nucleic acid molecules of the invention into a number of cell types originating from different tissues, in the presence or absence of serum (see U.S. Pat. No. 5,854,038). Conjugates of the molecules described herein can be attached to biologically active molecules via linkers that are biodegradable, such as biodegradable nucleic acid linker molecules.

Therapeutic polynucleotides (e.g., siRNA molecules) delivered exogenously optimally are stable within cells until reverse transcription of the RNA has been modulated long enough to reduce the levels of the RNA transcript. The nucleic acid molecules are resistant to nucleases in order to function as effective intracellular therapeutic agents. Improvements in the chemical synthesis of nucleic acid molecules described in the instant invention and in the art have expanded the ability to modify nucleic acid molecules by introducing nucleotide modifications to enhance their nuclease stability as described above.

Use of the polynucleotide-based molecules in the methods of the invention will lead to better treatment of the disease progression by affording the possibility of combination therapies (e.g., multiple siRNA molecules targeted to different genes; nucleic acid molecules coupled with known small molecule modulators; or intermittent treatment with combinations of molecules, including different motifs and/or other chemical or biological molecules). The treatment of subjects with siRNA molecules can also include combinations of different types of nucleic acid molecules, such as enzymatic nucleic acid molecules (ribozymes), allozymes, antisense, 2,5-A oligoadenylate, decoys, aptamers etc.

In another aspect, an siRNA molecule for use in the methods of the invention can comprise one or more 5′ and/or a 3′-cap structures, for example on only the sense siRNA strand, antisense siRNA strand, or both siRNA strands.

By “cap structure” is meant chemical modifications, which have been incorporated at either terminus of the oligonucleotide (see, e.g., U.S. Pat. No. 5,998,203, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples: the 5′-cap is selected from the group comprising inverted abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moeity.

The 3′-cap can be selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, Tetrahedron 49:1925 (1993); incorporated by reference herein).

Antisense technology can be used to control gene expression through antisense DNA or RNA, or through triple-helix formation. Antisense techniques are discussed for example, in Okano, J. Neurochem. 56:560 (1991); Oligodeoxynucleotides as Antisense Inhibitors of Gene Expression, CRC Press, Boca Raton, Fla. (1988). Triple helix formation is discussed in, for instance, Lee et al., Nucleic Acids Research 6:3073 (1979); Cooney et al., Science 241:456 (1988); and Dervan et al., Science 251:1300 (1991). The methods are based on binding of a polynucleotide to a complementary DNA or RNA.

For example, the 5′ coding portion of a polynucleotide that encodes PGRN or sortilin may be used to design an antisense RNA oligonucleotide of from about 10 to 40 base pairs in length. A DNA oligonucleotide is designed to be complementary to a region of the gene involved in transcription thereby preventing transcription and the production of the target protein. The antisense RNA oligonucleotide hybridizes to the mRNA in vivo and blocks translation of the mRNA molecule into the target polypeptide.

In one embodiment, antisense nucleic acids specific for the PGRN or sortilin gene are produced intracellularly by transcription from an exogenous sequence. For example, a vector or a portion thereof, is transcribed, producing an antisense nucleic acid (RNA). Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in vertebrate cells. Expression of the antisense molecule, can be by any promoter known in the art to act in vertebrate, preferably human cells, such as those described elsewhere herein.

Absolute complementarity of an antisense molecule, although preferred, is not required. A sequence complementary to at least a portion of an RNA encoding PGRN or sortilin, means a sequence having sufficient complementarity to be able to hybridize with the RNA, forming a stable duplex; or triplex formation may be assayed. The ability to hybridize will depend on both the degree of complementarity and the length of the antisense nucleic acid. Generally, the larger the hybridizing nucleic acid, the more base mismatches it may contain and still form a stable duplex (or triplex as the case may be). One skilled in the art can ascertain a tolerable degree of mismatch by use of standard procedures to determine the melting point of the hybridized complex.

Oligonucleotides that are complementary to the 5′ end of a messenger RNA, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., Nature 372:333-335 (1994). Thus, oligonucleotides complementary to either the 5′- or 3′-non-translated, non-coding regions could be used in an antisense approach to inhibit translation of PGRN or sortilin. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides, or at least 50 nucleotides.

Polynucleotides for use in the methods disclosed herein, including aptamers described below, can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., Proc. Natl. Acad. Sci. U.S.A. 86:6553-6556 (1989); Lemaitre et al., Proc. Natl. Acad. Sci. 84:648-652 (1987)); PCT Publication No. WO88/09810) or the blood-brain barrier (see, e.g., PCT Publication No. WO89/10134), hybridization-triggered cleavage agents (see, e.g., Krol et al., BioTechniques 6:958-976 (1988)) or intercalating agents (see, e.g., Zon, Pharm. Res. 5:539-549(1988)). To this end, the oligonucleotide may be conjugated to another molecule, e.g., a peptide, hybridization triggered cross-linking agent, transport agent, hybridization-triggered cleavage agent, etc.

An antisense oligonucleotide for use in the methods disclosed herein may comprise at least one modified base moiety which is selected from the group including, but not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-isopentenyladenine, 1-methylguanine, 1-methylinosine, 2,2-dimethylguanine, 2-methyladenine, 2-methylguanine, 3-methylcytosine, 5-methylcytosine, N-6-adenine, 7-methylguanine, 5-methylaminomethyluracil, 5-methoxyaminomethyl-2-thiouracil, beta-D-mannosylqueosine, 5′methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3(3-amino-3-N2-carboxypropyl) uracil, (acp3)w, and 2,6-diaminopurine.

An antisense oligonucleotide for use in the methods disclosed herein may also comprise at least one modified sugar moiety selected from the group including, but not limited to, arabinose, 2-fluoroarabinose, xylulose, and hexose. In yet another embodiment, an antisense oligonucleotide for use in the methods disclosed herein comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, an antisense oligonucleotide for use in the methods disclosed herein is an α-anomeric oligonucleotide. An α-anomeric oligonucleotide forms specific double-stranded hybrids with complementary RNA in which, contrary to the usual situation, the strands run parallel to each other (Gautier et al., Nucl. Acids Res. 15:6625-6641(1987)). The oligonucleotide is a 2′-0-methylribonucleotide (Inoue et al., Nucl. Acids Res. 15:6131-6148(1987)), or a chimeric RNA-DNA analogue (Inoue et al., FEBS Lett. 215:327-330(1987)).

Polynucleotides for use in the methods of the invention, including aptamers, may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al., Nucl. Acids Res. 16:3209 (1988), and methylphosphonate oligonucleotides can be prepared by use of controlled pore glass polymer supports (Sarin et al., Proc. Natl. Acad. Sci. U.S.A. 85:7448-7451(1988)).

Polynucleotide compositions for use in the methods disclosed herein further include catalytic RNA, or a ribozyme (see, e.g., PCT International Publication WO 90/11364; Sarver et al., Science 247:1222-1225 (1990)). The use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). Preferably, the ribozyme is engineered so that the cleavage recognition site is located near the 5′ end of the target mRNA, i.e., to increase efficiency and minimize the intracellular accumulation of non-functional mRNA transcripts.

As in the antisense approach, ribozymes for use in the diagnostic and therapeutic methods disclosed herein can be composed of modified oligonucleotides (e.g., for improved stability, targeting, etc.) and may be delivered to cells which express PGRN or sortilin in vivo. DNA constructs encoding the ribozyme may be introduced into the cell in the same manner as described above for the introduction of antisense encoding DNA. A preferred method of delivery involves using a DNA construct “encoding” the ribozyme under the control of a strong constitutive promoter, such as, for example, pol III or pol II promoter, so that transfected cells will produce sufficient quantities of the ribozyme to destroy endogenous PGRN or sortilin messages and inhibit translation. Since ribozymes unlike antisense molecules, are catalytic, a lower intracellular concentration is required for efficiency.

Aptamers

In another embodiment, the PGRN or sortilin antagonist for use in the methods of the present invention is an aptamer. An aptamer can be a nucleotide or a polypeptide which has a unique sequence, has the property of binding specifically to a desired target (e.g., a polypeptide), and is a specific ligand of a given target. Nucleotide aptamers of the invention include double stranded DNA and single stranded RNA molecules that bind to PGRN or sortilin.

Nucleic acid aptamers are selected using methods known in the art, for example via the Systematic Evolution of Ligands by Exponential Enrichment (SELEX) process. SELEX is a method for the in vitro evolution of nucleic acid molecules with highly specific binding to target molecules as described in, e.g., U.S. Pat. Nos. 5,475,096, 5,580,737, 5,567,588, 5,707,796, 5,763,177, 6,011,577, and 6,699,843, incorporated herein by reference in their entirety. Another screening method to identify aptamers is described in U.S. Pat. No. 5,270,163 (also incorporated herein by reference). The SELEX process is based on the capacity of nucleic acids for forming a variety of two- and three-dimensional structures, as well as the chemical versatility available within the nucleotide monomers to act as ligands (form specific binding pairs) with virtually any chemical compound, whether monomeric or polymeric, including other nucleic acid molecules and polypeptides. Molecules of any size or composition can serve as targets.

The SELEX method involves selection from a mixture of candidate oligonucleotides and step-wise iterations of binding, partitioning and amplification, using the same general selection scheme, to achieve desired binding affinity and selectivity. Starting from a mixture of nucleic acids, preferably comprising a segment of randomized sequence, the SELEX method includes steps of contacting the mixture with the target under conditions favorable for binding; partitioning unbound nucleic acids from those nucleic acids which have bound specifically to target molecules; dissociating the nucleic acid-target complexes; amplifying the nucleic acids dissociated from the nucleic acid-target complexes to yield a ligand enriched mixture of nucleic acids. The steps of binding, partitioning, dissociating and amplifying are repeated through as many cycles as desired to yield highly specific high affinity nucleic acid ligands to the target molecule.

Nucleotide aptamers may be used, for example, as diagnostic tools or as specific inhibitors to dissect intracellular signaling and transport pathways (James, Curr. Opin. Pharmacol. 1:540-546 (2001)). The high affinity and specificity of nucleotide aptamers makes them good candidates for drug discovery. For example, aptamer antagonists to the toxin ricin have been isolated and have IC₅₀ values in the nanomolar range (Hesselberth J R et al., J Biol Chem 275:4937-4942 (2000)). Nucleotide aptamers may also be used against infectious disease, malignancy and viral surface proteins to reduce cellular infectivity.

Nucleotide aptamers for use in the methods of the present invention may be modified (e.g., by modifying the backbone or bases or conjugated to peptides) as described herein for other polynucleotides.

Using the protein structure of PGRN or sortilin, screening for aptamers that act on PGRN or sortilin using the SELEX process would allow for the identification of aptamers that modulate PGRN activity, sortilin activity, or the PGRN-sortilin interaction.

Polypeptide aptamers for use in the methods of the present invention are random peptides selected for their ability to bind to and thereby block the action of PGRN or sortilin. Polypeptide aptamers may include a short variable peptide domain attached at both ends to a protein scaffold. This double structural constraint greatly increases the binding affinity of the peptide aptamer to levels comparable to an antibody's (nanomolar range). See, e.g., Hoppe-Seyler F, et al., J Mol Med 78(8):426-430 (2000). The length of the short variable peptide is typically about 10 to 20 amino acids, and the scaffold may be any protein which has good solubility and compacity properties. One non-limiting example of a scaffold protein is the bacterial protein Thioredoxin-A. See, e.g., Cohen B A et al., Proc. Natl. Acad. Sci. 95(24):14272-14277 (1998).

Polypeptide aptamers are peptides or small polypeptides that act as dominant inhibitors of protein function. Peptide aptamers specifically bind to target proteins, blocking their functional ability (Kolonin et al., Proc. Natl. Acad. Sci. 95:14266-14271 (1998)). Peptide aptamers that bind with high affinity and specificity to a target protein can be isolated by a variety of techniques known in the art. Peptide aptamers can be isolated from random peptide libraries by yeast two-hybrid screens (Xu, C. W., et al., Proc. Natl. Acad. Sci. 94:12473-12478 (1997)) or by ribosome display (Hanes et al., Proc. Natl. Acad. Sci. 94:4937-4942 (1997)). They can also be isolated from phage libraries (Hoogenboom, H. R., et al., Immunotechnology 4:1-20 (1998)) or chemically generated peptide libraries. Additionally, polypeptide aptamers may be selected using the selection of Ligand Regulated Peptide Aptamers (LiRPAs). See, e.g., Binkowski B F, et al., Chem & Biol 12(7): 847-855 (2005), incorporated herein by reference. Although the difficult means by which peptide aptamers are synthesized makes their use more complex than polynucleotide aptamers, they have unlimited chemical diversity. Polynucleotide aptamers are limited because they utilize only the four nucleotide bases, while peptide aptamers would have a much-expanded repertoire (i.e., 20 amino acids).

Peptide aptamers for use in the methods of the present invention may be modified (e.g., conjugated to polymers or fused to proteins) as described for other polypeptides elsewhere herein.

### Compositions

In some embodiments, the methods of the present invention use compositions comprising an antibody or an antigen-binding fragment thereof of the present invention, a polypeptide of the present invention, or a polynucleotide of the present invention.

In some embodiments, the compositions for use in the methods of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol and dextran. Optionally, the suspension may also contain stabilizers. Liposomes can also be used to encapsulate the molecules of this invention for delivery into the cell. Exemplary “pharmaceutically acceptable carriers” are any and all solvents, dispersion media, coatings, antibacterial and antifungal agents, isotonic and absorption delaying agents, and the like that are physiologically compatible, water, saline, phosphate buffered saline, dextrose, glycerol, ethanol and the like, as well as combinations thereof. In some embodiments, the composition comprises isotonic agents, for example, sugars, polyalcohols such as mannitol, sorbitol, or sodium chloride. In some embodiments, the compositions comprise pharmaceutically acceptable substances such as wetting or minor amounts of auxiliary substances such as wetting or emulsifying agents, preservatives or buffers, which enhance the shelf life or effectiveness of the antibodies, antigen-binding fragments, polypeptides, or polynucleotides used in the methods of the invention.

Compositions for use in the methods of the invention may be in a variety of forms, including, for example, liquid, semi-solid and solid dosage forms, such as liquid solutions (e.g., injectable and infusible solutions), dispersions or suspensions. The preferred form depends on the intended mode of administration and therapeutic application. In one embodiment, compositions are in the form of injectable or infusible solutions, such as compositions similar to those used for passive immunization of humans with other antibodies.

The composition can be formulated as a solution, microemulsion, dispersion, liposome, or other ordered structure suitable to high drug concentration. Sterile injectable solutions can be prepared by incorporating an antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In some embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York (1978).

The pharmaceutical compositions of the invention may include a “therapeutically effective amount” or a “prophylactically effective amount” of an antibody, antigen-binding fragment, polypeptide(s), or polynucleotide(s) for use in the methods of the invention. A “therapeutically effective amount” refers to an amount effective, at dosages and for periods of time necessary, to achieve the desired therapeutic result. A therapeutically effective amount may vary according to factors such as the disease state, age, sex, and weight of the individual. A therapeutically effective amount is also one in which any toxic or detrimental effects of the antibody, antigen-binding fragment, polypeptide(s), or polynucleotide(s) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage unit form as used herein refers to physically discrete units suited as unitary dosages for the mammalian subjects to be treated, each unit containing a predetermined quantity of active compound calculated to produce the desired therapeutic effect in association with the required pharmaceutical carrier. The specification for the dosage unit forms of the invention are dictated by and directly dependent on (a) the unique characteristics of the antibody, antigen-binding fragment, polypeptide(s), or polynucleotide(s) and the particular therapeutic or prophylactic effect to be achieved, and (b) the limitations inherent in the art of compounding such an antibody, antigen-binding fragment, polypeptide(s), or polynucleotide(s) for the treatment of sensitivity in individuals.

For treatment with an antibody, antigen-binding fragment, polypeptide(s), or polynucleotide(s) of the invention, the dosage can range, e.g., from about 0.0001 to 100 mg/kg, from about 0.001 to 10 mg/kg, and more usually from about 0.01 to 5 mg/kg, from about 0.01 to 1.0 mg/kg, or from about 0.05 mg/kg to 0.5 mg/kg, of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention (e.g., 0.02 mg/kg, 0.25 mg/kg, 0.5 mg/kg, 0.75 mg/kg, 1 mg/kg, 2 mg/kg, etc.). Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days, or 60 mg/kg weekly.

In the methods of the invention the antibody, antigen-binding fragment, polypeptide(s), or polynucleotide(s) is generally administered directly to the nervous system, intracerebroventricularly, or intrathecally. The invention encompasses any suitable delivery method, however, for delivery to a selected target tissue, including bolus injection of an aqueous solution or implantation of a controlled-release system. Additional suitable delivery methods include oral, parenteral, subcutaneous, intravenous, intramuscular, intraperitoneal, transdermal, intracranial or buccal administration. Use of a controlled-release implant reduces the need for repeat injections. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers 22:547-56 (1985)); poly(2-hydroxyethyl-methacrylate), ethylene vinyl acetate (Langer et al., J. Biomed. Mater. Res. 15:167-277 (1981); Langer, Chem. Tech. 12:98-105 (1982)) or poly-D-(−)-3-hydroxybutyric acid (EP 133,988).

In some embodiments, the antibody, antigen-binding fragment, polypeptide(s), or polynucleotide(s) used in the methods of the invention may be directly infused into the brain. Various implants for direct brain infusion of compounds are known and are effective in the delivery of therapeutic compounds to human patients suffering from neurological disorders. These include chronic infusion into the brain using a pump, stereotactically implanted, temporary interstitial catheters, permanent intracranial catheter implants, and surgically implanted biodegradable implants. See, e.g., Gill et al., supra; Scharfen et al., Int. J. Radiation Oncology Biol. Phys. 24(4):583-91 (1992); Gaspar et al., Int. J. Radiation Oncology Biol. Phys. 43(5):977-82 (1999); Chapter 66, pages 577-580, Bellezza et al., “Stereotactic Interstitial Brachytherapy,” in Gildenberg et al., Textbook of Stereotactic and Functional Neurosurgery, McGraw-Hill (1998); and Brem et al., J. Neuro-Oncology 26:111-23 (1995). Alternative techniques are available and may be applied to administer a compound according to the invention. For example, stereotactic placement of a catheter or implant can be accomplished using the Riechert-Mundinger unit and the ZD (Zamorano-Dujovny) multipurpose localizing unit. A contrast-enhanced computerized tomography (CT) scan, injecting 120 ml of omnipaque, 350 mg iodine/ml, with 2 mm slice thickness can allow three-dimensional multiplanar treatment planning (STP, Fischer, Freiburg, Germany). This equipment permits planning on the basis of magnetic resonance imaging studies, merging the CT and MRI target information for clear target confirmation.

The Leksell stereotactic system (Downs Surgical, Inc., Decatur, Ga.) modified for use with a GE CT scanner (General Electric Company, Milwaukee, Wis.) as well as the Brown-Roberts-Wells (BRW) stereotactic system (Radionics, Burlington, Mass.) can be used for this purpose. Thus, on the morning of the implant, the annular base ring of the BRW stereotactic frame can be attached to the patient's skull. Serial CT sections can be obtained at 3 mm intervals though the (target tissue) region with a graphite rod localizer frame clamped to the base plate. A computerized treatment planning program can be run on a VAX 11/780 computer (Digital Equipment Corporation, Maynard, Mass.) using CT coordinates of the graphite rod images to map between CT space and BRW space.

### Methods

Screening Methods

In some embodiments, the invention provides methods for identifying a compound that modulates the interaction of sortilin and progranulin. The methods of the invention include a method for identifying a compound that modulates the interaction of sortilin and progranulin comprising: (a) mixing a compound with sortilin and progranulin; and (b) measuring the interaction of sortilin and progranulin in the presence of said compound. The method for identifying a compound that modulates the interaction of sortilin and progranulin also comprises comparing the interaction of sortilin and progranulin in the presence of said compound to the interaction of sortilin and progranulin in the absence of said compound.

In some embodiments of the methods of identifying, modulating the interaction between progranulin and sortilin is inhibition of the interaction between progranulin and sortilin. In other embodiments of the methods of the identifying, modulating the interaction between progranulin and sortilin is enhancement of the interaction between progranulin and sortilin. Thus, the methods of screening include identifying those compounds that inhibit or decrease the interaction between progranulin and sortilin and those compounds that enhance or promote the interaction between progranulin and sortilin.

Methods for screening for compounds are known in the art. Such methods include means for identifying or measuring the interaction of sortilin and progranulin, including, but not limited to, assays for measuring the binding of sortilin and progranulin, e.g., the binding and competition assays described in the Examples section below. Additional examples of assays that can be used to identify the binding of sortilin and progranulin include, but are not limited to, immunoblotting and immunoassays, such as ELISAs or RIAs; competitive immunoassays; pull-down assays; immunohistochemistry; surface plasmon resonance using, e.g., a BIACORE™ or KINEXA™ technology; yeast two-hybrid assays; imaging technologies utilizing, e.g., fluorescence resonance energy transfer (FRET); kinetic or thermodynamic methodology using a wide variety of techniques including, but not limited to, microcalorimetry, circular dichroism, capillary zone electrophoresis, nuclear magnetic resonance spectroscopy, fluorescence spectroscopy, and combinations thereof. The screening methods of the invention include in vitro and in vivo methods.

Compounds that can be screened according to the methods of the invention include any of the classes of compounds recited herein. For example, the compounds can be proteins, polypeptides, peptides, antibodies or antigen-binding fragments thereof, polynucleotides, antisense polynucleotides, siRNA, aptamers, or small molecules. The compounds can be screened individually or in parallel. An example of parallel screening is a high throughput drug screen of large libraries of chemicals. Such libraries of compounds can be generated or purchased, e.g., from ChemBridge Corp. Libraries can be designed to cover a diverse range of compounds. For example, a library can include 500, 1000, 10,000, 50,000, or 100,000 or more unique compounds. Alternatively, prior experimentation and anecdotal evidence can suggest a class or category of compounds of enhanced potential. A library can be designed and synthesized to cover such a class of chemicals.

Libraries of compounds may be presented in solution (e.g., Houghten Biotechniques 13:412-421 (1992)), on beads (Lam, Nature 354:82-84 (1991)), chips (Fodor, Nature 364:555-556 (1993)), bacteria (U.S. Pat. No. 5,223,409), spores (U.S. Pat. No. 5,223,409), plasmids (Cull et al., Proc Natl Acad Sci USA 89:1865-1869 (1992)) or on phage (Scott and Smith, Science 249:386-390(1990); Devlin, Science 249:404-406 (1990); Cwirla et al., Proc. Natl. Acad. Sci. 87:6378-6382 (1990); Felici, J. Mol. Biol. 222:301-310 (1991)). Examples of methods for the synthesis of molecular libraries can be found in, for example, DeWitt et al., Proc. Natl. Acad. Sci. U.S.A. 90:6909 (1993); Erb et al., Proc. Natl. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33:2059 (1994); Carell et al., Angew. Chem. Int. Ed. Engl. 33:2061 (1994); and Gallop et al., J. Med. Chem. 37:1233(1994).

Therapeutic or Prophylactic Methods

In some embodiments, the invention provides methods of promoting cell survival by modulating the interaction between progranulin and sortilin. The methods of the invention include a method of promoting cell survival by modulating the interaction between progranulin and sortilin comprising (a) contacting a cell with a compound; and (b) modulating the interaction between progranulin and sortilin. In some embodiments, the compound mimics the binding of progranulin to sortilin.

In some embodiments, the invention provides a method for increasing levels of progranulin in a mammal. The methods of the invention include a method for increasing levels of progranulin in a mammal comprising administering to said mammal a compound that modulates the interaction between progranulin and sortilin, a compound that modulates the activity of sortilin, a compound that modulates the activity of progranulin, or a compound that modulates the activity of sortilin and progranulin.

In some embodiments, the invention provides methods for modulating the interaction between progranulin and sortilin, the activity of progranulin or sortilin, or the activity of progranulin and sortilin. The methods of the invention include a method for modulating the interaction between progranulin and sortilin in a mammal comprising administering to said mammal a therapeutically effective amount of a compound capable of modulating said interaction. The methods of the invention include a method for modulating the activity of sortilin in a mammal comprising administering to said mammal a therapeutically effective amount of a compound capable of modulating the interaction between sortilin and a progranulin. The methods of the invention also include a method for modulating the activity of progranulin in a mammal comprising administering to said mammal a therapeutically effective amount of a compound capable of modulating the interaction between sortilin and progranulin.

In some embodiments, the methods include modulating the interaction between sortilin and progranulin to reduce the signs or symptoms of a disease, disorder, or condition in a mammal in need thereof.

In some embodiments, the invention provides methods of treating or preventing a disease, disorder, or condition by modulating the interaction between progranulin and sortilin, the activity of progranulin or sortilin, or the activity of progranulin and sortilin. The methods of the invention include a method of treating or preventing a disease, disorder, or condition comprising administering to a mammal in need thereof a therapeutically or prophylactically effective amount of a compound capable of modulating the interaction between sortilin and a progranulin. The methods of the invention include a method of treating or preventing a disease, disorder, or condition comprising administering to a mammal in need thereof a therapeutically or prophylactically effective amount of a compound capable of modulating the activity of sortilin. The methods of the invention include a method of treating or preventing a disease, disorder, or condition comprising administering to a mammal in need thereof a therapeutically or prophylactically effective amount of a compound capable of modulating the activity of progranulin. The methods of the invention also include a method of treating or preventing a disease, disorder, or condition comprising administering to a mammal in need thereof a therapeutically or prophylactically effective amount of a compound identified in the screening methods provided herein.

In some embodiments of the invention, the disease, disorder, or condition is selected from the group consisting of frontotemporal dementia (FTD) and amyotrophic lateral sclerosis (ALS). In one embodiment, the disease, disorder, or condition is FTD. In some embodiments, the mammal is a human.

In some embodiments of the methods of the invention, modulating the interaction between progranulin and sortilin, the activity of sortilin, or the activity of progranulin is inhibition of the interaction between progranulin and sortilin, the activity of sortilin, or the activity of progranulin. In other embodiments of the methods of the invention, modulating the interaction between progranulin and sortilin, the activity of sortilin, or the activity of progranulin is enhancement of the interaction between progranulin and sortilin, the activity of sortilin, or the activity of progranulin.

In some embodiments of the invention, the compound administered in the methods of the invention include any of the compounds identified in the screening methods of the invention and any of the compounds recited herein, including combinations of the compounds. In one embodiment, the compound is selected from the group consisting of an antibody, or antigen-binding fragment thereof; a polypeptide; a polynucleotide; and combinations thereof. The antibody, or antigen-binding fragment thereof, used in the methods of the invention include an antibody, or antigen-binding fragment thereof that specifically binds a sortilin polypeptide. Sortilin polypeptides for use in the methods of the invention comprise an amino acid sequence selected from the group consisting of SEQ ID NO:21, SEQ ID NO:34, and SEQ ID NO:36. The antibody or antigen-binding fragment thereof includes those selected from the group consisting of a polyclonal antibody, a monoclonal antibody, a Fab fragment, a Fab′ fragment, a F(ab′)₂ fragment, an Fv fragment, an Fd fragment, a diabody, and a single-chain antibody.

In particular embodiments, the polypeptide used in the methods of the invention comprises a polypeptide selected from the group consisting of a sortilin polypeptide; a fragment of a sortilin polypeptide; a polypeptide selected from the group consisting of SEQ ID NO:21, SEQ ID NO:34, and SEQ ID NO:36; a progranulin polypeptide; a fragment of a progranulin polypeptide; a polypeptide selected from the group consisting of SEQ ID NO:30 and SEQ ID NO:32; a progranulin polypeptide comprising the C-terminal 17 amino acids of any one of SEQ ID NO:30 and SEQ ID NO:32; and a variant, derivative, or analog thereof. In one embodiment the polypeptide is a fusion protein comprising a heterologous polypeptide or a polymer. In the methods of the invention, the heterologous polypeptide of the fusion protein is selected from the group consisting of serum albumin; an Fc region selected from the group consisting of an IgA Fc region, an IgD Fc region, an IgG Fc region, an IgE Fc region, and an IgM Fc region; a signal peptide; a polypeptide tag selected from the group consisting of a FLAG tag; a Strep tag; a poly-histidine tag; a VSV-G tag; an influenza virus hemagglutinin (HA) tag; and a c-Myc tag; and a combination of one or more thereof. In a particular embodiment, the polymer of the fusion protein is selected from the group consisting of a polyalkylene glycol; a sugar polymer; a polypeptide; and a combination one or more thereof. The polypeptides for use in the methods of the invention can be conjugated to 1, 2, 3, or 4 polymers and have a total molecular weight of polymers from 5,000 Da to 100,000 Da.

In one embodiment, the polypeptide used in the methods of the invention is a cyclic polypeptide. The cyclic polypeptide further comprises a first molecule linked at the N-terminus and a second molecule linked at the C-terminus and the first molecule and the second molecule can be joined to each other to form the cyclic molecule. In a particular embodiment, the first and second molecules are selected from the group consisting of: a biotin molecule, a cysteine residue, and an acetylated cysteine residue.

In particular embodiments, the polynucleotides used in the methods of the invention comprise an isolated polynucleotide selected from the group consisting of an antisense polynucleotide; a ribozyme; a small interfering RNA (siRNA); a small-hairpin RNA (shRNA); and a combination of one or more thereof. In some embodiments, the polynucleotide comprises a sortilin nucleic acid or a progranulin nucleic acid. In one embodiment, the polynucleotide is an antisense polynucleotide comprising at least 10 bases complementary to the coding region of sortilin mRNA or progranulin mRNA.

### EXAMPLES

### Example 1

### Identification of a High-Affinity Receptor for Progranulin (PGRN) in Neurons

In order to define PGRN's mechanism of action and elucidate PGRN's contribution to FTD and ALS, the high-affinity receptor through which PGRN signals in the brain was identified.

1.1 Creation of Tagged PGRN Proteins

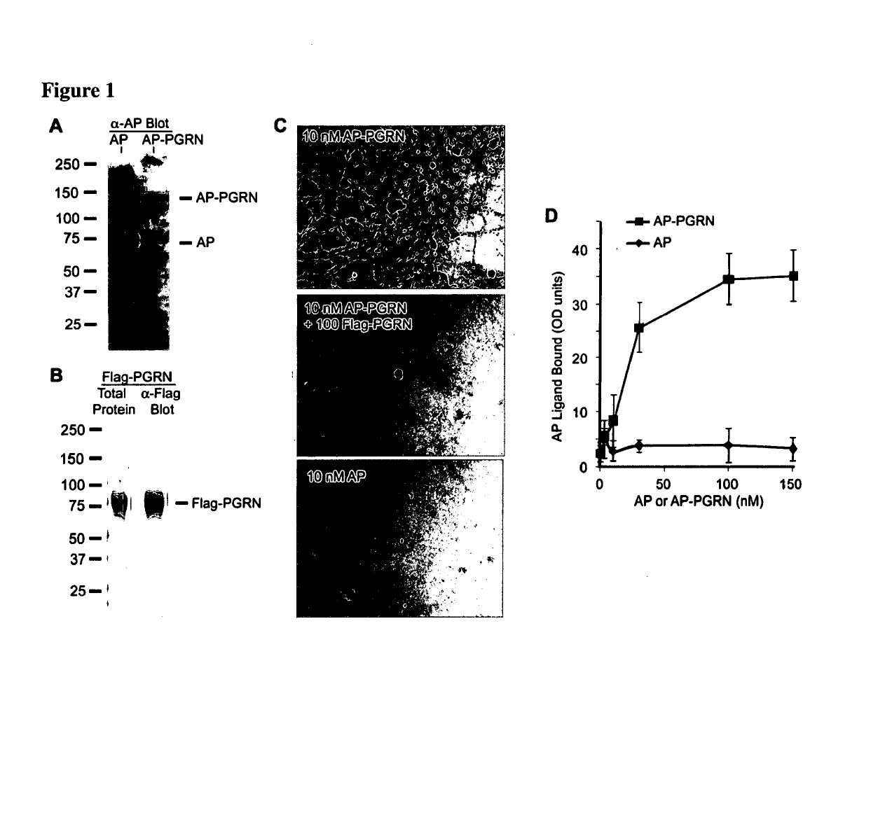

Tagged versions of the PGRN protein (huPGRN, GenBank Accession No. BC010577) were produced and purified. First, either alkaline-phosphatase (AP) or AP-PGRN protein were produced in HEK293T cells (ATCC No. CRL-1573) by transfection. Transfection was performed using FUGENE® 6 according to manufacturer's specifications (Roche) with the expression vector pAPtag-5 (Flanagan, J. G., et al., Methods Enzymol. 327:19-35 (2000)). FIG. 1A shows that the transfected cells produced an AP immunoreactive protein of the expected size: for PGRN ˜75 kDa and for AP ˜70 kDa (on a denaturing SDS-PAGE gel). Second, FLAG-tagged PGRN (Flag-PGRN) was also produced by HEK293T cell transfection with appropriate vectors (pSectag2A, Invitrogen). The resulting fusion protein was then purified to homogeneity by affinity chromatography using an anti-FLAG affinity gel (Sigma), as demonstrated by Coomassie Blue staining of total protein (“Total Protein” lane) and by anti-FLAG immunoblot (“α-Flag Blot” lane) in FIG. 1B. Aliquots of the fusion protein were run on an SDS-PAGE 4-20% gradient denaturing gel, and immunoblotting was carried out by transferring the gel proteins to an Immobilon-FL PVDF membrane and visualized using a LI-COR® ODYSSEY® machine at 680 nM.

1.2 Binding Assays Using Rat and Mouse Cortical Neurons

Rat (Sprague-Dawley) cortical neurons were isolated from E18 embryos and cultured in vitro for 3-21 days. Cultures were maintained in Neurobasal-A medium supplemented with B27, GLUTAMAX™ (2 mM), sodium pyruvate (1 mM), penicillin, and streptomycin. The binding of 10 nM AP-PGRN or 10 nM AP was detected as a dark reaction product produced by alkaline phosphatase (AP). Binding was measured as follows. The AP tagged ligand (in NBA) was incubated with the cortical neurons for 1 hr at 4° C., washed three times with HBH buffer, fixed with formaldehyde, and after washing again with HBH the neurons were incubated in a 67° C. oven to inactivate the endogenous AP. The tagged AP was then visualized using AP substrate 5-bromo-4-chloro-3-indolyl phosphate/nitro blue tetrazolium, which results in a dark precipitate. FIG. 1C shows that 10 nM AP-PRGN specifically and discreetly bound to the cultured cortical neurons (FIG. 1C, top panel), whereas 10 nM AP alone did not (FIG. 1C, bottom panel). The addition of 100 nM Flag-PGRN displaced 10 nM AP-PGRN (FIG. 1C, middle panel).

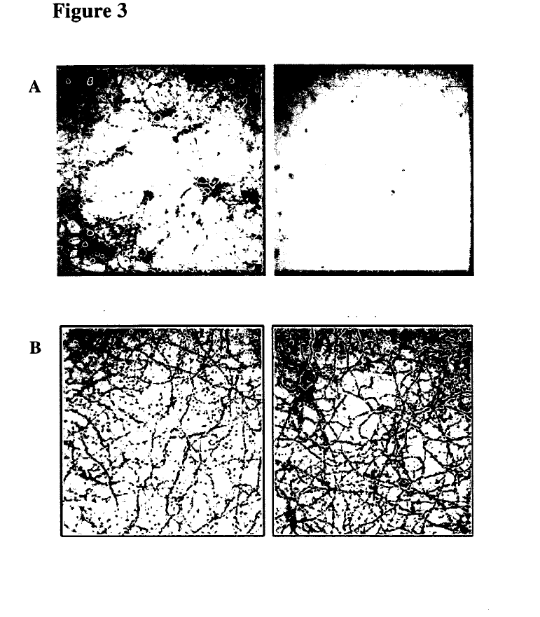

Similarly, as shown in FIG. 3A, the binding of 5 nM AP-PGRN (left panel) is displaced by the addition of 100 nM unlabeled PGRN (right panel) in mouse E17 cortical neurons prepared under the same conditions as described above. Binding was measured according to the methods described above by preincubating 100 nM PGRN with the cortical neurons for 1 hr and using a 5 nM AP-PGRN and 100 nM untagged PGRN mixture. Thus, high affinity neuronal PGRN binding sites are saturable (FIG. 1D), can be displaceable by excess (100 nM) tagged or untagged PGRN (FIGS. 1C and 3A), and exhibit ˜20 nM affinity for PGRN. Binding sites were concentrated in dendrites, and rare in axons. By contrast, binding assays using GRN-E-AP fusion protein shows an indistinguishable detectable affinity for cortical neurons (data not shown).

1.3 Identification of Sortilin-1 (Sort1) as the PGRN Neuronal Binding Site

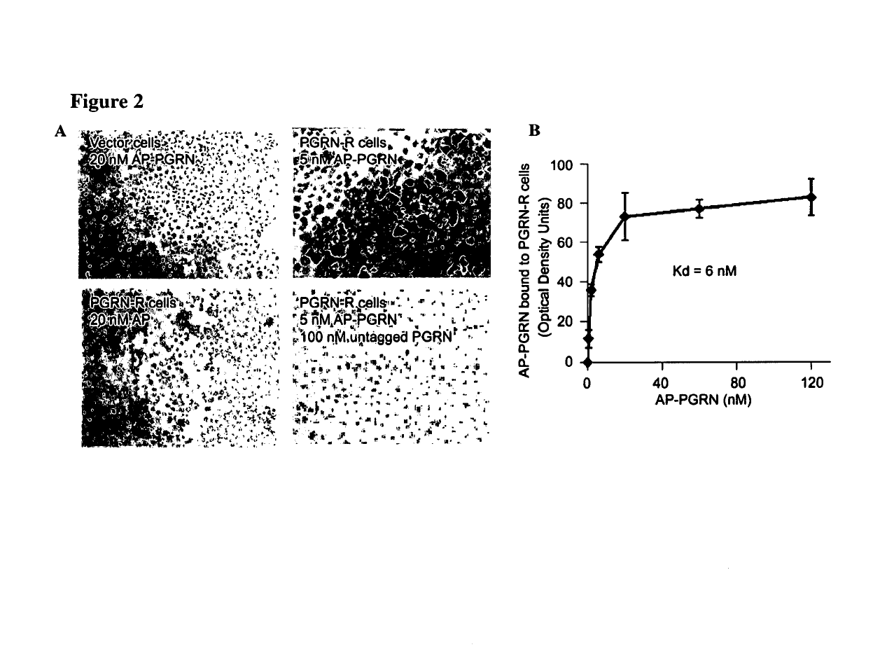

To determine the molecular nature of the neuronal binding site of PGRN, expression cloning experiments utilizing AP-PGRN as a tagged ligand were conducted. COS-7 (ATCC No. CRL-1651) cells were transfected with expression vectors containing mouse cDNAs using FUGENE® 6 according to manufacturer's specifications (Roche). An arrayed adult mouse brain cDNA library (OriGene) of 225,000 clones was screened in pools of 5,000 at a time, as well as a library consisting of 352 individual preparations of cDNAs encoding transmembrane proteins (GFC-transfection array panel, OriGene). The COS-7 cells that specifically bound AP-PGRN (5 nM) contained a single cDNA clone, which encoded a single-pass, membrane-spanning surface glycoprotein that supports high affinity AP-PGRN binding (FIG. 2A, upper-right panel). DNA sequencing demonstrated that the clone encoded sortilin-1 (Sort1; GenBank Accession No. NM 019972), which we have identified as the PGRN receptor (PGRN-R). 20 nM AP-PGRN did not bind to COS-7 cells transfected with empty vector (FIG. 2A, upper-left panel), and 20 nM AP alone did not bind to COS-7 cells transfected with the Sort-1 cDNA clone (FIG. 2A, lower-left panel). As in mouse cortical neurons, binding of 5 nM AP-PGRN to Sort-1 (PGRN-R) in COS-7 cells was displaceable by excess untagged PGRN (100 nM) and was saturable, with an apparent Kd of 6 nM (FIG. 2A, lower-right panel, and 2B). The binding affinity of AP-PRGN measured for Sort1-transfected COS-7 cells and for neurons was indistinguishable, with a Kd of 5-10 nM (FIG. 2B, 6 nM, and data not shown). In both cases, unlabeled 100 nM PGRN displaced binding (FIG. 2A, lower-right panel, and FIG. 3A, right panel).

Purified PGRN protein (Flag tagged) and the purified ectodomain of Sort1 (amino acids 60-725 of the human sequence) formed a physical complex that was isolated by affinity chromatography (data not shown). In addition, PRGN-E affinity chromatography pulled down Sort1 from brain extracts (data not shown). For affinity chromatography, glutathione S-transferase (GST) fused to the C-terminal segment of PGRN (amino acids 495 to the end) was produced in E. coli as a 45 kDa protein and then purified on glutathione-sepharose resin. Brain lysate was prepared from mouse brain by homogenization in RIPA buffer and then incubated with the GST-PGRN containing resin. After washing, bound protein was eluted with SDS and analyzed by anti-sortilin immunoblot. An immunoreactive Sort1 band was detected with lysates from wild type brain but not from Sort1−/− brain, and with GST-PGRN but not with GST control resin. Thus, it can be unexpectedly concluded from these studies that PGRN and sortilin interact in a ligand/receptor-type complex on the cell surface.

1.4 PRGN Binding is Highly Specific for Sort1

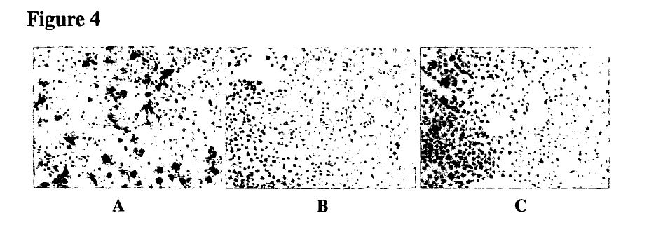

PGRN binds to sortilin of mouse or human origin (data not shown), but PGRN does not bind to two Vps10p-related receptor proteins, SorLA and SorCS1. The human SorLA expression vector was provided by Anders Nykjaer, M.D., Ph.D., Aarhus University, Aarhus, Denmark (Jacobsen, L., et al., J. Biol. Chem. 276:22788-22796 (2001); Gliemann, J., et al., Biochem J. 381:203-212 (2004); GenBank Accession No. U60975), and the mouse SorCS1 expression vector in pCMV-Sport6 was obtained from Open Biosystems (GenBank Accession No. BC007486). When SorLA and SorCS1 expression vectors were expressed in COS-7 cells, 120 nM AP-PGRN exhibited no affinity for SorLA or SorCS1 (FIGS. 4B and 4C), while 20 nM AP-PGRN bound COS-7 cells transfected with sortilin (FIG. 4A). A fragment of PGRN, termed PGRN-E in the literature, binds to sortilin with the same affinity as does full length PGRN (data not shown).

A region of PGRN that interacts with sortilin has been mapped using shorter fragments of the full-length PGRN fused to AP, which were then used to conduct binding assays with sortilin transfected COS-7 cells. The binding interaction was localized to the carboxyl terminal 17 amino acids of PRGN (amino acids 577-593 of GenBank Accession No. NP 002078) using Sort1-expressing COS-7 binding experiments (data not shown). This 17-amino acid region of PRGN also mediates the high affinity binding to neurons (data not shown).

1.5 Sortilin Knock Out Mice have More Unbound PGRN

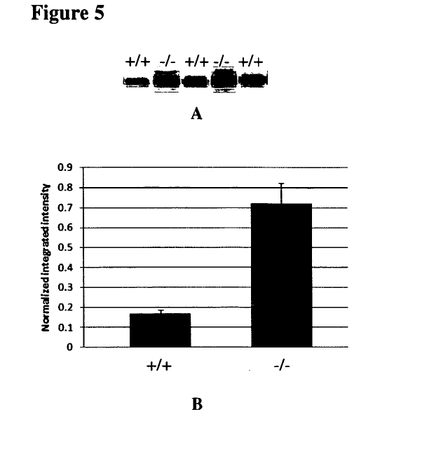

Gene-targeted sortilin −/− and +/− mice from Anders Nykjaer, M.D., Ph.D., Aarhus University, Aarhus, Denmark, were used to obtain cultures of cortical neurons. The binding of 2 nM AP-PGRN to cortical neurons isolated from sortilin −/− (FIG. 3B, left panel) and +/− (FIG. 3B, right panel) mice was evaluated. AP-PRGN binding was reduced to 30% of control values, indicating that sortilin was the predominant cell surface binding site for PRGN in neurons. Incubation of mouse cortical neurons and binding assays were conducted as described above. Additional studies of the sortilin −/− mice were conducted. Serum isolated from WT (+/+) mice and sortilin −/− mice showed increased levels of PGRN in the sortilin −/− mice (FIGS. 5A and 5B). In addition to serum, brain levels of PGRN were markedly elevated in the sortilin −/− mice (data not shown). The fold-increase in PGRN was approximately 4-fold in serum and 3-fold in brain, verifying that the two proteins interact in vivo (data not shown). PGRN levels were obtained by collecting blood samples, separating the serum by centrifugation at 20,000×g for 20 min, and removing the serum albumin/IgG using ProteoExtract (CALBIOCHEM®). Thereafter, equal amounts of serum was separated on a denaturing SDS-PAGE gel and probed for PGRN by immunoblot.

### Example 2

### Examination of PGRN Effects on Cerebral Cortical Cultures

2.1 Cortical Neuron Survival

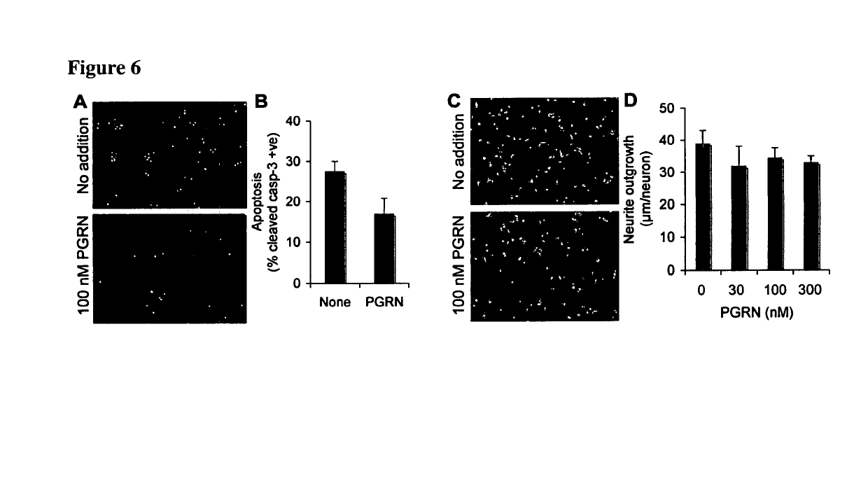

In order to examine the PGRN-Sort1 interaction and the effects of PGRN on cerebral cortical cultures, apoptosis assays were performed. Cultures of cortical neurons were established from E18 rat embryo cerebral cortex, as described above, and maintained for 7 days in vitro. Then, the cells were exposed to vehicle or 100 nM purified PGRN protein for 36 hours. The cultures were maintained as mentioned above, and Flag-PGRN diluted in PBS or PBS vehicle were incubated in the cultures for 36 h. Thereafter, the cells were fixed with FA 15 mts and stained with anti-cleaved caspase 3 antibody (Cell Signaling) to detect apoptotic cells (FIG. 6A; shown in white) and with DAPI to detect all nuclei (FIG. 6A; shown in gray). The number of cells positive for cleaved caspase-3 were less in the PGRN-treated culture (FIGS. 6A and B), indicating that PRGN-Sort1 signaling plays a role in modulating apoptosis and thus, cell survival. These finding may relate indirectly to one study of immortalized cells linking PGRN to caspase-mediated cleavage of TDP43 (Zhang, Y. J., et al., J. Neurosci. 27:10530-10534 (2007)).

2.2 Outgrowth Assay

To further examine the PGRN-Sort1 interaction, neurite outgrowth assays were performed. Cortical neurons were isolated from E18 rat embryos and cultured for 1 day in vitro with or without 100 nM purified Flag-PGRN protein, as described above. Neurites were then visualized with an anti-Beta III tubulin (Promega) using an automated image acquisition. Neurite outgrowth analysis was done using ImageXpress (Molecular Devices), and average neurite length was calculated. No PGRN-induced change in cortical neurite outgrowth was detected under the same conditions that produce PGRN regulation of apoptosis (FIGS. 6C and D). Although this finding differs from a recent report (Van Damme, P., et al., J. Cell Biol. 181:37-41 (2008)), it is possible that cell survival is the primary action of PGRN, and any outgrowth effect is secondary.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

1. A method for identifying a compound that modulates the interaction of sortilin and progranulin comprising:
(a) mixing a compound with sortilin and progranulin; and
(b) measuring the interaction of sortilin and progranulin in the presence of said compound.

2. The method of claim 1 further comprising comparing the interaction of sortilin and progranulin in the presence of said compound to the interaction of sortilin and progranulin in the absence of said compound.

3. A method of promoting cell survival by modulating the interaction between progranulin and sortilin comprising:
(a) contacting a cell with a compound; and
(b) modulating the interaction between progranulin and sortilin.

4. The method of claim 3 wherein said compound promotes or mimics the binding of progranulin to sortilin.

5. A method for increasing levels of progranulin in a mammal comprising administering to said mammal a compound that modulates the interaction between progranulin and sortilin.

6. (canceled)

7. A method for modulating the interaction between progranulin and sortilin in a mammal comprising administering to said mammal a therapeutically effective amount of a compound capable of modulating said interaction.

8. (canceled)

9. (canceled)

10. A method of treating or preventing a disease, disorder, or condition comprising administering to a mammal in need thereof a therapeutically or prophylactically effective amount of a compound, wherein said compound is selected from the group consisting of:
(a) a compound capable of modulating the interaction between sortilin and a progranulin;
(b) a compound capable of modulating the activity of sortilin; and
(c) a compound identified according to the method of claim 1.

11. The method of claim 10, wherein said disease, disorder, or condition is selected from the group consisting of frontotemporal dementia (FTD) and amyotrophic lateral sclerosis (ALS).

12. The method of claim 10, wherein said disease, disorder, or condition is frontotemporal dementia (FTD).

13. (canceled)

14. (canceled)

15. (canceled)

16. The method of claim 3, wherein said compound is selected from the group consisting of:
(a) an antibody, or antigen-binding fragment thereof;
(b) a polypeptide;
(c) a polynucleotide; and
(d) a combination of one or more of (a)-(c).

17. The method of claim 16, wherein said antibody, or antigen-binding fragment thereof, specifically binds a sortilin polypeptide.

18. The method of claim 17, wherein said sortilin polypeptide comprises an amino acid sequence selected from the group consisting of SEQ ID NO:21, SEQ ID NO:34, and SEQ ID NO:36.

19. (canceled)

20. The method of claim 16, wherein said polypeptide comprises a polypeptide selected from the group consisting of:
(a) a sortilin polypeptide;
(b) a fragment of a sortilin polypeptide;
(c) a polypeptide selected from the group consisting of SEQ ID NO:21, SEQ ID NO:34, and SEQ ID NO:36;
(d) a progranulin polypeptide;
(e) a fragment of a progranulin polypeptide;
(f) a polypeptide selected from the group consisting of SEQ ID NO:30 and SEQ ID NO:32;
(g) a progranulin polypeptide comprising amino acids 577-593 of SEQ ID NO:30 or amino acids 584-602 of SEQ ID NO:32;
(h) a polypeptide that is 70%, 75%, 80%, 85%, 90%, 95%, or 100% identical to a reference amino acid sequence selected from the group consisting of: (i) SEQ ID NO: 10, (ii) SEQ ID NO: 11, (iii) SEQ ID NO:12, (iv) SEQ ID NO:13, (v) SEQ ID NO:14, (vi) SEQ ID NO: SEQ ID NO:15, (vii) SEQ ID NO:16, (viii) SEQ ID NO:17, (ix) SEQ ID NO:18, (x) SEQ ID NO:19, (xi) SEQ ID NO:21, (xii) SEQ ID NO:30, (xiii) SEQ ID NO:32, (xiv) SEQ ID NO:34, and (xv) SEQ ID NO:36, and
(i) a variant, derivative, or analog of any one of (a)-(h).

21. The method of claim 20, wherein said polypeptide is a fusion protein comprising a heterologous polypeptide or a polymer.

22. The method of claim 21, wherein said heterologous polypeptide is selected from the group consisting of:
(a) serum albumin;
(b) an Fc region selected from the group consisting of an IgA Fc region, an IgD Fc region, an IgG Fc region, an IgE Fc region, and an IgM Fc region;
(c) a signal peptide;
(d) a polypeptide tag selected from the group consisting of a FLAG tag; a Strep tag; a poly-histidine tag; a VSV-G tag; an influenza virus hemagglutinin (HA) tag; and a c-Myc tag; and
(e) a combination of one or more of (a)-(d).

23. The method of claim 21, wherein said polymer is selected from the group consisting of:
(a) a polyalkylene glycol;
(b) a sugar polymer;
(c) a polypeptide; and
(d) a combination one or more of (a)-(c).

24. (canceled)

25. (canceled)

26. The method of claim 16, wherein said polypeptide is a cyclic polypeptide.

27. (canceled)

28. (canceled)

29. The method of claim 16, wherein said polynucleotide comprises an isolated polynucleotide selected from the group consisting of:
(a) an antisense polynucleotide;
(b) a ribozyme;
(c) a small interfering RNA (siRNA);
(d) a small-hairpin RNA (shRNA); and
(e) a combination of one or more of (a)-(d).

30. The method of claim 29, wherein said polynucleotide comprises a sortilin nucleic acid or a progranulin nucleic acid.

31. (canceled)

32. The method of claim 7, wherein modulating said interaction between sortilin and progranulin reduces signs or symptoms of a disease, disorder, or condition in a mammal in need thereof.
